SEQUENCE LISTING (84 sequences)

SEQ ID NO: 1 - DNA, 3888 bp, Solanum lycopersicum
  source 1..3888: mol_type = genomic DNA
aagtcaagaaacaggttactcctgtttgagtgaggaaaagttggtttgcctgtctgtggtctttttataatctttttctacagaagagaaagtgggtaattttgtttgagagtggaaatattctctagtgggaatctactaggagtaatttattttctataaactaagtaaagtttggaaggtgacaaaaagaaagacaaaaatcttggaattgttttagacaaccaaggttttcttgctcagaatgtctgttgccttgttatgggttgtttctccttgtgacgtctcaaatgggacaagtttcatggaatcagtccgggagggaaaccgtttttttgattcatcgaggcataggaatttggtgtccaatgagagaatcaatagaggtggtggaaagcaaactaataatggacggaaattttctgtacggtctgctattttggctactccatctggagaacggacgatgacatcggaacagatggtctatgatgtggttttgaggcaggcagccttggtgaagaggcaactgagatctaccaatgagttagaagtgaagccggatatacctattccggggaatttgggcttgttgagtgaagcatatgataggtgtggtgaagtatgtgcagagtatgcaaagacgtttaacttaggttagcttcttcaatctattcattcgtttaccaaatattatttggtaagcactaattatgaatatatatatgttcatgttattgatgaagacaaaatttgatctttgtttgtttattcaggaactatgctaatgactcccgagagaagaagggctatctgggcaatatatggtgaggtttctagccatttaataacagttacgcgcacaaacacatatgattaatcggggacgagaaaaaaagaaatgaagtttgagttttgagggtcatatgtaataggtaaatccgagcttgactagcttgagatgtttattgtcatatcatgctcaatactaaccaaaacactgaaaaagaacttgattatatttacatactaatattttcatttgcgttgctgttcacatttttacctatggaactggtttttgtgatttgttatacttcatattcgatgttaataaaatatatcattcctccctttttctccacttcaagctttactgtagtgttgaaaggggaaactccttttaatgattgcatatataaacgaacttcttgagttgaatagtttctcattatgatctgtttaaacagtatggtgcagaagaacagatgaacttgttgatggcccaaacgcatcatatattaccccggcagccttagataggtgggaaaataggctagaagatgttttcaatgggcggccatttgacatgctcgatggtgctttgtccgatacagtttctaactttccagttgatattcaggttagtctaccaattctatggtctttatatttgttcaatttgcgtttgatgtcacttttgctgagggcttttctaatagcttacttcagcctagcggaaatgtttgtagttgaatctctagttctgtctcctatatctgtttctctcgtcctagatactacacatacttcatttctgttttaacattttattcgtcttttggtgttgttttgtatgtgaatcatatatttggaacagaatcattattagttcacatgatttcatttgctttcttcaatagcgtaattgtctaaccttccaatatatgttgcagccattcagagatatgattgaaggaatgcgtatggacttgagaaaatcgagatacaaaaacttcgacgaactatacctttattgttattatgttgctggtacggttgggttgatgagtgttccaattatgggtatcgcccctgaatcaaaggcaacaacagagagcgtatataatgctgctttggctctggggatcgcaaatcaattaactaacatactcagagatgttggagaagagtaagtacaaagctgtgttttacgcacataattttttttgctaatatttacatatcaaaatataggaaaatgagctcttcggttatccggtttatattttttttatgtcaacataatagtataaagtaattagtatcagtcgttctgggaataaaattgcagaactcaatttagccgtgttgtgaaatcctgcttgttttgagagcttaaagctcattagttagtcgttagagacgaagaaattcttcgttgtccatctttattccaccttaaagttgtgatattttcattattggtacatttggcaaaaacacctgaacaaatttatgacggatgccttttgaaagtcactatacctgtctagtcggcgtttatcacatttctttgacatattgaactttgaaacatgatatcagctctagacagtgacgagccatgatcaatttctttcctttattctttctttggaagtgccgtatttaggcttccgttgttcttatatattgctttccctgcagtgccagaagaggaagagtctacttgcctcaagatgaattagcacaggcaggtctatccgatgaagatatatttgctggaagggtgaccgataaatggagaatctttatgaagaaacaaatacatagggcaagaaagttctttgatgaggcagagaaaggcgtgacagaattgagctcagctagtagattccctgtaagcattcgtaaactctttagttttatgaaatgattcttttttcgcgttattagatgaatatggttgcttgtgttgagtatttctaggtcgatgaagttgagacaagggtttttaagttttaacgacttttacggggtgccatgttatctgctacctaatcttaggtagttgaccggaagggctagaattttaacctcatgttcaccctaccaaccaagaaatgaacctcgcatagagctcgtagttatgaatatttgctttggcatgacattgtgcggatcatgaaatgtcttagattatatggaaaaatcattctattacatcgaatagatacattagatctaagaagcacgccgtgttgtaaatgagaaattctatagctcagatctttagttttctctgaacgacctacaaaccaacggataaccttgtattgagcttgtcgttctcagtatttgcactaacattacgtcgtgtggatcctgaaatggcttggattgctattattctggatatggcaaaaccattttattagtactagatatcgaataactacatttgaccctacaagtaccctgggttggagttacaatatcccatacctcgtatctttagtgttctcttatttatcacctttgtctactattctggcaaaataacctcactcgttactcggtgttttccaggtatgggcatctttggtcttgtaccgcaaaatactagatgagattgaagccaatgactacaacaacttcacaaagagagcatatgtgagcaaatcaaagaagttgattgcattacctattgcatatgcaaaatctcttgtgcctcctacaaaaactgcctctcttcaaagataaagcatgaaatgaagatatatatatatatatatatagcaatatacattagaagaaaaaaaggaagaagaaatgttgttgtattgatataaatgtatatcataaatattaggttgtagtaacattcaatataattatctcttgtagttgttgtatcttcactttatctcaactcctttgagagaactttccgtagttatctgctttgcacttggttactcagaattttactgtgggcatgataattgatataccaaattcagttttgattctatcgaaaaatttgttattacatttttttggggggaaaggaattatctcagatatgacgattttacatttcaa

SEQ ID NO: 2 - DNA, 2467 bp, Solanum lycopersicum
  source 1..2467: mol_type = genomic DNA
ggtgccaacacgttgtttgcactctatttatacactttccacaaatattccacaaactaaaaatccatcgtattcgtatatttaaccttccctttcattttcactgaatttggatcctccaccgtccatggcggccggaatttcagcctccgctagttcccgaaccattcgcctccgtcataacccgtttctcagtccaaaatccgcctcaaccgccccgccggttctgttcttctctccgttaactcgcaattttggcgcaattttgctgtctagaagaaagccgagattggcggtttgttttgtgctggagaatgagaaattgaatagtactatcgaaagtgagagtgaagtaatagaggatcggatacaagtagagattaatgaggagaagagtttagctgccagttggctggcggagaaattggcgaggaagaaatcggagaggtttacttatcttgtggcagctgtgatgtctagtttggggattacttctatggcgattttggcggtttattacagattttcatggcaaatggaggtactaattaatgaatcactccattcttctttcttctttcttaagcttaatcttaaagctttttgatagggtggagaagtgcctttttctgaaatgttagctacattcactctctcgtttggcgctgccgtaagttcctttttttttcttctttcacatgttcgattcttcttcccattttgtctaatttgtgtgatataatttaaattttgagaattaaatattttgatttttgattttttttcaactcaaaattcaaactatgtgatataaattacatataatacataaatatgtactttaacttggcttcaaatcacatttatgtcattcaattttggatgtgttcaagtagatacttaaatttgtataaaattgaacaaatggacacacatgttctacatgtgatcctacatgtcattttttgtcttacattatgtcttatgtgtattatgtcatgtaggattcatgtgtttatttttttaaatgttagataatcaaagtgtttgtttgtgcattatgaaagttggaggtcaaatttaaaattcaatatatctataataaaatgtttaaaaagtaatagaagtttcaaaaatgagttgatttgctgtaatttaatgctgtggtccttcaggtaggaatggagtactgggcgagatgggctcatagagcactatggcatgcttctttatggcacatgcacgaggttaatttccaaatctttacactaaattattaggcactaatttggattcttatttaaaaaaaaataaagattcttgtcaaagtttttcaaaattttgactgttcatgatttttatataaatatttgcagtcgcaccatagaccaagagaaggaccttttgagatgaacgacgttttcgccataacaaatgctgttccagctatagctcttctttcctacggtttcttccataaagggatcgtccctggcctctgtttcggcgctgtatgtaatcatcgactaattatatgtatcgattttttttttatgagtttaagttatatatattaatgcggtttttatctccagggattggggatcacagtatttgggatggcttacatgttcgttcacgatggactggttcataagagatttcccgtagggcctattgccaacgtgccttactttcggagggtagctgcagcacatcaggtaataaaaagtttattggactgtagctatactcttaatcttactttaatcaaaattagtaccatcatttaaatagattaatatttaattgattgtcttttttttaattaatttgtattttaacgactcaatcttctttgattttgctgtctggggatttcagcttcatcactcggacaaatttgatggtgtcccatatggcttgttcctaggacctaaggtgctactttttccctcccttttttcgtcgaatgaattacgatatactgttaattcgaaaattttagtagctcagttcattgattttttgaattgttactttatcaatgtgggtttgattcccttaccccaaattttatttttaatatggattaatggctatttggtgcaggaattggaagaagtaggaggacttgaagagttagaaaaggaagtcaaccgaaggattaaaatttctaagggattattatgatcaaaagatacgtctgataataataaaatgcgattgtatttaggctgtagattattattgggaaaaagatagaaagatatatatatgaatataatataaaatgcaacaagctttctatggagaagtccttttcttttttggttcctgtacgtaaaaggtgaacaatttgatgtcctagtacttgttgacaaaccagaagaacgataattcaaaacaaagaaatgtacaattacacttatcgtattggataataagaatatagtgttttggtcgt

SEQ ID NO: 3 - DNA, 11649 bp, Solanum lycopersicum
  source 1..11649: mol_type = genomic DNA
ttccattataaatttcttctttttcagtctaccaaacttggtgagttttctctatctctctatgcgtgttaattttttaattaaaaaaataataatacttttgtgatcacttttatcttttctttgcatgaaattttgttgaaatctttagagcatctatcacgtcatcctattgataaaaaaagtgagaaatatgaaaaataatttcaatctacatgtaggataaaatttaataattttaactcaaaatttgtatttgtattaaaaagaatctgttactttttctattttaattgttaaaatttaatcataatctttttttcatataccttaaatattcaatcatgtaatttataatatcgtttacgtagtttttaaatatataagtttaattttaaaaaaaacataaaaattttatattttaaatattgcttaaatttaaattgtttgactcttaaaatttaatatatatatatatatatatatatatatattaagatagaagagtaatatttgtgaataatttattaagaacacaataagtagaagaaaagaaagtgatgtgaaatttataaactaaaaagggagaatccatcggcgatcatctaaatttgacataattttttacttagatattttaactaaattttgtttattttagacattgcgtgtcatgtataactgtgtcattttgatactttttcgataatctgccaaatttttagatgtatataataacgtgtcaaaatgacgaattaaataaagacatgtgacatatataaaaaaaattaattattaaatgatgaattttgagtatgaaaaaaactaacaaatgactcattgacatgtggaatttcattaaccaaataattaaaaaaaaacacttttcataaaaagaaaattatatgcacacccccaccccctgccccttcctcatcaaatgtgtcaaaagaagtattttttttgtcattttatgaatgttgtgagataagaacgtttttattctaataaatcgatatttgaatgcttgatttgatgagtgtgataatgtatttgcttgatctttttcgggtttttttctcctttttcgctggatttttgggtcaatgcaagtaaaaaactgttgctttagtacataaatacggagtctcaatctttctactaaaacttatatagcttatctatttgaggacatacacgaggtgtttaaaatgaacgaaacttagttaaggtgtctaagtaaaaattagtgccaactttagaaggtcaccgatgggtttggcctaaaaaattacgacctttgtctatatatctatatattacataatcttggatgaaaaatgctcaactgatcatcgatcttttaacttaacaagatgtggaagttgaagattgcaaaaggacaagatgatcgatacttgtatagtacaaataactatattggacgacaaatatgggagttcgatccaaatgctggaacaatagaagaacaagccaaaatcgaagaggcacgtcaacattattggaacaatcgttacaaagttaagcctaatagtgatcttctttggagaatgcaggtcagtaactataagggtcgtctcgatcataaggtcacaaattttcgatcatttttggttgatgttttgtcatgtgtaggctataaagtttgggacaaatatttgattttttaaaaatgatttgtatctataaattataaaaattattaaaaacacctataaatttgtattatgaaataaattgatttaaacgtagtcgagacgttgattgagtgtcttatgtatgttatagctttaactgagtggaaagttaaaatatacgcctaaattatttcaaagtctaatttttcgaattctatatctaacttatatacatttatattagaaaaatgaatgttataactttttcttattgagatattgcccttatacatattaaaagaaatataaccaattctttgatacaacaaagtccttgtaagaagcacaagtgtcttaaataaatattttgaagaatgtcaaagtgtcaagaggcatagaacaggttgtgtgtccaaagattttcgtatgctatagtccatggatttttgatgattcaaaattttgccgtgttttttatctaaatttttcatggacgttcgttaggaccttagtaatagagctagtggtctcgacgagcaaatcgacccactttcaagttcaaacgagccccaaagcaggtaaacccccattttgtcgatttcgtgtgttatagttaatgaatttttggtgattcgaaatttcatcgtgtttttctttaaaattttcatggacgtccgttaagaccttagtaatagagttagttggtcccgacgggcaaatcggcccattttcaaattcaaacgacaaggttgagttgaacaaactcaaggactttgtggagtttctgatctatttcatcagatttgcacacttacattgacagaaaatcaaaacacgatcaggaggtttgagagagtttttgatattaacttggagagtggtgcgagttgagtgaaaagttgtaagattgtataacaagagtctttatttacaatggagtgttgtgagtaggtttcgtttaacaaatttgaagtacttaaacaactatagaagacttaaaccttggtgttttggtttggataactagaaattagagtttataattacgcagtgatatttgtgcagtttttgagagagaagaatttcaaacagagaattggagcagtaaaagtagaagaaggagaagaaattagccatgaaattgctacaattgcattgcgtagagctgttcatttcttctcagctttacaggctactgatggacattggcctgctgaaaatgccggacttctcttctttcttccacctctcgtaagttttttttcatttgaattcgtaaacttcttttgataaaatgggtgaactaaagccaaaattgtttcaggttatgtgtatgtatatcactgggcatcttaatactgtatttccagctgaacatcgaaaggaaattcttcgttatatatactgtcaccaggtatatgatttaatgcatcaaattcttggaatttatgttttaagttgccagcataatacataaacgtgctcttttagttcggttttagctgagcttagtgcggcgagtatgatataaagttgaataagtatacaaacattctacgtgacaattcgcatgacatatactcgaactaatgtgtggcgttctacatgtattatggcacgtaggatgtttgtgtccacttgtttggttttatacaagtttaagtgtctatttgtacacactcaaagttggagggcgtatatatcagctgatgccaagttaaaatgcatatttctgtattatgtcttaaaaaaaaagctctgagttactgtggaattctgtattattcagaatgaagatggcggatgggggttgcatatagaaggtcacagtacaatgttttgtacagctctgagttatatttgcatgaggatccttggagaaggaccagacggtggcgaaagaaatgcgtgtgctagagcgaggaaatggattcttgatcatggtagcgttactgcgattccttcttcggggaaaatatggctctcggtatgattaattatcctagtaacttttttagaagaatatatatactatagtaggatttaagttatatacacttgtagtgtacaaaatttttacagtatcaatgtgtaatgtttattaatctgtttgagtttgatggattcaacgtttaggttcttatcattgaacacattgcatttttgaatttataggttcaaaacttagtagtttgcttaaattctagggtcaagaacattaggcttgttgaacttattcgttatttactacatacattcctctgcaattatggtggatggttggttaattgacgttatcatttagatgacttgattctgcgaaagacactatctttaaggatatttcacccggtacaactgtatcacatattcacaagttcttccggtataaaactaggagccaacaatattgctaataaatcttccttcttattcgttcagattcttggagcttttgagtggttaggaaccaatccaattcctcccgagttttatattcttccttcttttcttccaatgcatccaggtaggccaattttttttattgctttatgcatacacatcctaaaattgtcagttcattttgttgtaaatattaattttgaagatttgctatgcaatgttttctggttgtttctatcctctgttagtgtcgatatattgatcttccaagtattttgactcttaatttaaatgtagcaaaaatgtggtgttacagtcgaatattttacatgccactgtcttatctctatggtaagagatttgttggtccaatcacacctctcattttgcaattgagggaagagttatatgatcaaccatacaatgaaatcaactggaaaagagtacgccatctatgtgcaaaggtatgactatcgagggtcgtttgattactgatattagaaaaataatctcgacataaatttcgatataatttctccctctaaaactttgtctacggatcaaatgagtccctagtgttgaagttagtatagatatcatactattttctacatatgatcaaagacaaacaaacttgttgctgcaggaggatctctattaccctcatccactggttcaggacttgatgtgggacactctctactttttttacgagcctttattgactcgttggcctttcaacaagctgagaaataaagctcttgaggttacgatgaaacatcttcattatgaagacgagaatagtcgatacatcactgttcaatgtgtcaataaagtacgacaactttttcatttttgatagattacaaaatacctgcattttcttaacaacttcaatcgtttgacacttatgttacacgtgtatggtgtaggtactgtgtatgcttgcttgttgggttgaggatccaaacggcagttatttcaaaaaacatcttgctcgggttccagattatttatgggtagctgaagatgggatgaaaatgcaggttggtagatataaattattttctataagcaaggacattatactattccacgaaccacaatgatttacgaaataggatctcttttacacgtaatacatgtttgtaaaatagaaggctagctccttttcctattgtgaagtgatgcaaacaactgttactgaaattgcggagttttgttgttgaattgcagaatagcggtagtcaatcatgggatactagttttgctattcaagcactattggccagtgagatgaacgatgagatatcagatactcttagaaaaggacacgacttcataaaaaaatctcaggttgtatgttaaatcatgagcataactgctctttcaagatcttattaatgtaagtgacttctcgctaggtaaaggataatccttctggtgattttagagggatgtatcggcatatttcaaaaggatcgtggactttttcagatcaagattatggatggcagtcatctgataccactgccgaagcattaagggtatctaactactcaatgaaataagattccaacttcttgtaattactgaaaagaagtaagatgataatgctaatagatttgcctgatgacttgttaattctgtttcactagtgctgcctttttttctctacaatgccatctaaattagttggtgaggcaatggaacctgcacgactgtatgactcggtgaacgttattctttccttacaggtaaaaaaaatgagaaatgtacatgttgagtgcattttaatacatgacttatatcttacgccgaatagaaaatccaatacatgactacctgcgttattaaattcatcttttctaaatttagtcactcgtcatttggctcatttctagactctagttatatcaaattccattttaaataaaattgcagagcaaaaatggcggtttagcagcatgggagcctgcagggacctcaaagtatttggaggtaagtaaaataattgttttggtttgagattcatttaatggttgttaacaacagtcaacttcttctatagtaatccaaaatctaaaaatttaactttcttgttatgttatattccagttgctcaatcctactgaaatatttgaggacgtcgtcatcgagcatgagtaacattttgtccctagtttctccattccccataagccttttccccattgttaggctccatttcacagcttcacactgattacatgtcaatccaggtatgttgagtgcactggctcatcaatacaagcacttgttcattttaagaagctataccctggacaccgaaccacggagatcgacaatttcattgataatgctgttaaatatattgaaaatgtacagaggcctgatggttcatggtatatgatatttctcttgcgagtccttgaactttctgctctatcaatgcatatgatatgattaactatgtcaacaggtatggtttctggggtgtgtgctttacatatgcttcctggtttgctcttggagggcttgctgcagcaggcaagagttacagcaactctgcagctgttcgtaaaggcgttgactttctgctacaaagacaaaggtctgatggtggttggggagaaagctaccattcttgtcccgacaaggtaatgtaaaagaaacaaaagccacgtaaaacaacaataactacagcaatgtctcaatttgtgcattgtaaggtcagttatatgctatgtttctccgtacttcacttggtgcgcatcacattctccaaaaagtagtgcattcttggacaatctgcaacaacacttttggggagtccttgcaacatagtaggttatatgaactctgcacatgcatcctactacatttggaatcgtttcattccaatactgaatattttaagtctttaagttttctattggcatgcatataaatttaccgctaaagaagcgtatgaagatgactggaaaatataacgatgatttagctttgtatttgatcaaacaggtatatagagagcttgaaacagaacactcgaatcttgtacaaactgcatgggcattgatgggattgattcactctggccaggtgaattccttgttactagtagtaaaagcataatacataaagaaacactcaaactcggtctcagttgtcaagtaagcactacaactatgtgagtgcacatctacgcacttcaactcgtttccattgtgtcatttgaacacttcaacttataaaatgatcatctagacacgtcgagaatatatgtaccatgtcagtgtcggagtgacaatgagtcacaatactttcctatttggacaggctgatagagatccaaaacccctccaccgtgcggcaaggcttttgattaattctcagatggaagatggtgacttcccacaacaggtaattgcactgtaatttacaagataatcataggagaagattcttcaactgttaaaagaacatgctgttaagtattggatgaaatagactgggaaaaaacgaaacaaccatagatgattcatttagtcgaattggtcatatttgattgatattgattagtcgagaaaagctcaaatatgtcatggaactttcagtgaagagtggcgggaatgtggactttgtgtcccaatgatgacataaacaaagcctaactattaacctatggcatgactgagccatttctgaaagttcgataacatgcactgagcttttttcgtcgattagttaaatatgaagaagtttttaatctgacttcataaatatgtgcaggaaataactggagctttcatgaagaattgcatgttgcactatgcaacatttagaaatatatttccattgtggggtttggctgaataccgcaaaaatgtttcagtatcattaaaaaacagctaaagatatattacattagttatatcttaagaaataaataagggaaaatgtacaagtacaacctcaatctatgtccgaaatttcagagacacacttatattattttaaggtcctattttccccctgaacttattttatatgcaattttctatcccttttcggcttacgtgatactagcttgaaaaaaaaatcaaccatagttggacccacaagatagtgccacgtatgccgaaaaggtaataaaataagttcaggaggtaatagtatcttagtatagtataagtgtatctctgaaatttcggacatagattgagggactacttgtgcattatcccaataaataatgaagtttattgtttcttgtgttaatgcaagtgtcatgtaacaatgtccaaaatcatagcctaaactgttgttaaatatttataatatgtgaataatgaaaattacaaaaaaaaaaaaaagcatgttaaaaaaatatttttcgaaaaatatttttttttcataaatcaaacaataaaataaaaaattcggaaatgagcgaccaaaaaagcaattttttaatttatttttgtgttgacaaaaaaaaaagaacaagaaattgctaaagaaaaaacagttctttatttgtccactatattaagaatacattttatttttcacttttagcatatcaaactttttttcttccacattttttcctagttacttattccaaatcatttttcaagatgtatgattatatattcattaatttaaatattggggtagatatacttatattaagaggcctacaaaaattaaaatgaacaaataaaaacgatcaaaataaattacaaacatacatggggcccacatttgcgaagtaacatgttttgttgctatatgtgtataagcaagcaagtattgacagagtgctaggggcctatattcacatactagatatgaaaagattcgttaacgaatttaatattacggtctttaattgtatttatacgtgattaattatatttttaaataataaattaatttataatattttcaggtttaacacaacatagagtgatatggccattaaaaacttatatgtaatttaattaagttgccacaaactaacattagaagagtttaatatttattttagagaaaatacataattattctatcaaacttacacaccttaaaatcgatgaaaaaccgacacatgacaacgtcaaaccaaattgaaaaagagacccacatttcttcttccctaaaacaccaatattgacggttattgagagcttttcccaacaacaaaagttggttctttattacgcgaaataactttttttattgtatactctatcatcaagttttaataacccatcttaaggtcttttattagaaaaattcacttaacaatgacataatacttggccaatgtaacacctaatttacctgttatcaaagaaatttaaacctattttcgttaatgtagtttcccaagataaaaaatctcaatgaatttactcgtataaattcagaaaaaaagtaaaatttacttataggactcaaagctaatcttgacttgggaatagaaacaaagctaaaaaggactaaactatatctttggaggaaaaaaattgaaatcaaacaccaaataaacaagtaattaaaaaaaatattatttcatttgggtgaaacacatgcacgggttatgaaattgcaaatatttttaaaatggtgtatttagaattgttgcaactcatttgacggggaaatagaattctcacaaactttagatctctacttaaaaaaggatacaatcgtacaaatttaatggaataattatgcattttctctttattttaacattatatcacacacaaatattgaattaaataatattatgtgttggatatgagatatgttacattctttaattttattagcctcatcaacaagtcgtaaacttccgggatgtttcgattcggacagatgtacataattaaatgagacgacatactttatattgttagcataattatagctaatagacgtttgtgaacatgcgcatactctccaaatctaaacaaaaaaatgtctaatatggatactttatcgtgtgtttggatgttcacttggaacaagtcgtaattcgaatatcaaaataaaatttatcggtgaatttaatacgctatttatatattaagtcaaataattaattcatcttattaaataataataaaataccatttttccctaacatgcaacaatatttctatttaattttttgtatatcacaaaataataaaattccgaaaaaaaactacgtttatgatgtctatcttaaaagatgaaaataaaatttataaaataatattttaactttgttagcatgataaatattattttaaattttgatagtgattagatatttgtgtcattaatgtctccagtgaaaaatcattacatgtaatttcaaatcataaattacatttgtaagctttctatctacattttatcgataaagtcttttagtaaacgttttttttttttaaaatgaattatagttatatataaaataagtttgaaattttatttttatttaatcaaataaaaagaaagtattactattacatttgattgaacataaaacttgtggaaatttatttttgtcaattatagtatgttcatgacatttactgaagcttgaatgacaaatattgtccacaaattatgaatgaaatgataaggacttgacattttaaatttaggataataaaattaaaatgatataaagagaactcatcgtacaatataataaattaataataatgacatacaaccaacaactaaaactttatttatttatttttttatataaataattgcatagatcactcatttttttggcacaaacataatttataatttcttctctttcagcctactaaacttggtgagtttttactctctttcatttttttttaagaaaacatttttgtgatcacttcatctttagtattttgcttgatatattattgaacatttagagagagatatttcgtaggtaagtatttattactttcaattttaagattgtaaattttatattagagaaagtgcattaaatcatttatgaacttgacagcaaaatatattttactattttaactatagtcataatacataaatatgttcctttaacttagcttcaaatcacatttatgcctttcaactttggatgtgcacaaatagatatttaaatttgtataaaattaaacaaatagacacacatgttctgcatgtcattttttgtcctacgtgtattttgccatgtaggactcacgtatttatttatttaaaagttggataattaaaatgtctgtttttgcattatgaaagttagaggtcaaagttaaaatttaaaattaagtttaactcatcgtacgatataataaattaataatagtgacatacatgatacaattaactactaaaaatcctaatttttttttataaaaaaaaatccatataattgtttacaagaatattcaattgatcatctttggttattgtatctaactatggtcctttagataattaaattcacataaattacctatgaaattctcattgatatcactaaattgttatccttttgtaagtagaagaagatattaaaaag

SEQ ID NO: 4 - DNA, 8938 bp, Solanum lycopersicum
  source 1..8938: mol_type = genomic DNA
atgtggaagttgaagattgcaaaaggacaagatgatcgatacttgtatagtacaaataactatattggacgacaaatatgggagttcgatccaaatgctggaacaatagaagaacaagccaaaatcgaagaggcacgtcaacattattggaacaatcgttacaaagttaagcctaatagtgatcttctttggagaatgcaggtcagtaactataagggtcgtctcgatcataaggccacaaattttccatcatttttggttgaagttttgtcatgtgtaagctatataaaaagtattagctttagtttgacataatacataattatgccatttaacttggttttgaatcacatttatgtcctttaactttgggtgtgttcaagtagacagtaaaacttgtataaagttgaacaaatagatacacatgaactttgggtgtgttcaagtagacagtaaaacttgtataaagttgaacaaatagatacacatgttctatatgtaatcctacatatcattttttgtcctatgtggtgtcctacgtatattttgtcaggtaggactcatgtgtttatttatttaatagttagatagttaaagtgtctatttgtgaattatgaaaattggaggtcgaagttaaaatttgaagccaagtttatggttcaatatatgtattaattaaaacttgagaaaaaatagacacattgaattaatttattttttaataagtcatctctgattcaaataatgacccatttattgattcatgccattttaaaatgcttagagaattagttttatggcttaatacataaacagatccctaaacttgttgaatttgttctttcaggtacttcaactatgttatttctctattgaatcattgaaccaccataatttgttgcttttaaacaccgttggctgatgtagaacaagattttatgaagggtattgatagagaatacatatgttgtttcataactcattaatccaattgatagagaaaatattcgagaataattgtgttttacttcaagttatttgaacacattagaaaacaactgaaactaccacacagtttgtcttcattcctttaatcaaaatcattacaatacaaacacaattgaagacatttacaatagaaaaaccgatcaaaatcggttaatagtatttaaaaggaacaaattatggatggttcaatgattcaatagaaaaatgacgcagttgagataactaagagaaaaaatcaacaattttagaaatctatttatgagttcagcctagttttatagactatttataaagccataatacatacacatatcatctaatttaatcttatcttatattgcctcttatatttatgttattaactttaaatgtatgcaggacttaaacttgtataaaattaaataaacagattgatatgtcctatctgatattgtaggcgatgtcatatatgtgttggcataatacataaatatgccctttaacttggcttcggattatatttatgcctttcaactttgggtacgcacaagtagacacttaaacttgtataaagttgaacaaataaacacacatgtcctacatgtcattttttgtcctacgtggtgtcttacgtgtattgtgtcatataggactcatttgtttatttatttaaaagttggatagtgaaattgtttatttgtgcattacaaaagttggaggtcaaagttaaaattcgaagtcaagtttaggatccaatatatgtattatgcgtatttgactcatgtgtctaaagggtctgatatacccctcaactttgtcatttagagttgatataccccttgttatgaaagtggctcatatatacccctacttgtaaacaaatgactcacatatacccttttcctctaacggaaatgaaaaaaataataattttcaatctatatttttattattttttttccaaaaaaatataatcccatatgagtaaatttaatcctcgtcaaacatatttttttgactttttttttgtttcaatgactaatttataattattattttgataatcaaatttatttatgtttcactaatattcttgtaaaacttattgtagatgaccaaatttttttttgaatacgaaattaaattacaatacacacaaaaaatagtttattttttttctttaaactaaagaatgaaagaaaaaaataaaataagaataagaaactcaaataattataataaaagaagtcaaaaaataatttatgtatgaaataaaaataaaaatataccttgaacttttatagaagaatcatatataccactaaataatttctataaaaattataagtaataaatataaatttaaaactaattatttaaaatttcgttgaatgaagggtatatgtgaaccattttgtaacgacaggggtatatgtgagccgtttgtataacagtaaaagtatatatgagtcacttttataacgagcggtgtatcagatccaaataataaagttaaggaaatatcagaccctttgttatataagttaaaaagtgaaataatgccaagttaaaaggacaaattgacggataatacaattattgtgcagtttttgagagagaagaatttcaaacagagaattagagcagtaaaagtagaagaaggagaagaaattagccatgaaattgctacagttgcattgcgtagagctgttcatttcttctcagctttacaggctactgatggacattggcctgctgaaagtgctggtcctctcttctttcttccacctctcgtaagttttttttgtttgaatttgtgaacttctttcgataaaatgggtgaactaaagccaaaattgtttcaggttatgtgtatgtatatcactgggcatcttaatactgtatttccagctgaacatcggaaggaaattcttcgttatatatactgtcaccaggtatatgatggagtgtcacaatgaatatactagtgcaaataaagttcattctgaattactgattgttatatcctgtattaaggttagtcaaaaaatattgaaacaaaaataagatgaaacggacagaaaaacttttaattcgaatccaccgaaaccattgtgttttattaagaaatttaattacctaactgtattcgagattgcagattaatttctcccaagataaaagtttgaagtagcggtacatcaaacttcaattacttcagtgaacgtaagaacagtaacaagagcacaagcggaacctcaaactttccataacttcagtgaacgtatgaacagtaacaagagcgcacatagactcagtcgattgaatttagtttttgagagaaaatgtctgcaaaagtagcgctacttaggaaaaattttcaatgcttgaaaaaatgaaaaggacctccattttataatcattttcagcaagaaacgtgtctgttcaaaccaaaccagttttatccaaaaaaagctgtcatgcaatgacggcgcgaggggcaccttcttcttgaccctttaagaagtaaaagaagtgtttcctaatataaggacaacaattttctttcttccaccagtatgggagaaatgacttttcattttccttccaaaattggttccctcacttttccatttttctcttctcttttcccattcacattttgctaaaacccaacactgatgacattctgttttattcagaatgaagatggtggatggggtttgcacatagaaggtcacagtactatgttctgtacagctatgagttacatttgcatgaggatccttggagaaggaccagaaggcggtgttaataatgcgtgtgctagagcaaggaaatggattcttgatcatggtagtgtcatcgccattccttcttggggaaaaacatggctctcggtatgactagtatttaacttatatacacgatagcatacaaaattatatattttaacttgttataacagatagtatacaaaattatgtattactatctgttataacaagtactatactgacaagtgctaatgtacaaaaaaattcacattgttagtgaattttaacctgttgtaattatcttttagacggcttgaatcatttcatatattcaagtaaatttcgagtgtcatgtatgtcttcgtgctatgtttacaattactctttgaagatctgacatgggtgcggcaatatttttggaggatccgagcaaccttgtcttcttgtgctgagaaatttccccttctttcaatcagattcttggagctttcgaatggataggaaccaatccaatgccacctgagttttggattcttccatcttttcttcccgtgcatccaggttggttaaacgtttttattcctcgtgcatgtcatccaggttggttaatatttttattcctcgttatctaactgcagcaaaaatgtggtgttactgccgaacggtctacatgccaatgtcttatctctatgggaagagatttgttggtccaatcacacctcttattttgaaattgagggaagagctgtatgatcagacatatgatgaaattaactggaaaaaagtgcgccatgtatgtgctaaggtatgactctcaaaggttgtttgattaccaagataagaaaaacaatgccgacatgaagtttggtgttatttgtccctgtataaccttgtctacggaccaaacgagtcctcagtgttgaagttagagtctacatcataccatcttctacaaatgctcaaacatgaacttgttgctgcaggaggatctttattacccgcatccttttgttcaagatttgatgtgggatagtctctacatatgtaccgagcctctattgactcgttggcctttcaacaagttgagaaataaagctcttgaagttaccatgaaacacatacactatgaagacgagaatagtcgatacatcaccatgggatgtgtggaaaaagtacgaaactaactcatttttgacagactaataaattacctgcattctcttaatgactacaatagttcaattgttagagaacactgaatgtaacacttgcatgatgtaggtattgagtatgctcgcttgttgggttgaggatcccaatggcgatcatttcaaaaaacatcttgctaggatcccagattttttatgggtagctgaagacggaatgaaaatgcaggttggtagatcggaatagttctatttcacaagccacaatgagttaaaaaaagaagcgcatttacaattaacgtgttgtaatataaaaaatctagccccctttcctagtttgaactttgaagtgatgcaaacatttgttactgacattagcgagtatttgttgttgaattgcagggttgtggtagtcaatcatgggatgctagtcttgctattcaagcgctattggccagtgagatgaatgatgagatatcagatactcttaaaaatggacacgactttataaaacaatctcaggtttggatgtttagatggtgagcatatcaactctttcaaatagtggaccttattaatggaagtaactatttgcaggtgaaggacaatccttctggtgattttaaagtgatgtatcggcatatctcaaaaggatcatgggcttttgcagatcaagatctcggatggcaagtatctgattgcactgccgaagcgttaaaggtacctaacttttcaatgaaatgagatttcaacttgtaattgttgaaaaatggtattatatttttgtttttctagtgctagtaaattttcctgatattttcttatttttgtttatctagtgctgccttctcttctctacaatgcctcctgaaatagttggtgaggcaatggatccagtgcgactgtatgactcggtgaatgttattctttcattacaggtagacactgaatgattaaatcattaagtcatttaatctgaaagttgttttctgcttgtagaatgattccaagagaagcgtatccaatacatggttacctgtgaattgtgaaatgaattccttttttctgaatttagtcattcgtcatttggctcatttctagtaatgatgagatctattttaaataaactgcagagcaagaatgggggtttatcagcgtgggaacctgcaggggccccagagtatttggaggtaacttctctgccaagtaaaataatcgttcgaattttgagattcatgatatagtaagtttatatatcttcttcatttcaacgttgttgaaaaccttcgacctcttctataatctttcctgaacctgaataagtatttttcatggttgttatatttctcagttgttaaatcctactgaatttttcgaggatattgtgattgagcacgagtaagattcgtttctctctctccctttagtttttttcccacaattccaagtctccattttactgcttcacactgattcaatgtcattccaggcatgttgagtgcactagctcggcaatccaagcacttgttcgttttaagaagctataccctggacaccgaaccacggaggttgacaattttattaataatggtgttaaatatattgaagatgtacaggagcctgacggttcatggtatatgatatttctctcgtgagcccttgaacttgcaaattcctttctgctttatcgattatgacatgattaatcgtgtcaacaggtatggtaactggggcgtgtgcttcatatacgcttcctggtttgctcttggagggcttgctgctgtaggcttgtcgtacagcaactgtgcagctgttcgtaaaagcgtagaatttcttttaagaacacaaaggtctgatggtggttggggagaaagctaccgttcttgtcctgacaaggtaatgtaacagcaaaaaaggccacgtaaaggtcggttatatgaacttttgcttcatttggatcttttcgaggaggaaatataaagatgaatgttcacaaagacaactgtgactcagctttgtatttgatcaaacaggtatatcgagagcttgaaacagaacactcgaatcttgtacaaactgcatgggcattgatgggattgattcactctggccaggttaataccttgttactagtagtaacatgttaaattttttcaccatcgtagtaaaaagtttcttggacaggttgagagagatccgaggcctctccaccgtgcagcaaagctgttaattaattctcagatggaagatggtgacttcccacaacaggtaattactccacgaatttatctgtgttcagtatttggatgaatttaaacgaaacgaacatagatgattcatatatatagtctgactggaactagtttagaatctagtcgtatttgattgatattgattaataaatataaagaaacaagctctatcaaactggcaaaactgatacaatacgtgcgtccactatcagtcaacgcaccaggtcccttgacacactcaaaataagaatgaagagtaaataatgacagaaaacaacacaagaagtttacgtggaaacgtccttgctcaagggagtaaaatcacgatctgtcacacaggattttcaacggtcttcactaatcttcacaaacaaaagtgaaacccgatttgcttgaagagccttagcagatgacactctgtccactaagatatcaccactagacaacttaggattcaactaagcaacatgcaggttgctcactaaatcaccatgctccggatgacttagagtttaactaagcaacacacaaggctgcccactaaattagttggactcacctaatttagactttttcaacaaaccaaaatatctgaatcctttatacatttcgaaacagatttacaatgcatgtgcagaaataatcctaagacaactaaccttccagctctttccgattcgagttgatcttaaggatgcttcttcacttcttttgtcagccttgcaagagttcttgagatgtttttcaagttgcaaaaactaaggttgtgactattggaacatcatctttatgtagaatagtcacaaaaccatgtcattggccgaggtttctgccactattggatttgtgcaccaactttttgtattttttccaacctgacagtttgtgtgtatatttccactcgggggcatgtctctttacgaggtccctgagtttgttatatcatcaaaactacaaataacaaaaacaatatttaatagaacagagatttgttaaaaccagttttatcaaattattgtttttatctaactttataaatatgtgcaggagataactggtgttttcttgaggaattgcatgatgcactatgcattatataggaatatatttccattgtggggtttggctgaataccgcaggaatgtcttagtaccattaaaacacaactacatatagagaatgcaatttgggccttccgcggattccgtatatagcgggagcttagtgaatcgagttgtccgttgtttgttttttgtgttaatgtaagtgatcacccaagtagtggttagatatgactatgatcgacctgttaggttgtggagcctgattaatatttgttatactgcaatatttattctccatttattctctataacctaaaatactctgaattattaatatatatacccaccgccgtggaagtttactcacggggtgttaccacgaaatattggtttctctctttctctctctagatctctcatctctttctcttgaaagttcttgtgttctaaattcatcatgtgtgtgtggattcaatgctaatacgacccttaatcaaatggttcggattcgagcctcgaggatggaaagaatcttgatatgatatgcttcctcctttagggttctacataggggatctacatgaatccaatgtatacattactcatgaatctactaaacatctattgatatttgtttgtaagctcaattattattttcgttttagtattatttagttatatcgtagtttctt

SEQ ID NO: 5 - DNA, 4804 bp, Solanum lycopersicum
  source 1..4804: mol_type = genomic DNA
aatacatataaaataattctatattttagatttgatatatttataccttatatgtggtaaagtcgtaacaaccttcctcaccatatgtgaactgggccggtgataatgataatgaaaataagaagataaatatatcactgatgattacaaagtagtgagtgattttagcaatggaaatctggtttatcgttgttgttactctctgtgtagctttctggttgaaatccatttgtaatattattgtctctccaaattccaataccaagagaaacctcccgccgggaccctacagttttccggtgatcggtagtttattatgggccaaacggaccttctccgacttggaacccatcctccgtgaccttaaagctaagtatggtccgctgattaccctaaatattgggtctcgttcagccatattcgtagctagccactccttagcctaccaagctttagtccagcaaggtgctattttctctaaccggccaaaggctgggcctaccagtgcagtcgtcaatagtaaccagcggaacatcagctccgccccttacggccctatatggcgtctactccgtcgaaatttgacttcagaaatactccacccatctcgtatcaagtcatattccaaggccagatcttgggtgctgggtatcctcattcaacagctccgtaacgcccaagttgattctgtgaagttaattgatcattttcagtatgcaatgttctgtcttcttgtattgatgtgttttggggataaacttgaggaaactcaaatccgacaaattgaaaatatacagcgcaagttgctcctgggtttcacacgattcaatataataaatatatttcctagagttggaaaaataatttttaggaatcgctggaaggaactcattgaactacgacaagagcaagagagtatcatcattcctttgattgaagctcgaagcagggccagagaacaaaggaccgagcacggtgatgaatttgtggtggcttatgtggatacactgttgaatttggagttcccagaggaaaaaaggaatctcaatcatggagaaatcgtaacgctctgtagtgaattcctaagcgccggaactgatacgacgtccactgccttacagtggattatggccaacttggtcaaaaacccttccattcaggaaaaactataccaagaaattgccactgtagtgggagaaaaacagagcaagttgacagatgaagaggtggtgaaggaggaggatttgcagaaaatgccctacttgaaagcagtgatattagaaggtcttaggcggcacccacctggtcactttgtgctgccacacacagtgacggaggaagtagaactgaacggctacgtcatcccaaaggatgtcaccatcaatttcatggttgcagagatgggtctagacccaaaggtgtgggaggatcccttggagtttagaccagagaggttcttagtggaaggatcagataacgaagggtttgatataacagggagtagagagatcaagatgatgccattcggcgcagggaggagaatatgtcctgcctatgcattggctatgcttcacttggaattctttgtggctaatttgatttggcattttcaatggaagcctgtggaaggagatgatgttgatctcacagagattctagaattcaccgtagttatgaagaacccattacgagctcgtatctgccccagagttaactccgtttgaatttggtaattgcttctttttgtttgcatcattctctgttgatggactttccccccgctggaagttagtgggaatatgattattaatgccttgctgcaatatcagcttagtagtttttattggctaaaacaagattcaagctactaagctgatattgcagtaaggcaaaagttaaactggctgttccttgcttatatcttgtccatttggcagatagtagttcaatttgtactttcctgaatcttgtgtaaagaagtgatgcctgttcactagaattatactattccctccattccactttgttttgactctgttccctcattaatcaattaaaaaaatacatttctatatttagtcacaaatgtttaaatgttagatttagatgtttttcgttcttaaattcctctataagaatgctagttcctatagttcacatattatgattacggctattaatacgagggaatacactccttttcgtcgtcccattgggttctcggcagtcgcatgaaaattgggacattttagtctttctattatattttatcctgacatcttctgtactttttgttctggaagcaaagggaagagctattgtgggagataaatagtcttattctttgcgctgtgacctattcttctagctatgactgacctatgcttttgtggggatttcaagtatggctcctcagctttgtatattataccaaatacatcatgttaacattatctgttgaattagccttgtgtgttggccgttgggaggtagatatattcttctatttgcggtcgtgagctatatccaggaacgcaacacttgtttctccatgtacattcatgatggctgtaaagtttatctgatcaactaattttactttgctgccttttttttcttatttgatctttaacatgtgaggctatatcagaaagtttcctaacagcttagcgtgacttcacaacttctactttaaatctggaatttcctttcttttgctttatttttcttcgtgcttttggagttttgttcttctgctatccatcttgtcgtttctatgcctgcttgtggtaagtgaatttgttgcgcttcctctttctccctccttcagatttctattgctgcgttgattgtgtgcccacacgacttctctgtaacaacataaaatttataaaagggaaaagatcaatttaaagtagagacacctttgtgctaactttgtttgacaaataagaggaataagaaggtcttggtcgtctttctgtgttgttgacaactccactaaactgtggtttttttgtcttcatattatatattttacacagctagattttgtacttctctggtggaggatgcgtaaaatttttcactgaatttctttccgctaggtttgtgctgtcgtttctttcctttaaaactgtctaacatcaaaacttcaagggtatagattcagctctcctgtagaatcaaccttgtaatctgttcatgaattctctgtttctgttgggtaactcttgtgtttctctccggccatatttaataggcacatgcaacaatacttgcctggtacattgtagcttttacattcttcctattggcattcagaacagcccactggatctcctcttgccatttcattgtagacttcctttcaatgctcaatatcacaaaggggacatagtttactctcaattacccccactgcatgaacctgtcctttgtgtatagtctgccatgtaggatcaacagtagtaggaaaattcattaggtgctccaatgttattgcaagtcatcctccactacggtactttcaggaaatcccctcccaaatattttttagacacaatactatgactaatcactaaatgtatattagcagtattcataaggtacaaagctcaattgtttacaacctgcaatggaatgccaacattattttctttttctttggattgaattgtttcttacaatattgagaatatgatagaccaattgtttgtttatactaattgtaatttaaaactttacctccaacaaagtaaagtgtaggtcattatttctgtacgatcatttatttactttattcttactaattggagtctgactctactttttggaactatgaatggcactccataaggcaccaacatcttttgaccacgctctgcttcaaaagagaaatggatgagaatggtatttgctcctcaagctagttagaatttagaacttagtgacttgtcttggatcccttctaggctagtgcacaataattttgatggtagtcacaatttaagattgagcttagatggctagttcagtaggaaagccttatctttttgttgctctatttcttgcttgttcctattatgaatgtgttgcttcctatgtgaaaatattaaaatttgtgggtttcttcttcttttacttgggtttgatacacttgagccaagggtctttctgaaacaacctcttcacgaggtagtggttaggtctgcgtatactctatatactccccagagcccactttgtgagatttcactgggtatgttattcttgttgttgtggtgtttctggtagttgttatatctgctttatattctgttctatcctcccttctttcctagtagggagggattctttgttaattttgtttattgattataaatccatgtgttacaggaaacagcaaaagtgaatatgataagcaataagctgtcgaatgttccactgatgcaatggtgggaaataatcatagctggaataccgcattagaatcatttgggcacacttttactcttgtatttgtaaaacttatttttgaaaaacttattctttagccgagggtctgtgggaaaccacatctatacatcacacaaggtaggggtaaggtctacgtacaactacttgtgagattacaatgagtatgttgttgttgcctgatggagcagtgtccatctttctcaaaagttataacagtggcttgtaacctatttttgtatagattgctattacaaaaatgccgtgtttttttttatagttctacttgttattgtttttatgtttgtgttgtgcaactttgtggatg

SEQ ID NO: 6 - DNA, 3136 bp, Solanum lycopersicum
  source 1..3136: mol_type = genomic DNA
atgaaataataaaacttcatcaacaagaagtactgtttgttttttgttatttatttatttatgtttgttattgatttgaagaagaaagaaaaaacttctctccaattgtctgcttctagctccggcgctagccagccatccaccacaatgtcactctccatttttctttttcctccatttatagagccttcgtgctcgtctaaaagtgcccagatctggtccattttgtgttttagccgctgcttcaacaatgactgtgaagccacgccacaccacaccacaccacatctcccttgttaatgtcaatccaatcaacaaaattattactcccacaaaacttcattttttcccgataaatttcattgatgggcttttttcattttatttttctcctatcctgtagtatttcttggttgctgcattcttgataaaatgaagaatcatacatgaggtaacacaatttgcagaaattatcagcgcagtagcttcaaatgattttattttttttaaaaaattgttatgtaaaatgagaaattattctaggttgggtcaatcggatcaattttcattgatttaatgaggggtacagctgataaatttgttaacggtaggggttaagtataattttattttaactaataaatgaaagttgaaagtacttttgaattattttcctttttttatttagtcatggcacaaattaactggtggaattatccgtacggaatactggtaaacttcataaaaatttactccatgatactatcaaataatggtccaaggatacaataattaagcaaggaaaaaaagttgttatgtggtgatcgtctcctttagtttctgtggtgaagcttatctgaatatatctctttacttaattcccattttctcctaattactaaatttagaaaaaatggagtttccttggtattctgttgttgttcctctgattgttttccttttctttcttcataactgcttatttaccatttttaatacgagtaacaaaaaattaccaccatctccaagaaagctcccaatcataggaaatttgtatcaacttgggttgcatcctcaccgttctctctataaactatcgaaaaaatatggtccagtcatgctacttcacttgggcagtaaaccagttctggttgcttcctctgttgatgctgctcgcgatatcttgaaaactcatgatctcgtatggtcaaccagaccaaaatctagcatcgctgatggactcctttatggatccaaaggtgtggcctttagtaattatagtgaatattggagacaagttagaagtgtcatcgtgcttcaccttctcagcaacaaaagagtacaatcttttcgtgatatcagagaagaagaagtatcaaacatgattgatgaaatcaggaaaaggagtactagttcttcgaattcagtgatagatatgagagatgttttaagttgtatgactaataatataatcaaccgagtgacaataggaaggacatacaatgaaggggaaagtggaagagctgttaaggctctattagaagatcttcttgcacttttaggtacttttaacattggggagtatattccatggcttaaatggctcaataaaatcaatggtcttgacaacagagtgaagaaagtagctaaagatttggatgcatttttagacagcgtgattgaagaacgcgtggttagaaacaaaaaagcagaaaacagtacgggtgaggctaaagatttcgtggatgttttgctggaaattcagaatggaaacgaaactggctttccgcttcaaagggattcattgaaagctattctcttggtaatttagcatctctcttactattacttattcactttttggcatatcttgttttaattttttgttttaagcacttagatataattcgtgctaatgttttgattcatagttcaaatttcaaaactcgggacttaaaatttcataatgggaaacttaagtaaccgtacactttggcaatctagtttaccaacacggctaaagtatgtacttaattgtgtgtctttcatttaatgcaaaggactcgtttattgctggtgtggattcaatatatacaactttagagtggataatgatagagcttttgaggaatccaagagccatggagaaattacagaatgaagtgcgaggattagtccaagggaaagcagagataacagaggacgacttaggaaatatgttgtatctcaaagcagtaatcaaagagagtctcaggcttaatccaccgtttccaataccagttcctcgagaatcaatggaagacgtaaaattactagactataacatacctgctaaaactcaagtccttgttaatatttgggcaatcggaagagacccattatcatgggatgaaccagaggagtaccggcctgaaagattcttgaatagtgatattgatttcagaggactaaactttgagttgattccgttcggagctggcaggaggggttgcccaggaattccttttgctattgtgataattgagctaacattagcaaggcttgtgaacaaatttaattttgcattaccacaaggaataaaaaaagaggatttggatatgagtgaatgcactggcatctccattcgcaggaaattgcctttactagcagtggcaactccgtgcagtgtttagtttttttcctacaatttcttataatgacataaaatataaataagtatatatgtaataagtagtaaactacttttagttagtattttgaaacccattgaaaagggcagtgcgagagaatttcagaattcacttatgtaataagtttactggttggctccttttccctatggtgtttattatatctcttttatattctcgatcgcaagatatgttaatccggggaaacaatactgagccaagaaaaaaataacagataatctgttacggggtgatcgtttccttctgtttatgtggcgaggcttctggctatatattacatttgtcctcaattcccaatttctccatatttctacataaaaatggagcttccttggtattctgttgttgttcctctgtttgttttcattttctttcttcataactgctttactacttccaataataataaaaaaaatataccacca

SEQ ID NO: 7 - DNA, 1934 bp, Solanum lycopersicum
  source 1..1934: mol_type = genomic DNA
gtaggcgtggattttaaaagactactttgacattgacatgtttgtgggtacatgctaagatatttaggagtgattgggtaataattttattctaatcctatcttgcttaccagacgtgtttggtataatatagaggacatgttatacaccacaccctctaatcttaccagacgtgtttggtttaatattgaaagtattttctcttcttattcaatgactttgatatggacaattctagtggttggtctatctgtatatgccttgtatgagctgctaaacattcacaagagaaaaaggtatcctccaggtccaacagggcttcccattttaggacatcttcatttgttaggcaaaaatccacacaaagatttacaaaatctagcccaaaaacatggtcctattatgtatatgagactagggctagtacctacaatcattgcctcatctgctgatgcagccgagaaggtcctcaagacatatgatcacatctttgctagtaggcctcatcatgaggcatctcaacatttggcttatggccaaaagaatttggtttttgcaaagtatgatgtgtattggcgcaacataagaaaattgtgcacagtgcaccttttgagcaatcaaaagatccattcatttcaatccatgagaaaacaagaggttgaacttcttattgagtcgcttaaacaggaagctcgtgatcgcgtagttattgatcttagtgcaaaggttacgtccttgaatgctaacttgacttgcttaatggtgtttggaaagaagtacatggatgaggacttggacaagaggggattcaaagctattgttcaagatgtcgtgcatttagcaggtatgccaaatcttggtgattttttccccttccttggtgctattgatctccaaggtatcactcgcaagctcaaggatctttcaaaggtgtttgatgagttcctggagaagattattgatgaacatgtttacgctcatgaacataagcaaaacaaggactttgtggacaccatgatggacataatgcaatctggagatgcaaaatttcagtttgaccgtcatcacataaaagctatcctttttgtatgtactttcatttcattcttatacacaccacaagccttgagaatgaaatggtttatattaattgattcccctgtttcctatttactatacaggacatgcttatagctgcaatggacactacagcatcatcaacagaatggatactcaccgaacttcttaggcacccccaagtgatgaaggaactccaaaaggagttgcaagaagtggtaggcctcgatagaatggtggaagaatctgacctggagaatttaaagtacttagacatggttgtaaaagagggcctgaggctgcatcctgtagcgccactattttatcacgagtccatagaagattgtgtagtcgatggtttccacatacaaaagggaaccagaatcataataaattgttacgcgattcatatggatccaaatgtttggcctgatcctgagaagtttttgcctgagagatttgttgggagcagtgtagattttcgtggacgggacttccaacttgtaccatttggctctgggagaagaagctgccctggaatgcagttggcagttatggtcgtccgccttgtggtggcacaattggttcattgctttgagtgggagcttccaaatggtattgagtcttgtgatttagatattgatgagaagtttgggatagtaacatgtagagaaaaacctttgctggctattcctacatataggctaaacaaatgatgcagatacaatgcatatagtaaaagatcaactagtatttctcaactttacattttgttacaatcaacattggaagtttgtagtatatgattttgtgattgttgtgtgctaatctacaatttacctcattgatctcttctt

SEQ ID NO: 8 - DNA, 3573 bp, Solanum lycopersicum
  source 1..3573: mol_type = genomic DNA
gaggaaaagaaaagaaactcacataaatttggacggatggagtattatattcaatggggtcaaatttgtcttataccattcatcttgtacaaattaatggacaacaaaaaggaattattttttaatactaagctgttcacagaacattcttgagatcatcagtagaatatcacatataaactcatcaaattttgcagatttaacacaatcaagaatggacttaatcatttttattgtaatgttgttagtgatggttttagtagtagttttattagttctattaagaacaaataaggcttctccaaaagaaatggaagcaattccaggtaccttaggatggcctattattggagaaagtctctctttcatatccgaattttcgagccctgctggtatatacagcttcatcaacaagagacagaaactgtgagtacatcataatatagcacgtcgaaaattgttcaattttttctccagggacaatcctgaatatgtgctatgttttagaaaggttataaagttgttcagcaagataattcttgctatatataagctttggagattgatctaacattgtcattcccttcagaaaactcttcaacatgctatctttacatcatttcgcgaagaaaactctgttatctaaagtctgacctcaattttatgatttgtgttatggttttgcttattaggtatgggaaggtgtttaagagttatgtgttgggaaggtatacagtattcatgacgggaagagaagcaagcaagatcttgttgacaggtaaagatgggatagtgagcttaaaccttttctacacaggacagcaagtacttggacctacaagcttgctccaacagacaggagaggcgcacaagagactccgaagactaattgctgaaccactttctgttgatggtttaaagaagtattttcagtttatcaacagtttagctattgaaacattggatcagtggtctggaagaaaaattttggttcttgaagaaacttccacagttgagtcttgtgatcattttttcctagagtttggtctaacggaggcatttttgaacctttcgcatgatgttttttgcagtttacactgaaggtgataggcaacatgataatgagtttagagccaactggtgaggagcaagagaaatttcgaaccaatttcaagattatttctggctcttttgcttctttaccctttaaagtcccgggaactgccttttatcgtggcattcaggtaacaaattgttgatattctatgcaaaatatgatgaacggacagcttaaaagagatgactgtctttaaactacaggctcgtgatcgaatgtatgctatgttagactcgataattgaccaacggaggagtggggaaaacataaaacaagatttcttgcaatccctggtgaagaagcatggcaaagatgcacccgaaggagatgatgatgacaaactcacagataaacaacttaaggataacatattgacgcttctagttgcaggccacgatactacaactgccgctttaacatggctcctcaaatttcttcaagaaaatcctgctgtattggaacgactaagagtaattcttattgaacctaacagagaagagcaacctactgctgacattagtttctaacttagaatcttttattacaggaggagcatagagaaatccaagctcaaaaacaaggcacgttagatctcacctggtctgaagtcaacaatatgccttacaccgccaaagtgagcttgcatactagtcataaatccagcttctgttcatccctgaaatttaaaaaaatttaactttcttttgtgtatatttcaatgttcaggtaatcagtgaaacacttcgaatggccacaatccttccatggttttcaagaaaagcagcacaagattttgagattgagggtaactgtcagatttattgatatctagattttgtatgatgtaaaatgattctttaaaaccatgttcagggtgcaaaatcaagaagggatggtcacttaacttggatgtagtgtctattcatcgcgaccccaaaatatttcctaatccggagaagtttgatccttctagatttgatgtaagttcttcaacataataagcaggctccttctattcaacacctctgagattttcataacaaagaaaattgttgaattctgtacaggatcctctaaaacctttcagcttccttggatttggaagcggaccacgcatgtgtcctggaatcaacttagccaaactggaaatttctgtctttctccatcatcttgtctgcagatataagtaaggaaaataaacaagaaacagtattcttgaaagaaaaaaaaatctaatttcctgtgggcaactgcattacttacttgaaaatttgtcttccttgtagatggacaccgttagacacagatgactctgtccagccaacacttgttaggatgctgaagaacaagtatcccgtcatggttgagccactttagataatcttttacattgtactacaaaaagaagaactagaccattttcagatatataggataatgtaaggaaaaataaccatggtgctgtagttgttcctaaagatccagtgttaaattgaaatatataaagtatggaactctttggtaatgattaaatatttgcagtaatgatatatctttacgttcaaagcaactacttgctgaaacaaatattgcactcataaccgaacaagtgcagaggaacacacaaaaaattcacaaagttgccacgtaattttggcaaattcttccttatagaaaacacacatacgcagaagcataagggccacaataatatgcttctacgagattcgaagtcaaaattcaaacatcacttctacaatgaatctaaagtttcatacagaaacaaagagaaaccaattatagcttctctaatacaaacatcgtttttacgatgcctctaatgtttccgacagaaacaacaggaaacctatagcttttctcttctctatctaagagatcacttagtgctgagggcagcaacagtttttactcccaatatctgaagcgagacaaggaggtttgacgtcctcatccttgtaccagattgatcgtgcttcaactaattcgtccaagaagttatccagcttatcaactgtcacactaggcatcacaacaacatgtgccatatttctttcacaagcaagttgccacttgcgaacaaactcctcatctttaggtcgctcaaaaacaactgtgctgctcagctcattcagcatagcactgattccagctcctataaggcggtctttcaaataatgagcattcctcaggcacttctggacttccttctggaatcccttgtacccttttctgttcagagtgtaccaaaggaaaagtggagcatgcccattccgacttcccatgatagtggcatcccttgatgcaaggtactccacattactagaaagggcattaatgtgctctagccttgttagttgaacaccacatggcattggacatcccacaaacttgtgaccagagacactcacactaccgattggctttttgaaagtaac

SEQ ID NO: 9 - DNA, 2955 bp, Solanum lycopersicum
  source 1..2955: mol_type = genomic DNA
tgagtggatccaaacagattggacaatgatgtttttgtcttcgatattatagagagaaaagctaggccttatcttgtccaaataacaacaaattaagaaaaggtaaaattagctattttcccctcttttttctatgtaacaataaacagtttgaaaaataaaatttcatggagattttgtataacacaattatagcaacaatttgtgttgcaatattgttggtatatacatggagagtgttgaattgggcatggtttaggccaaaaaaattggagaatttcttaaggcaaagaggtctaaaaggaaatccatataagttactctatggagatttgaatgaactcacaaaaagtatagttgaagctaagtctaagtcaatcaatatctctgatgatataactcaaaggcttatcccttttttccttgactccatcaacaaaaatggtatgtttattttattgattcgactagaaagtaaacactgcaactttgagtatgcatatctaaacaccttaactttatgtgattcgtttatctaaactttatgtgtcacgtacgagacacggtggagacaagttggaagtgtttagttgctagttggaccaagtttaggagtctagatttgcacctcaaagttggagtgtttacttgtcagctgatatcatatttgagtgtttgtttatgtagtatatgcctttttttataattgaaaatggatttttggcaggtaaaagttctttcatgtggctaggcccatatccaacagtgttgatcacaaatcctgaacatgtaaaagagattttgacaaagaattatgtgtacctaaagcaaactcatccaaatccatttgcaaagttattggctcaaggtcttgtgttggttgaggaagacaaatgggcaaaacacagaaaaataatcaatcctgctttccatgttgagaagttaaaggttaaatatacttcttttttgttgtattgaattgggatatgacacaagtatcccctagattatgatcgaaatctcacagatacaccttaactaatctaaggtcctattactccataaactcattttttgtgtaattttgtacaacttttggcttacgtgacacactctgtgactccacttaagtgaggcgcgtgggagatgtttggaggtcacgtaggccaaaaaaggtgtacaaaattacaaaaaaaaaagttcaagggtaatagaacctcagtttagttaaggtgcgtctctaaaatttcggtcatagactatggttactagtctcttatccctattgaatttaggccaaaaaaagcagaaaattcaattttcttttttttttcatttgttttgtacagcatatgttgccagcattttatatgagttgtagtgaaatgataagcaaatgggaggatattgtttcaaaggaaacatcatacgagctcgatgtatggccagaccttcaaataatgaccagtgaagttatttctcgaaccgcatttggtagtagctatgaagaagggagaatagtgtttgaacttcagcaagaacaagctgagcatataatggacataagtcgttcaatatatataccaggatcgaggtatgataatttacttttgagatttctacataatacggaaactaaaatgttcattacttaaatttgttgtgttaggttcttgcctactaaaaggaacaaaagaatgctcgaaatcgaaaagcaaattcaaacaacgattaggcatatcatcgacaaaagattgagggcaatggaagcaggggagactagtaaagatgatttattaggcatattacttgaatccaatatgaaagaaattgaacaacacggaaacaaagatttcggattgacaacaacagaagtgattgaagaatgcaagttattctattttgctggacaggagaccacttcagtgttgctcgtgtggacgatggttttgctatgcctacatccagagtggcaagtacgggccagagaggaggttttacaggtcttcggaaatgaaaaaccagatttggaaggactaagtcacctcaaaattgtaagtactttccgctatgtcgttggcatagatcaaacttcaacaatcaaggaaactataaaatcaggataaaacaagtagctgttaatgtgaaatcgttccgactgggctcagatagctaggccacttctataatcctatttcagtctcggaagggcaattactatgagactaaaatatctcgcgaaggcatgacccctgaattttgtaagaagtgaactaattctacttccaacttacgcaatgtattttttttccataggtgacaatgatcttatacgagacattaaggctattccccccattaccggtatttagtagaaggaacaaagaagaagtcaaattaggggagctgcagctaccagctgaagtgatacttattatacctgcaatctttattcattatgacaaggaaatatggggcgaagatgcgaaggaattcaaaccagaaagattcagtgaaggagtgtcaaaggcaacaaaaggtcaagtctcgtttattccgtttggttggggacctcgaatttgcatcggacaaaactttgcaatgatggaagcaaaaatggcaatagcaatgatactacaaaagttctcctttgaactctctccgtcttatacgcatgctccatttgcaacaattactattcatccacagtatggtgctcctctgcttctgcgcaaacattgaaatggatattcttcatttatcagctcgaatactactaaatagttgagctatgactgaagtcacccttttcgaatttcaatgttgtagttttgaaatgttatcctcgtgtcttttgtgagcgaataagtttatgtaatgaaaatctatataaactgctgcatataaattgttcattcccagtcctatttatacataatttttttttttaacgat

SEQ ID NO: 10 - DNA, 4204 bp, Solanum lycopersicum
  source 1..4204: mol_type = genomic DNA
aaaatcagatccactcatttatattattttattaaaattttgttgctataattgtcaacttgcgtagtagaacagaaaacttttgttcgtcactcatgacgacatcgtttttggagttcacatgaacatgccaggtgtttttaagtgcaagaaactatagtagtaaatttaacagagaaaaaaaaaacttgacaaaattgaaatgatagtaacaatattttgttgtgccatagcaattactttgtttgtgtgtctatggaaagtactaaattgggtttggttccaaccaaagaagttggagaaattgttgaggaaacaagggctaaatgggaattcatacagatttttgtatggggacatgaaagatttttctaagatgattaaggaagctaattcaaagcctatgaatatggatgatcatgatttagcaccaagaatggtgcctttctttcttgaaaccatcaagaaatatggtaagttttcactcaaaaagtttattcattcgtaatcgagtcaaaaattaaagtttatgaatttaatctttttttagttattgcataacttataaagttgatgtcatctaatcagaaaatcacgagcgggttcgtagacccaactttatgtagacatgtaggaattactacagatcattggaaacactttatgcaatagactctcgtagtccgttcctttccacacctgctataagctttagtggatcgaactgttttatactaaatactttatttgttccattttatatattatctttttaccaattttacatattatctttttactttgcataatgcaaaaagaaataacgtaactttacctttctattcttatttaatttttttaaactcaagttttcaatcaatgaatatgaattaatttaaccaatagacataaatcatttacttagcttttctaagttggttaaacgcatattttcaaagctaatagctcacaaggtaaaagaggaacaatatactctctccgtccagtattatttgtcatggtttttatttttagaattaaactattaaaactttgattaattttttaagatgtattttttcatcatattaatatgcaaaaaattgtaatttattgtacttttcatattgttttagaatatctaattttttttgtttaaaatatcgaattaatgtggtctaatttacctttgaaaattaatcaaattgactttcgataagcgcaacatgacaaataattccggacggagggagtaatatttatgaattgattttatgaaatgacaagtactatggatgaactatttatagaaatgaatgagatacaaaatgatacagaaggagtactaattattgatataagtagtagattaaattcactaatttgttatccattcacaaatctctcattcatttgcacagacggagttagaattcgaagtttaagctttttgaacttgaagttgtagttctctttagttacaaggttcacaattaaatatctgtacatatcttatgaacttgctaatacaaataagcagtttagctagaagttcctttggtaaaatgtagtttgtttgaactattttttcgaattcgataaaatttgtaatgagcatgttatcctgatttctttatcttttaaagatcactctgttcatttcgttctgttcgattgtatagatgttttgtttcgaattttgtacttgttgtagttaagcttgagtcatatatatatatatttgcagggaaaaaatgttttacatggatgggtccaagaccacaaatactaatcatggatcctgagcttataaaggaagtactttcgaaaacctacgtgtatcaaaagcctcatggaaatccgttaggcacgttatttgtacaaggattagtaagctatgagaaagataaatgggccaaacatagaaaaatcatcaatcccgcgttccatctagagaagttaaaggtttgtcattcaacttcgtgatttgccttccttatcgttcaatcttaacttttatgtcatatgactatttcgagtaatgtggtatctgctgcctgacttgatgtagacaatagtggttctatagggacgcgaatctcacctggatcaatgattcaatgttctagattgttcttccaccaatcgataatatgttcactcttgcttcttgtatgttccacatatggctatattgagtgatttctgctataccttagcttgagttagacaatccgagagtgaatctatggttcaaagaagtcgaatattgtctggatcattatttgtcaggacgctctttgcgtaagtttcttggcttggacagagtacgtgtttagtccatgttgtgttcctacagactaagttcaattttatttgatatatttactatatggtgattgaatatgtgttactaattatgttcaaagtgcaagtgttattgacgttttccctgattcactattttctcgaacagcatatgcttccagctttttacttgagctgtagtgagatgctgagcaaatgggaagacgttgttcgagttgaaggctctcatgagatagatgtatggcctcacctgcagcaattgacttgtgacgtgatctctcggacagcttttggcagtagttatgaagaaggtagaaagatttttgaacttcaaaaggaacaagctcagcattttctcgaagttatacgctcagtttatatcccaggatggaggtaaacttattgctccaaagaatccgaaaaatgtagctataggaaatgattagttctaatatttgttaatacgttttggttttgtgttttcaaagatttttaccaacaaagaggaacagaagaatgaaggaattaaaaaatgatgttcggtcttcaattagaggtattattgataaaagattgaaggcaatggaagcagggaacgcggataatgaggatctattgggcatattgctggaatcgaattttaaagaaattgaactgcacggtaacaaggactttggaatgagtatcgaagaagtcattgaagaatgcaagctgttctattttgctggtgcagaaactacatcagtgtggattctgtggacgctggtgttgttgagcagacatccagattggcaggaacgggccagagaagaagtattgcaagtgcttgggagtcgaaaaccagattttgatggattaaatcatctaaaagttgtaagtattcttgttctgccattggtacaaaaagcacgtgtgaatttcttttgatcatctcgttcatgctagaggagcatctctgttacaggtaacaatgatcttgtacgagtcgttgaggttatattccccaataacagtacttacgcgacgagtttatgaagacattacactaggagaagtatctctaccaactggtgtgctagtctcattgccaatgattttattgcatcatgataaagatatatggggtgaagatgcaaccaagttcaatccagagagatttagtgaaggaatatcaagtgcaacaaaaggtcaagttacatattttccatttgcttggggtccaagaatatgcattggacaaaattttgccatattagaagcaaagatgactctgtgtatgatcctacaaagcttctcttttgaattgtctccatcctacacacatgctcctcaatccttggtaaccactcagcctcagtatggtgctccacttattttccacaaactataatttcacaatctcgtgaagagtttattcagactcgattcagtgatggagttacaatatcgaaatacgggttcagaaacagtttctctatttcataaagacaggggattatgtgttgtactctccactctcccctgaccctcatattgggtatgttgtttttgttattcggacaaactcagtagttttcgttaaaagcttgcatttgtttgagcaaatccactaaaaatactatacaacatccgtgctagcacgaactcaatatatattgctttatgtgaaatagttgaaatttggagaattacctctattttttttaatatggtttttaaatattttaggttgtctatattattatgtttactgttacaatattgaaatttttttaaaatattcacttaatgtagacatgtgttacatacgatgatttttttcggtgccaagtttatgagggtagggatacaatctatgtattttaggattataatt

SEQ ID NO: 11 - DNA, 7931 bp, Solanum lycopersicum
  source 1..7931: mol_type = genomic DNA
aaattcaactcaaaacaaaatggacgaaattcaaatattagtaagagtgtgttgttctgccatagcaattgctctgtttgtgtgtctgtggaaagtactaaattgggtttggctcaatccgaagaagttggagaatttgttgaggaaacaagggctaaatgggaactcttacaaaatattgtatggggatttgaatgattttattggaatgattagggaagctagttcaaaacctatgaatttgtctgatgatatagctccaagattggtgcctttctttcttgagaccaccaagaaatatggtactcctgtatactttgcttattactctaatttctctagaccgcactcgtagagttacaccgtattttaaatttttcggtcaatcataatttgatacacgagtaggtagaagttgttatagtaggtcaaagtctatttatatggacttgaacaattctcctctcatgagctagctttaggttaagttaagcccaatgttatatctttgcatgttgtagtattagagtcaggttcatccccgtttgagcatcacacaacactctagttgggcctaggcatgataggggtgttaggataggtcaaagtgctatattggttagggattagactagtggtctgcttatatggctatggcaattcctttctcatgagctagcttttggttgagttaaggctagttgtagtatcagagtcgggttcatccccgtttgagcatcggtctacactatagttaggcctaggcgtgatagggatgcaggggtgcacccacagaggttcaagtgggttcataaataacaccatatatatatatatatatatgatcagtttttaaaattttatatctatatattaacttttgactccactaacacaagtgtgacccatggcacagtggtgaaaagggcttaatttgcccagcctgcccgagtttgaatccctatttcccacattttagatttaggataggtcaaagtgtcatatcggttggggattagactggtggtttgcttatatggctatgagctttgataatccttttctcatgagctaacttttacagttgagttagactcaggtgtcgtatctttacagaggtcctggtttgagtattaccgccacacacctattatcgcaaggatatacacatgtttggttcttaaagtgaatcaaacaatgattgtgattaatgaagctgatttaattgagaatgatttgtactagtagttaatcctaagacatatgcttgcagggaaaaaatgttttatatggttgggtccaaaaccacaagtaataatcatggaccctgagcttattaaggaagtactctcgaaaacctatttgtatcaaaagcccggtggaaatccatttgcagcattattggtacaaggactagcaacctatgaagaagataaatgggccaaacatagaaaaattatcaatcccgctttccatctagagaagcttaaggttgcttttcttccgcgccctccttcaaactttgtgattggtcaactagaaattattatgtttgtacacgacggataaaatgctggctcatgcttctttagctactgctctctacaagtctttctatcccctttaattcgctatccttatctttctatctaatgttttatgtattctgcatatgactattgcaattaaatcctgatatttaacttgagttagacaagtatagaagggtcttatggttcttcagtgtcaaatctcgtctgcatcagcgttcaatgttctagttttttcatccactaaactataaaaatgctcactcttgcttctctggctacatttgttgtgtaatgtcttagatatattatggatctgtttttattataaagagatttgtaatattcctgaccgtgtttcgccttatttgttatttgaactctgttcttttttatcgaactagatactgacggtatcactatatctgtattagctaattctgttttaagtgttgtctcgttgacatttgtcgaacagcatatgctcccagcttttcacttgagctgtactgaaatgctgagcaaatgggaagatgctgttccacttggcagctctcgtgagatagatgtatggcctcaccttcagcaattaacttgtgatgtaatctctcggacagcttttggaagtagttatgaagaaggtagaaagatttttgaacttcaaacggaacaagctcagaattttatcgacgctgtacgtgaagtttatatcccagggcggaggtaagttaatttatcctaggaattgaaaaacggtagtcatagctatgttgttcaaacccttcaaaaatgatgacagttggaattccgatcctccaaaaatagcgtttttttggaggatccgacatgaatgcagtaacgtttttttggagagtcggaacaacatagagccaaagtggtctattataaactacaaagttcattaggagatgcacatagcttttacttcatctaattaatgcattttggttttctggcttataatataatctcaaagatttttgccaacaaagaggaacagaagaatgaaggaaataaaaaacgaagttcggacctcagttaaaggtattatcgataaaagaatgaaggcaatgaaagcagggaatgccgataataatgaggatctgttaggcatattgcttgaatcaaatttcaaagaaattgaacagcacggtaacaaggattttggaatgagcatcgaagaagtcatcgaagaatgcaagttattctattttgctggccaagaaactacatcagtcttactcgtgtggactctggtgttgttgagtcggtatcaagattggcagacacaggccagagaagaagtcttgcaagtgtttgagagtcggaaaccagattttgatggattaaatcatctaaaagttgtgagtattctgtttttttttgtttgtttttggtttacttcatctaaaagcagaagtagtactttggtagaggtgattagacaaggcataacgcaacttcagtttaccgaggacctgactttagtactctagtgaattcatgctaagatatacacctaaaggagaagcaattttttgcaggtaacaatgatcttgtacgagtcattacggctatatcccccattgataacacttaatcgacaggttaacgaagacatagtattaggagaattgtgtctaccagctggtgtactagtctcattgccaatgatcttattgcatcatgataaagatatatggggtgaagatgcaaacgagttcaatccagagagatttagagaaggaatatcaagtgcaacaaaaggtaaagttacatattttccattttcatggggtccaagaatatgcattggacaaaattttgccatgttagaagcaaagatggctttgtgtatgatcctacaaagcttctcttttgaattgtctccatcttacacacatgctcctaaatccttagtaaccatgcagcctcagtatggtgctccacttattttgcacaaactttagttttgtaatgtaatgtaacaagaattaatataaagactcaaataaggggtggagtttgcaatattaaatatggatcgtttgtgagagatgtcttgtaatataggtaagagttaaaagggatgaagttgcaattttttagggctcgtttaaatatgtgatatgaaattatgattttaatttgtttagacgtaatatgaatcatcacttatacattaataatctgcctgtgattttaatatttttcttaattagttcttatcttagtctaattatttatttgtcatgtattttttttaaattttacaaacggtagctaatatttttatctttgtattttgcttttattctttaaataaatagacaaagggacaaaaaagaagaagaaaaactaacaaaaagggaaaaaggaaattaagaggaaaaaaagatttaacctttttagaaaaaaaaaaagactgagccaactaaaaggaaatgaaaatttaagataaacaaatttaaatgtttttgcattattttgtttattttaaaggttttaagtttaagagtactcaattcccttgcttttagctaatattcgttatgtctcaaattatgtgatatttttaaattttaatgataaaatttatctttaacatgtaattttgttaatatgttttaactaatttcaatagtcaattattataatttagaataatatttttttaatataattttcaaaaatctttaaaatcctatatttaaatttatgattaacttaaaatagttaactatcaaaatttagatgatataatataaattgactaattgagacaaagaagtattagacgtaaacacttcttgatatttggtaacatgatcaaataagtcatcaattattgcaaatagttcatttaacttatcatgttaaatatctcttggcaatgcttttttaaaaaaattatatggttcatgtcttttttaatatttttctttagttatcttcgattattatttatattttgataatcttttaacgtggaaaaagtttatggacaaaatatttccaactaaagtatgaaaaactccagcaaatgatattgaaattcaaagaatttcgggtttataattacagatcgtgtagattatcttttcttaatagcatatagaaatataaatcgtgatagaagagattagcttaagaaaagaaactaagtgtattttcctcttaaatatttgagtctcgatccgctttcactccttcgaggcaaagtatattagaaaaaataatacaaatacgattgaagaagaaaatgtctttgagatatttaattactttctttaaggagatattgtttgtatatatatattgagaaaaattcaaacgaatccttaatttattcatgcatatttttcttagggtaattataaaaaatctttatatatatagaatttaattaagagatatcttttcatcagaacgatattttttcatgaacatatgacaactttttataacgattatgttctttcatgaacatcaatttaagaactctatatattacaaagagaaaagacataaaattaccgttgaagttgttttgaattttcaaaaagacactttaactttgcgaatgtcctattaccccacgaaactatctaaatcttcaataaatatatcattttgactaattttaacatcactcgtgttgtcacatcatatgggcgcgtgaagcactgttgatacaacttcataccaatgtaaaattgttatttgacacttccaatttgccttattgaaaaaaatataaaaactcatgtattaattattttttttaattcattaagaataatttttaatttttcaattttttttctcatcttccctaccatcattagcttacccatgtttcttgaatatccatatcttccttcatttctttttctctttttcctttctggcgttttttttaaattaatgttattgaggggtttttttcgaaaaataaattagtttggtgatttttttttctcttataaattaagattgggatttgggagtagttaaactatgaaaaattcaacttcgaagtaagagttagaaaaggtggtgatttaaatgtcagatttcacttggtaatatttgagtatttagattttaattatttgacgatatttatttgcataaataaattaattgatagtaatttgactaaaaattgatagtgctgctgtgaaatctgctcaaccacaatgaatttgaagattcttgaagcacaaagagaatggaaaaggtgaagagaaagagaaaaaacgaagttgaggtgaagaagaaagagaaaaaggaaaaaagaatataaagaaattacgtacataaaaaattaaatattatttttaatacgtggcaagtgtctattagtgcgtgacatacaaatatatttgtataagtggtattgagtgaaaaatgaggtatacattctactttgaagttgtttaatgaggtaataggacacctgcaaagttaaaatatctttttaaaaattgaaaacaacttcgatgatatttttatgtcttttctctgtaacaaaatatatatacatgcttcctaatttggtgttttatgactattgcacactcgaccttccacgtgtttgttgcttacttgaacacgtatcatccatctatttttgtcctatacattaatataaaattattcaaaatagagacacgtcatctataaattattgttggagttatgtcactttgtgctttaattagacgtgtttttctatattgattttcttaatgatttggcaatgttatgctcagaaaagaaaccaataatgaatatggggtatatagaattgtcattaaacgaatatcataatataaagaagtaaataaacataagagaaaattaaatgagattttactatctatacataaaaaataagtttaatatgaatgaataatgcaatttctcattgaacggataatttaatctttaattctacatagctcctccccctccctaaagcaacaattaagatgaattatgatgaaatgatagagatctcgacatttaaagatttcaaatttaaatttcaaaaaatgattatgataattaagataatttgagatatcgataatattccatgtagagacggctctacggaataacgtaaaggataagttatgagttcatttagatttaatatttcaattcagattaaaattatgaataagaatttaattgaataagtgcaagtatttagggtgggtttactatttagttagaaaatttcttccttacacttttcatattgatatattcacatttttgaattgttcatcaattcatgtttgattattctcttgagtaaatgcgtgaaactagtaaaatttatatttttaatcgaataagtgcaagtatttagagtgggcttattatttagttagaaaatttcttcctcacacatctcatatcgatatattcacatttttgatacgttcatcaatttatgtttgactattcttttgagtaaatgcgtgaaaccagtaaaatttataatcgattcatatatttaatccatcatttacttttatttatctattatattaaaaatgaattatttaataccttagtgttttacaaatttcaaattattttccaaattaattaaagattatagactaactaatacgtgtataatgaattattaaatatgcacgtcatcaatttttcttcattaccttataattatttatttattttttaaattattttttaaaattttatcttaaatattaatttattatgataatatatgtacgtccatttttttttaaagaatcattcaaagtttaaatcagataactaagattgaatagaaaatatttatcgagaactaaaaatacgcgggtgaataaataaaaatgaaagaaataaatttaatataagccatctatatatataatacataccatgaaatgacaaagtgacaaagtagagtgtaagagcttattatatagtgaacaattctctcttttagatcacaaatcccttgaacaaacaaagcaaaaaccaaatcatttttctaagttaaaaaaaaaaaatctgatttttgtttttcttactggaaaaaagcttcttttttttcctttcttcatcatctgggttctaaaataaatcaagattcaggtaccccattatttataatactctttttttcattgccatacgctcaaaatttgatctttttttctgtttcatgtggaccctttaaaatccctttttttaaaaaatattctgttttgatggggtaatttgtattaaaaaattatgatgatctttctgttgtgtcccactattttatttatttttttaaaataattttttgcaggattttga

SEQ ID NO: 12 - DNA, 1509 bp, Solanum lycopersicum
  source 1..1509: mol_type = genomic DNA
atgatgataatagacttagagttttctactctactcatttgccttgttccattcctttttatcttcttcatcaatcttttccattccaaatcaaccaaaatcccaaaatcttatccaatcattggttcatatttctcaattttagcaaacaaacatcgtcggattcaatggagttctgatataattcaaacaactaccaatcttactttcactctcattcgtcctttcggtaatcgccaaatctttactgcaaatccatcgaatgttcaatacatgctcaaaactcattttcatatttatcaaaaaggtcatatgtttaaatcaaccatggctgattttcttggtgatggtatttttaatgtggatggtgaaatctggaagtaccaacgacaagttgctagtcatgaattcaatactaaatctctacgaaaattcatcgaaactgtcgtagatgctgaacttaacgaacgtctgattccacttcttgctgttgctgcttctgagaaaaaagttgttgatctccaagacatattacagaggtttgctttcgataacatttgcaaaattgcgtttggatatgatcctgcttatctatcgccgtctctaccacaagagaaattcgcagttgcttttgaagaagctgttaaattaagcagtgaaagattcaatgctattttcccctttttatggaagattaaaagacatttcgatttcggaagtgaaaagagattcagaatcgctgtagatgaagttcgtcaatttgcgaaacagctcgttagagaaaaacagagtgaactaaatcaaaaatcatctctcgattcagtagatctattatccagattcttaagcgttggtcatagagacgacgatttcgttacagatgtagttattagtttcatattagcaggtcgtgacacaacatcagctgcattaacttggttcttctggttaatctcggaacaccctgtggtggaaagttcaattctaaacgaaatcaaattaaaatcccacacgccagtatacgaagaagtgaaagagatgatatacacacatgcttcactttgcgaaagcatgaggttttatccaccagtaccaattgactcgaaaacagctacagaggacaatgttttaccagatggaacttttgttaaaaagggaacaagagtaagttatcatccctacgcaatggggagagcagaggaactatggggaagtgattggaacaagtttaaaccagaacggtggttggataaagatgaagttactggaaattggaattttgttactaaagacatgtatacatatcctgtttttcaggcggggccgaggatttgtttggggaaagaaatggcgtttttgcagatgaagagggtggttgctggtgttttacggcggttcaaggtggttccggtcacggaaaaaggggtggaaccggtgtttatttcatacctcacggcgaaaatgaaaggtggtttccatgtgacaattgaggaaagggaccatgagtag

SEQ ID NO: 13 - DNA, 3852 bp, Solanum lycopersicum
  source 1..3852: mol_type = genomic DNA
attccttcatctcccatttgtttttctataattctatcctttccatcaattttatctttatccacccccaaaaaaatcatctttttttccccaaaatatgaatcatcttgatgttttttggtggctgaagtcttttcatgtctattctattcttatcttcatcataatttctctattttctctaatattttacatggtgagggtaaaactttggtgcaattgtgaaatttgccatgcttacatcacaacaagttggtcttcacaattcatcaatttgtgtgattggtatacacatcttttaaaagaatcaccaacaagaacaatacacatacatgttcttggcaacatcataacttcaaatactcaaaatgttgagtatatgctcaaaacaagatttgataattatccaaaagggaaaactttctctactatcttgggtgattttcttggtagaggaatatttaatgttgatggggatttatggagatttcagaaaaggatgtcaagtcttgagcttggaaaagtttctataagatcctatgcatttgaagtggtgaaaaatgagattgacaaaaggcttgttccacttttacatgaatataaacaaggtggcggggttcttgattttcaagatgtttttagaagattttcatttgattgtatttgtagattctcttttggattagaccctaaatgtttagaatcctcactccctatgtcacaatttgctctttcctttgacctagcatcaaaattatcagctaaaagagctatgacaacatcacctatagtttggaaaatcaagagatttttcaacataggaagtgaaaaagagttgagggaagcaataaaagtgatcaatattctagcacaagaagttataaggcaaaagagaaaattagctttttcaaatcatagggatcttctttcaaggttcatgggatcaataagtgatgaaacttatttgagagatattgtcataagctttctactagccggtagggacactatagcatccgcgttaacaagtttcttttatgtgattggaaacaatccacaagtcgctaaagccattagagaagaagctaatcgagttctaggtccaaatgaagatctcacaagttatgaacaaatgagtgagcttcattatctacaagcatgtgtttatgaaagtatgagactttaccctcctatacaatttgattcaaagttttgtttagaggatgattttttacttgatgggacttttgtgaaaaagggaacaagggttacttatcatccttatgctatgggaagaatggaagaattatggggttgtgatgctttggagtttaagcctcaaagatggttgaaagatggggtttttgtccaagaaaatccattcaagtatccagtatttcaagctggacttagggtgtgtttgggtaaagaaatggcacttgttgaggttaaaagtgtggctctctcattactaaggagatttcacgtcgagttagctcaaccatatcaccacaaccctcgattctctcctggccttactgcctctttcaaaggtggcttattggtttcagtgcgacaaataagttcataagatcaaaaaattattccatatgtatatatatatatattgttcaggatcaagctgaagtagtaacttataactatatatatgtggtatagcatattcaacaaaccctaatcatggaggatggaactcatttcaacatatcaattaagaagtttttccctctttgagccaaggtaaattttttgttatgttatggcatattaaaaagtatttgcttttttacaaattatatatgttttagctatgaagaagatggtatatgtattttgtttcttgatatgttgaagttgtatatatatatatacgtattgcttgttgggctattgtaaatattgtttgttattattattattattgtgttattattattattattgtaattatgattattatttggtaatgatttactactctatgttctacttcctcaatttgtgcacagtgtgaaagtcgtttagggatcgtttggttcaaaataagaataatttcgaattataattctatgattaataatcattattataacccaaataatcaatctcatattaaatatatgtgattagttataccaaaattttggtataaaactaataatattagtttaaattaatatgtccaaccgaacatggaataaaatcaaatttaaaattttgtgatcattagttctacctaaaagttcaagctcaaaatattaaacgtgatcatctttgtgtgttattttgcatgtgttaaataccaattttattaaaactcctcacatcgaaagttatgactatactaagcagaaaggagaattttacccaaagatcataaataaagaggaaaattaagttggaaacgttaccttcggaatggattttatgatgataatttaatttaatcagaatctaaatatagacatcgaacattaaatgttaaacgaaaaaaaagatgaaagattaaaattttatcttattaagttctgcatctattccgtatacatatttaataaatttatttatcctacttatattgacacgtgtcatgatccaaaaaaagctatttctctgttgtgataaagcctagataattaatacacgttgatatagctaaaagggcaaaaacaacgatttttttttttaaattttatctaattaacattcctagcagggcgccgtataaaccaattttatcaatttcataagtttttaaaacatagaatttaatttcataggtagatataatctaggtaaaaagtgtatctctctataaagttaatttataatttttttaaaataatgagtctattatatatttacaaaattatgatttcaatatattattaattttacactttaaagtcaagatgaatctaatatctccatctgcattgatcattgtccttatccctagtttttctttttacaatagttgcataaattggttggctgagtggttttccgcatgttttattattttattgaatttatgtgattattgttcatcatccttgttagaaaaaggacaagaaattggtgtgctatgatgtatatgacgctatttcgtggattcagcgtttgttaatttttttaaactagtgtcacgtaagttaaaaagggatagaaaattacttattaaataaatttatgatgtaatagtaccttagtatagtataagtatgtctctaaaatttcggatacttgtgtattaaccctaattattttatctaaaacttgagttgatagaatagtcatacttttatgtgcttaattgtatgttttccatatgccccctaattattttatctaaaacttgagttgatagaatagtcatacttttatgtgcttaattgtatgttttccatatgtcccctcttagtgctgagtcttttataagtatcttagtatagtataaatgtatttttgaaatttcgaatacaaattgagagtgtacttgtgtattattcctaattattttatctaaaacttgagtgaatagaatagtcatacttttatgtacttaattgtatgtgtttccatatgcccccttttagtgctgagtctagttttttttcatgagttaaaaatgtgaaaattaattttgattattttatctaaaaatttagcaatagaatgaattgtacttgcatctagctagtctttcgattaacttctctttttctccacgactttcagtacctatttccctttcaatttgttgggtgaagatcacaattgcaatttaagaatcttgaataatcaagttgactgtatgtaattttatcc

SEQ ID NO: 14 - DNA, 2560 bp, Solanum lycopersicum
  source 1..2560: mol_type = genomic DNA
tttcacctccgattattagatatttattaaaatcgatatatgtttagtcatgattttatatataaaatagttatgatcacgaaatatttcgatcttaaactaataaaaccttgatgagttgtttagtgtttgtctgttaaatcaaccacgtgagtgcttacgtgacttctttacgtccaacttgaataacaagttaatctcttatgcaacttcaacgaaagtcttcaaacaattcgacgtatatataacgtgacactaatgcaattacaactcaaaaagtatttactcctctttgtttcattcaagcatttccacaaatgggtcagctacattttttcttctttcccatgatggctcaaggtcatatgatacctacacttgacatggcgaagcttgtcgcttgtcgtggtgttaaagccactataatcacaacacctctcaatgaatctgttttctctaaagctattgagagaaacaagcatttaggtattgaaattgatattcgtttactaaaattcccagctaaggagaatgatttgcctgaagattgtgagcgtcttgatcttgtaccttctgatgacaaactcccaaacttcttaaaagctgcggctatgatgaaagatgaatttgaggagcttattggagaatgtcgccctgattgtcttgtttctgatatgttccttccatggactactgatagtgcagccaaatttagcataccaagaattgtattccatggaactagttactttgcgctttgtgttggcgatagcatcaggcgtaataagcctttcaagaatgtgtcatcggatactgaaacttttgttgtaccggatttgccacatgaaattaggctaactagaacacagttgtctccgtttgagcaatcggatgaagagacgggtatggctcccatgattaaagctgtgagggaatcggatgcgaagagctatggagttatattcaatagcttttatgagcttgaatcagattatgttgaacattacactaaggttgtaggtagaaaaaattgggctattggtccgctttcgctgtgcaatagggatattgaagataaagcggaaagagggaggaaatcatctatcgatgaacacgcgtgcttgaaatggcttgattcgaagaaatcaagttccattgtttatgtttgttttggaagtacagcagatttcactacagcacagatgcaagaacttgctatggggctagaagcctctggacaagatttcatttgggttatcagaacagggaatgaagattggctcccagaaggattcgaggaaagaacaaaagaaaaaggtttaatcataagaggatgggcaccccaagtgctgattcttgatcacgaagctattggagcttttgttactcattgtggatggaactcgacactggaaggaatatcagcaggggtaccaatggtgacatggccagtatttgcggaacagtttttcaatgagaagttggtgactgaggtaatgagaagtggagctggtgttggttctaagcaatggaagagaacagctagtgaaggagtgaaaagagaagcaatagcaaaggcgataaagagagtaatggcgagtgaagaaacagagggattcagaagcagagcaaaagagtacaaagaaatggcaagagaagctattgaagaaggaggatcatcttacaatggatgggctactttgatacaagacataacttcatatcgttaactagtgatgcaaaaaaaagaaaaaacatgtgtgtttctatattctgtcttctgttttgctgatttgatcatattacgtacttcttcatcataattaatgacatcaatagaatccaagatcaatcatctcgaaattcaacgttaaaatatttcgacatttgaataatacatcgaattaaaatggaataattagtactaattgatttattgaatatgtgttgttgaataaatccacaaggccaaagacttttgtgagtgcacgccagatctcaagttcatttaagttgtaattactcgcccagcctaactgttttgactatagttttccaaaattaagtactctgggctctaccattttctgtcattctcgtttgaccatattccctctacaggtgtgatcttttgtaaaatcatgtttattttttcttttcataatgtccatacatatgtaatcagtgttaagaaaaattttcatttaggggttaaaaagaaaatatataagcgtgtaaatcagtcaacaaaatgtgatctttgaattcttttggattcaaaatcacattattaacatgttttattacaaaaaagaaaacgtaaaaacagaatgcataggttcaaaggaacttattaatggactatcttatttttcttgtcattaagggttcaaaatatatatagtcacgtaaatataaaaaatttattttatatatatgatgtaatattttatctaaagattcagatgaatcttggcttacctgtgggtccgccctcctgtgtaaatatctatgtggtttatccagtattgaaataaaaaaagtttatctccattttaaa

SEQ ID NO: 15 - DNA, 2184 bp, Solanum lycopersicum
  source 1..2184: mol_type = genomic DNA
tgtatagtagaatttttttattaacattgatataaagtttaagaaaggaaaacgtctttcttctgtattctagctgaacttctttcattccagagagttctttcatgtcctgaagaacctttatctagtttactatcaaatcctctgtacatttatagcacaaattgttatattaaaccacgcagaccaagtgtttgtgaaaatggctgatgatcagctccatatttttttctttccattgatggcgcacggccacataattcctactttggatatggcgaagttgttcgtttcacatggtgcaaaggcaactattatcaccactcctctgaacttacccatcttcgttcaatccagtgaaaaaatcaaacatttggggctggaaatttatgtcaaagctatcagattcccagcagtggaagctggtttgcctgaaggatgtgaaagaatggatcaattgacttctgatgatttggtacccaaattcttcaaggccaccaccatgcttcaacaacaactagagctattactccatgaatgtcaacccaacgctctcgttgctgacatgttcttcccctgggctacagaatctgctgccaaatttgatatcccaagattggtattattcaatttcatttattgtaacccattgttctctcatatatacttaatgatttaagtttaaattttacaggtatttcatgggatgggcttctttgcgctttgtgctttggagaaccttaggcattacaagcctcagaagaatgtgtgttcggatacagaacctttcgttgtcccgaatcttccccataaggtaaagctcacaaggatgaaattagctccgtatgatcgagaagaaacagaaatgactaagtttattgaacaagtgaagggatccgagttcacaagttttggggttattgtcaatagcttctatgagcttgaaccagattatgctgattactacagaaaggttttgggtaaaagatcatggcacgttggaccctttttgctctgcaacagaaaaaacgaagaaaaaatccacaaaagcgatgataaacatcatgagtgcttgcaatggctggacacgaagaaacaaaattcagtaatctatgtttgtttcgggagcatgtctaactttactgattcccaaatcgaagaaattgcattaggcctcgaagcttcagagcaagaattcatatgggtaattaggaaaggcaagattccagaaggattcgaagagcgaacaaaagaaaaaggattcatcataagaggatgggcaccccaaacgctgattcttgatcacgaggctataggagcctttttaactcactgtggatggaattcgacacttgaaggcgtatcagcaggggttccaatggtgacttggcctatatttgcagagcaattcttgaatgagcaattgataactaaagtactacaaataggagtggcagttggggcagaggaatggagcatagtggtggataatgtgaaaagtgaagcaattacaaaggcagtgaagtgtgtaatggctggggaagaatcaatggaaatgaggaggagagcagaagaagttaagaaaatggcgaacaaggctgttgaagaaggtggatcatcttacagtgatttgaatgcactgtttcaagaactaagaaccattaatttgcacaactaagtgtacttagtcgcactttgagcggtcgtgaaaagaattaaaataaaattacatatcaatatcaataattattttaataaaataaaaatttgttaatcaaataattttatagaagagaaactatgaaaaatatttagatataatatgaaattttttaagaaattaatatatgtgaacttcaaaaaattaagtatgaaaaataaatttcatagagaaaaagggaaggtaataagaattgggtaatattagaactacttaaatgtttttagtataatttatatatatttatgtaaactagaaatctgatcgtttttagtatataatttatatatatttatgtaaactagaaatctaatcgttcttttatgcttatttgtaaatttttttaacttatattcttagtataatttttcgacaatgtgtaactcgggttgctattaataattagtattcataattatataattagtcacactgttataaattattatccacagttactatta

SEQ ID NO: 16 - DNA, 2072 bp, Solanum lycopersicum
  source 1..2072: mol_type = genomic DNA
atcttttaactctatccattttaacatttatttcattccacgtttgtattagggtcttattaaatttaaattcggtcattttatatttagaatcgaatataaaatatctggttaaagatttattttaatactttttttaatttattttgtgtttgtattaggatatcactaaatttaaattcatatgataaaattacacattaagatcacttcatattcagaatcgaacatgagatatctggtttaaggacgaaaaaatacttataaattaacaagttatttgttctttcaatagtcctaacttccttcaatggagaatgaaaaagataaacttcatgttgtgatgtttccatggctagccatgggacatatcatacctttttttcatctatcaaagtgtttagcacgaaggggtcatagagtcaccttcatttcaactccaagaaacattgaaagagtcgtcaataaagttccctctgatcttgctcatctgttgcatattgtgtgttttcatttgcctaaagttgaaaacttgccggatgatgcagagtcatcaatggacataccttaccaaaaagctcagttcttgaagatagctttcgattcgcttgaaatcccattaaccaattatcttgagagttcatcaaatcccaaaccagattggattgtctatgactatgcttctcactggctacctcaaattgcaggtaaattaggcgtttctcgcgccttttttagtctgtttactgctgcaactatgtcattttatggacctccttgtgctttgttaaatgatgaaaggtcaatagctgaggactatacagttgttccaaaatggattcctttcgaaacgaaagttgtgtataggctgcatgagattaagaagaatttcgaagcaccagctgatgaatcaggtacatctgatggagccagatttggtgcttcaattgatgcaagtgatgtcatattgtttcgaaattgtgttgagtttgaatccgaatggttcagcttggtttctgagctttatcagaaacctattatatccattggtgttctacctccatctgttgtagttgatcatgaacaagatgacagcaatgatgcgtcgtggtcaggaatcaagaattggctagataagcacaatcaagattctgttgtttatgttgcacttggaactgaagcaacactgaatcaacaagagctgaatgagctagctttggggttagaaaaatgcggattaccattcgtttgggtattaagagatcagccaaaacataatcaacaagattcgtgtattcagctgcctgatggatacgaagacagagtcaaaaacagaggtgtaatatacaaaggatgggtaccacagacgaagatactgagtcattcgtcagtaggtggattcttgactcattgtggatggaattcggtcatagaagcgctatgttttgggcgggttttggttatgttcccggtgttgaatgatcaaggattgaatacaaggttgttgcaagagaaaggagttggtgtagagataccaagaaatgaaaaagatggatttttcactagtgattcagtggctgaagctgtgaagtttggtgtagtgagtgaagagggtgagttgttgagagctaatgcaagacaaatgagttgtttgtttggagataggaagagaaatgaacaacttattgatgattgtgttggttatttcatggaaaataggataagcaaatctaatagctccatcggttgactatctaactttttacctgagttcaattctcatcttctatttccccaaaaaaggagttaagaaagtagtcgttgcaatcgagtttcttactttttttacaccttgtttggatgattgttacctgttttattgtattgtattgttagtttaaatataatgtttgtttcaactgttatttatattctattatatggtatcgttcaaatctatcgttgttatactcattttatgtaacgatttggtgtgattgtgttgttaccttgataatttcttttcattttgtctttatttattatttaataatcatatttcatcttttatc

SEQ ID NO: 17 - DNA, 2055 bp, Solanum lycopersicum
  source 1..2055: mol_type = genomic DNA
agttatcgactcctataaaacgtggtagaaaatcattttgcatttgctctctaaattaagtagaaataacaaaatcagacgtttgtttctttggctttgaaagttcaatcactttcccattttcccggtggtccggcggctacctcatcggcaactgagaaaaatgtctgaaaatcaccctcatgttctcatctttccttatccagcacaaggacatatgcttccccttttggatttcactcatcagctagtcaacaatggagtatacatcactattttagtcacccccaaaaatctccccttcttgaacccccttctctccaggaacccatcaatcaaaactctcgttttgccttttccatctcacccttcaattcccgccggcgtagagaacgtcaaggaccttcctgctaatgggttcctttccatgatgtgcaatctcggaaaacttcgagatcccatattggaatggttccacaaccatccttcgccgccgtcagctattatttctgatatgtttttggggtttactcacgaaatcgccacccagctcggtattcgccggtacgtattctccccctccggcgcgttggccctatctgttgtttattcactttggcgtgaaatgcccaaaaggaaagacccgaacgatgaaaacgaaaattttcacttccccaatatccccaattccccgaaattccccttttggcagatctctcctatttaccggagttatgttgaaggggacccaagtacagaattcatcagagaatgctatctcgctgatatagcgagtcacggaatcgttttcaacacgtttatcgaattggaaaatgtgtatttggattatctgatgaaagatttgggtcacaacagggtttggtctgttggaccagtacttcctccgggagaagaagatgatgtttccgtccaatcaaacagaggcggttcgagctctgttttagccagtgaaatactagcatggctcgacaaatgtgaagatcactcagttgtatacgtctgtttcggcagtcaagctgtattaacaaacaaacaaatggaggaactagccattgctttggacaaaagtggtgtccatttcattttgtcagccaaaagagccaccaaaggacacgcttcaaatgattacggtgtgattccgtcttggtttgaggaaaaagtggctggacggggcctggttgttagagactgggccccacaagtgttgattttaaaacaccgagctatagccacattcttaactcactgtggttggaattcgactctcgaaagcttgacagcaggaataccattgttaacatggccgatgggtgctgaccaattcgccaatgcaaaccttttagttgacgagcatgaggtagctattagagcctgtgaaggtgtgcaaacggtaccaaattcagacgagttggctgcgctactggcggaagctgtgcaaggaaacaaggtagaagaccggagacttcgtgcatcgaagctacgaaaaattgcaattaatggcattaaagaaggcgggaactcatttaaagaattggcagcgttcgtaaaacatttaagggaggaggctacaataatcgaagcgtaaaactaactatggaggcaattaaaaattctagaaagtccaaatcattagatttatcgtttacgatacaaatatgtaagcatagattattttatggacaagtgtttgcaaatttgaaacttcaatctaaaaatatgcaattaatatgtcaagtgaaaaaatgcttctgttaataagtattttatttttcaaatataattgtttaataggagtactatattaagtcaaatcgtttttataaatcaaaccaactaccctcatttggtctgcctgaccgactattccactgttgtaggtcaggctagtggaaatatttggtctgcttttttgtattttccacaaattttctttaacttcaacatattatatggtgtagtatttttcttgttcaaacttcttagtctgttgcttttatcttttattttgaatgacggggatggtgaggaaaaaaa

SEQ ID NO: 18 - DNA, 1912 bp, Solanum lycopersicum
  source 1..1912: mol_type = genomic DNA
agtacattgtatacccaataattgtcttgtgtcattgcatatgttggcaataaaaagttggaatatagaagaggaagaagccaacacacaataatccaaattgtacacttcaactcaatcaactaactataagaatatacaacactaagttgatttttttttatttcagccatcaattgagaagttgcaagagatgactactcacaaagctcattgcttgatcttgccatatccagtccaaggtcatatcaacccaatgcttcaattctccaaacgtttacgatccaaacgcgttaaaatcactatagcactcacaaaatcctttttgaaaaacatgaaggaattgccaacttctatgtcaattgaggccatatctgatggctatgatgatggtggtcgcgatcaagcaggaactttcgtggcctatattacacgattcaaagaaattggttcggatactctgtctcaacttattcaaaaattggcaattagtggatgtcctgtaaattgcatagtatatgatccattcctcccttgggctgttgaagttgcaaaacaatttggattgattagtgctgcattttttacacaaaattgtgtagtggataacatttattaccatgttcataaaggggtaataaaacttccccctactcaaaatgatgaaaaaatcataattcctggattttcaagtccaattaaggcatcagatgtacctaattttgttattaatcctgaaggagaaagaatacttgaaatgttggtgaatcaattctcaaatcttgacaaagtagattgggtcctaatcaatagcttttatgagttggagaaagaggtaaaatattcttccgcatattcttttttatgtagatgttgaattccagatctacggtttcgtatgtttacttcttcatattttgaatctccttagtgaaaaatcctgactcaaccacccatagtgtaaaaaacattaaaggaattaactgaatcatataatatagtttattatttgtcatgtaggtaattgattggatgtcgaagatatatccaataaagacaattggaccaacaataccatcaatgtacttagacaagagactacatgatgataaagagtatggtcttagtatgttcaagccaatgacaaatgaatgcctaaattggttaaaccatcaaccaattagctcagtgttgtatgtatcatttggaagtttagccaaactaggaagtgagcaaatggaagaattggcatggggtttgaagaatagcaacaagagcttcttgtgggttgttaggtctactgaagagcccaaactccccaacaactttattgaggaattaacaagtgaaaaaggcttagtggtgtcatggtgtccacaattacaagtgttggaacatgagtcgatagggtgttttctgacgcactgtggatggaattcgacactggaagcgattagtttgggagtgccaatggtggcaatgccacaatggtcagatcaaccaacaaatgcaaagcttgtgaaagatgtttgggaaataggtgttagagccaaacaagatgaaaaagggatagttagaagagaagttatagaagaatgtataaaactagtgatggaagaagataaaggaaaattaattagagaaaatgcaaagaaatggaaggaaatagctagaaatgttgtggatgaaggaggaagttcagataaaaatattgaagagtttgtttccaagttggttactatttcctaagtaggaagttaataaataattaaagaagttgatttgtattttgcaatttctttttctaattaaagaagcaaagtttgacttgaatcttcttttattttctcctctttctattattttattattcatttattgtggaatgtaacaaacaaattgtcatggtcaa

SEQ ID NO: 19 - DNA, 1018 bp, Solanum lycopersicum
  source 1..1018: mol_type = genomic DNA
atggcttcttccaaatttccaagcataattgaaaccctaaaacctaacttgattatatatgatgggttccaaccatgggtagcaactatggcttcatcatacagtattcatgctattatgttttatgtttcttcaacttctggtcttgcctacatttaccaccaatttcttcatgggagttcaagccttacatcttttccattttcttccatataccttcatgaccatgagatcaagaaattaggcatacaaccaataaaaccacgcgatgagaaagcttttgcatacataatccttgagtcttttgaacaatctcacaacattgttttgttgaacacttgtagggagactgaggggaagtatatagattatgtttctacaataggaaagaaagagttgataccaattggaccattaattcgcgaggcgatgatagatgaggaggaggattgggggacaattcaatcttggctagacaagaaggatcaattatcatgtgtttatgtatcatttggaagtgaaagcttcttgtcaaagcaagaaattgaagagatagcaaaagggcttgagctaagcaaagttaattttatttggacaatcaaatttcctaaaggggtgaacaaaacaattgaagaaatggttccacaaggttttcttgaaagtacaaagtctaattttgaaccattcaagtattggaggttttgtaactcattgtggatggaattcgatgttagaaagcatgagttttggcataccattaatagccatgcctatgaatcatgatcaaccattgaattcaagattagtggaggaacttggcataggggtggagattttgagaggtgaaaatggagaaataatgaaagaagaggtggcaaaaggaataaagaaagtggtagaggataacactaggaaacaagttaatttgaaggcaatggaattgagtgaaaagataaaattcaaaggggagaaagctattgatgaaggagttaaaaaagggttgaagcttttgtgttga

SEQ ID NO: 20 - DNA, 1871 bp, Solanum lycopersicum
  source 1..1871: mol_type = genomic DNA
ggacatgatacgatagatattgaaattttcgatatagtaacatgctcacccctataaaatccttatatagattcctctaactatcatcatatataccacacaataattaagagtcatacaagtcaaagctttctctcacataatactaattgacctcgaactctgaacttaggttctatttttaagtttgtgagtgtaaaaatggtgcaaccccatgtcctattggtgacatttcctgcacaaggtcatatcaatccatctcttcaatttgccaagaggctaatcgaaatgggcattgaggtgacattcaccactagtgttttcgcccaccgtcgtatggcgaaaatcgcggcttccacggcacccaagggcttaaacttggcggcattctctgatggatttgatgatggtttcaagtctaacgtggatgattctaaacgttacatgtcagagataagaagtcgtgggtcccaaacattgagggatgtcattttgaagagttcagacgaaggacgtcctgtcacgtccctcgtctacactcttctgctaccttgggcagctgaggtagcgcgtgaactccatatcccatccgcgttactatggattcaaccagcaactgtgctagacatatactactattacttcaatggctatgaggatgaaatgaagtgtagtagctcaaatgatccaaattggagtatccaattgccaaggcttccattactaaaaagccaagatcttccatcttttttagtttcatctagctcaaaagatgataagtatagttttgctctaccaacattcaaagagcaactagacacattagatggtgaagaaaatccaaaggtacttgtgaatacatttgatgcattagagctagagccactcaaagctattgaaaaatacaatttaattggaattggaccattgattccttcatcattcttgggtggaaaagattcattggaatcttcatttggtggtgatctttttcaaaagtcaaatgatgactacatggaatggttaaacacaaagcctaaatcatcaattgtttatatctcatttgggagtctattgaatttatcaagaaaccaaaaggaggagattgcaaaagggttgatagagatccaaaggccattcttatgggtaataagagatcaagaagaagaaaaagaagaagagaaattaagttgcatgatggaattagagaagcaagggaaaatagtaccatggtgttcacaacttgaagtcctgacacatccatctttaggatgttttgtctcgcactgtggatggaattcgactctagaaagcctatcgtcaggtgtacctgtggtggcatttcctcattggacagatcaagggactaatgcaaagttgattgaagatgtttggaagacaggtgtcaggatgagagtgaatgaagatggtgtggttgagagtgatgaaataaaaaggtgcatagaaattgtaatggatggtggagaaaaaggagaagaaatgagaaaaaatgcacaaaaatggaaagaattggcaagagcagctgtgaaagaaggtggatcttcagaagtgaatctaaaagcttttgttctacaagttagcaaaagttgttgaaaaattatgggaaattatttttctagttgtaattttcttttcttttttcataataaaatttagagttttatatcttgatcgcataaaagaattattgtaatttactttttacaataagtaaacttacttaacctagaaaatatggcatacaaccaatcgcgttcaccctaaattcaaaattttcaatgcatatgaatttacatgaagaaaatataagaaataatactaaatctttttaaaataaattggttagatac

SEQ ID NO: 21 - DNA, 2204 bp, Solanum lycopersicum
  source 1..2204: mol_type = genomic DNA
aaaatttatgaaaactgttttctagtaaattaagtgaatttcttactttggtacgtaaataaaaaaaatatttgtatataacttagacaaatactataaaaggtgagaatgagagactattggcgtaggagtaagatgagatgtgttgaagggtgcataatatgaaaactttcatttatttttgaagagcttaaggaaaatcggaacttcattcaaacacaatatttgatacaaatacatgatttcaacaaaagttattacgttcacgtaaactaccgctactttgctagatccgcccctgcagtcctcatactatatataaacttccttcaagaaacagaaatttaagaaatcaaaagatgaaaatagaaagaaaacagagtgtagtactggtgccacacccataccaggggcatttaacaccaatgctacagcttggtagtatccttcattcacaaggcttttctgttatagttgcacatactcaatacaatactcctaattattccaatcatccacaattcgtcttccattctatggatgacggattacagggaatcgacatgtcattcccgagtttagaaaacatatatgatatgaacgaaaactgcaaggcgcctctcagaaactaccttgttagtatgatggaagaggaaggtgatcagcttgcttgtatcgtttatgacaacgtcatgttctttgtcgatgatgtagcgactcagttgaagcttcccagcattgtcctgcgcactttcagcgctgcgtatttgcactctatgatcaccattttacagcaacctgaaatatatttaccttttgaaggtatatgagaacaagtacacatcctctgcttcaatatctaaatgaaaaaaaatgaaagtagaataagttgttatttttacagtttcagtaaaatatttcggtacattttttccttgcattgattactcactacaattttcagattctcagctgctggatccgttaccggagcttcatcccttgagatttaaggatgtaccgtttcctatcatcaataatacagttccagaaccgatactagacttctgtagagcaatgagtgatataggatcatctgtcgcgactatatggaacacgatgcaagacttggagagttcaatgttgttacgccttcaagaacattacaaggtgcccttttttccaataggcccggtacacaaaatggcatctttggtctcatcgactagcatactagaagaagacaatagctgcatcgagtggctcgatagacaagcccctaactctgttctctatgtcagcttgggtagcctagtgaggattgatcacaaagagttgattgagactgcttggggattagctaatagcgatcaaccgttcttgtgggttattcgacctggctctgtctctggctttcaatgtgctgaggcactgcctgatggttttgagaaaatggtaggagaaagaggacgaatagtgaaatgggcaccacaaaaacaggtgcttgcacatcccgcggtagcagggtttttcactcattgtggttggaattctacgcttgaaagtatatgtgaagaagtccctatggtgtgcaggccatttctagcagaccaactggtgaacgcaaggtatctgagccaaatatacaaggttgggttcgaattggaggttatcgagagaacggtcatagagaaaacaataagaaaactcatgttaagtgaagaaggcaaagatgtgaagaaaagagtagcagacatgaaacaaaagatagttgctggaatgcagattgattgcacttcacataagaatctgaatgatttggtagacttcatttctgccttgccatcacgactcgctccgccaacgcctgtctttggggcaatcatgagctcaaaccacatagcaagcaagtgtatcatagagtcttgaagttatttttgagctcaaaccatatttctgtggccccttagcttggcaagacaataagtccttaatcacaaaaggaagaataagataagaagtgaacttttcataaatccatggtgcaaatgctgaagcaatttactttagtttgttgattggtttataagttgggaaccttgacatattgttttctagttggtaggaaaatattttcgtggaaaataaatgattttcaaacttattttttagtttttgattagtaaagaatattattgtcccaa

SEQ ID NO: 22 - DNA, 1856 bp, Solanum lycopersicum
  source 1..1856: mol_type = genomic DNA
agtgtaataatataacaacaaaaaaaaatattctctcatttttttcttttgatttaacaatgggtgttcttactatagagccacattttgtgttgtttcctttcatggcacaaggccatacaatccctatgattgatattgcgcgattactatcacaacgcggagttattatcacaatcgttacaacacatttgaacgcgaatagattcaagaaagtcattgatcgcgctatagaatcaggactgaaaattcaggtggttcatctctactttccaagcttagaggctggactgcctgaaggatgtgaaaatttcgacatgcttccatcgatggatttggggctgaaattcttcgatgctacggaaagacttcagccccaagtggaagaaatgttgcgagaaatgaaaccttcaccaagttgtataatatctgatatgtgtttcccatggacaactaatgttgcacaaaagttcaacattcctagaattgttttccatggaatgggttgcttttctttgttatgtttacataatttgaaagattgggaagggttagaaaagatcgagtctgatacagagtattttcgagtgcctggattgttcgacaagattgaactaaccaaaaatcagcttggaaatgctgccaggccgagaaatgaagagtggcgagtaatgagtgaaaaaatgaagaaagcggaggaggaagcttatggaatggtggtgaatacctttgaggatttggaaaaagaatacatcgagggtttaatgaatgcgaaaaacaagaaaatttggaccattggccctgtttcactctgtaacaaagaaaaacaggacaaagctgaaagagggaacgaggctgcaattgatgaacacaagtgtctaaactggcttgattcgtgggaacaaaactctgtactctttgtatgtctcgggagcctatcgcgcctttccacgtctcagatggttgagctagggcttgggttagaatcatcgagacgaccctttatttgggttgttagacacatgtcagatgagttcaagaattggctagttgaagaagattttgaggaaagagttaaagggcaaggactattaatccgcggttgggcgccacaagtactactcttgtcacatccttcaataggtgcgttcttgactcactgtggatggaattcgagcctggaaggtataaccgctggcgtggccatgatcacttggccaatgtttgctgagcagttttgtaatgagaggttaatagtggatgtactcaagacaggagtgaggtcaggcatagagaggcaagtgatgtttggagaagaagaaaaattgggaacacaagtaagcaaagatgacattaagaaggtgattgaacaagtgatggatgaagaaatggaaggtgaaatgagaagaaaaagagcaaaagaattaggagaaaaggcaaagagggctatggaggaagagggttcatctcatttcaacttgacacaattgattcaagatgtgacagagcaagcaaaaattttaaaacctatgtagaggttggtttaggatatggtgaaattaatgaattatgccatttagtaaagttcagttacttcaatataggagtaatatttaattttatggtcttataattgtattgtacacggaaagagttaaaaaagtatctttaaattacgtgaaattgaataaaaatggtgttaggtaatcgtttggttcaacaatcctcttgttattaattgtttggttttaaaatgtttgtattatcaatggtttgattctaaatgtcatttttcttaagaaatatagtagtattttattaaaaaaacatactctattcctaattaaaaagtgtca

SEQ ID NO: 23 - DNA, 1239 bp, Solanum lycopersicum
  source 1..1239: mol_type = genomic DNA
atgtctgttgccttgttatgggttgtttctccttgtgacgtctcaaatgggacaagtttcatggaatcagtccgggagggaaaccgtttttttgattcatcgaggcataggaatttggtgtccaatgagagaatcaatagaggtggtggaaagcaaactaataatggacggaaattttctgtacggtctgctattttggctactccatctggagaacggacgatgacatcggaacagatggtctatgatgtggttttgaggcaggcagccttggtgaagaggcaactgagatctaccaatgagttagaagtgaagccggatatacctattccggggaatttgggcttgttgagtgaagcatatgataggtgtggtgaagtatgtgcagagtatgcaaagacgtttaacttaggaactatgctaatgactcccgagagaagaagggctatctgggcaatatatgtatggtgcagaagaacagatgaacttgttgatggcccaaacgcatcatatattaccccggcagccttagataggtgggaaaataggctagaagatgttttcaatgggcggccatttgacatgctcgatggtgctttgtccgatacagtttctaactttccagttgatattcagccattcagagatatgattgaaggaatgcgtatggacttgagaaaatcgagatacaaaaacttcgacgaactatacctttattgttattatgttgctggtacggttgggttgatgagtgttccaattatgggtatcgcccctgaatcaaaggcaacaacagagagcgtatataatgctgctttggctctggggatcgcaaatcaattaactaacatactcagagatgttggagaagatgccagaagaggaagagtctacttgcctcaagatgaattagcacaggcaggtctatccgatgaagatatatttgctggaagggtgaccgataaatggagaatctttatgaagaaacaaatacatagggcaagaaagttctttgatgaggcagagaaaggcgtgacagaattgagctcagctagtagattccctgtatgggcatctttggtcttgtaccgcaaaatactagatgagattgaagccaatgactacaacaacttcacaaagagagcatatgtgagcaaatcaaagaagttgattgcattacctattgcatatgcaaaatctcttgtgcctcctacaaaaactgcctctcttcaaagataa

SEQ ID NO: 24 - DNA, 945 bp, Solanum lycopersicum
  source 1..945: mol_type = genomic DNA
atggcggccggaatttcagcctccgctagttcccgaaccattcgcctccgtcataacccgtttctcagtccaaaatccgcctcaaccgccccgccggttctgttcttctctccgttaactcgcaattttggcgcaattttgctgtctagaagaaagccgagattggcggtttgttttgtgctggagaatgagaaattgaatagtactatcgaaagtgagagtgaagtaatagaggatcggatacaagtagagattaatgaggagaagagtttagctgccagttggctggcggagaaattggcgaggaagaaatcggagaggtttacttatcttgtggcagctgtgatgtctagtttggggattacttctatggcgattttggcggtttattacagattttcatggcaaatggagggtggagaagtgcctttttctgaaatgttagctacattcactctctcgtttggcgctgccgtaggaatggagtactgggcgagatgggctcatagagcactatggcatgcttctttatggcacatgcacgagtcgcaccatagaccaagagaaggaccttttgagatgaacgacgttttcgccataacaaatgctgttccagctatagctcttctttcctacggtttcttccataaagggatcgtccctggcctctgtttcggcgctggattggggatcacagtatttgggatggcttacatgttcgttcacgatggactggttcataagagatttcccgtagggcctattgccaacgtgccttactttcggagggtagctgcagcacatcagcttcatcactcggacaaatttgatggtgtcccatatggcttgttcctaggacctaaggaattggaagaagtaggaggacttgaagagttagaaaaggaagtcaaccgaaggattaaaatttctaagggattattatga

SEQ ID NO: 25 - DNA, 2295 bp, Solanum lycopersicum
  source 1..2295: mol_type = genomic DNA
atgtggaagttgaagattgcaaaaggacaagatgatcgatacttgtatagtacaaataactatattggacgacaaatatgggagttcgatccaaatgctggaacaatagaagaacaagccaaaatcgaagaggcacgtcaacattattggaacaatcgttacaaagttaagcctaatagtgatcttctttggagaatgcagtttttgagagagaagaatttcaaacagagaattggagcagtaaaagtagaagaaggagaagaaattagccatgaaattgctacaattgcattgcgtagagctgttcatttcttctcagctttacaggctactgatggacattggcctgctgaaaatgccggacttctcttctttcttccacctctcgttatgtgtatgtatatcactgggcatcttaatactgtatttccagctgaacatcgaaaggaaattcttcgttatatatactgtcaccagaatgaagatggcggatgggggttgcatatagaaggtcacagtacaatgttttgtacagctctgagttatatttgcatgaggatccttggagaaggaccagacggtggcgaaagaaatgcgtgtgctagagcgaggaaatggattcttgatcatggtagcgttactgcgattccttcttcggggaaaatatggctctcgattcttggagcttttgagtggttaggaaccaatccaattcctcccgagttttatattcttccttcttttcttccaatgcatccagcaaaaatgtggtgttacagtcgaatattttacatgccactgtcttatctctatggtaagagatttgttggtccaatcacacctctcattttgcaattgagggaagagttatatgatcaaccatacaatgaaatcaactggaaaagagtacgccatctatgtgcaaaggaggatctctattaccctcatccactggttcaggacttgatgtgggacactctctactttttttacgagcctttattgactcgttggcctttcaacaagctgagaaataaagctcttgaggttacgatgaaacatcttcattatgaagacgagaatagtcgatacatcactgttcaatgtgtcaataaagtactgtgtatgcttgcttgttgggttgaggatccaaacggcagttatttcaaaaaacatcttgctcgggttccagattatttatgggtagctgaagatgggatgaaaatgcagaatagcggtagtcaatcatgggatactagttttgctattcaagcactattggccagtgagatgaacgatgagatatcagatactcttagaaaaggacacgacttcataaaaaaatctcaggtaaaggataatccttctggtgattttagagggatgtatcggcatatttcaaaaggatcgtggactttttcagatcaagattatggatggcagtcatctgataccactgccgaagcattaaggtgctgcctttttttctctacaatgccatctaaattagttggtgaggcaatggaacctgcacgactgtatgactcggtgaacgttattctttccttacagagcaaaaatggcggtttagcagcatgggagcctgcagggacctcaaagtatttggagttgctcaatcctactgaaatatttgaggacgtcgtcatcgagcatgagtatgttgagtgcactggctcatcaatacaagcacttgttcattttaagaagctataccctggacaccgaaccacggagatcgacaatttcattgataatgctgttaaatatattgaaaatgtacagaggcctgatggttcatggtatggtttctggggtgtgtgctttacatatgcttcctggtttgctcttggagggcttgctgcagcaggcaagagttacagcaactctgcagctgttcgtaaaggcgttgactttctgctacaaagacaaaggtctgatggtggttggggagaaagctaccattcttgtcccgacaaggtatatagagagcttgaaacagaacactcgaatcttgtacaaactgcatgggcattgatgggattgattcactctggccaggctgatagagatccaaaacccctccaccgtgcggcaaggcttttgattaattctcagatggaagatggtgacttcccacaacaggaaataactggagctttcatgaagaattgcatgttgcactatgcaacatttagaaatatatttccattgtggggtttggctgaataccgcaaaaatgtttcacctactaaacttgaagaagatattaaaaag

SEQ ID NO: 26 - DNA, 1842 bp, Solanum lycopersicum
  source 1..1842: mol_type = genomic DNA
atgtggaagttgaagattgcaaaaggacaagatgatcgatacttgtatagtacaaataactatattggacgacaaatatgggagttcgatccaaatgctggaacaatagaagaacaagccaaaatcgaagaggcacgtcaacattattggaacaatcgttacaaagttaagcctaatagtgatcttctttggagaatgcagtttttgagagagaagaatttcaaacagagaattagagcagtaaaagtagaagaaggagaagaaattagccatgaaattgctacagttgcattgcgtagagctgttcatttcttctcagctttacaggctactgatggacattggcctgctgaaagtgctggtcctctcttctttcttccacctctcgttatgtgtatgtatatcactgggcatcttaatactgtatttccagctgaacatcggaaggaaattcttcgttatatatactgtcaccagaatgaagatggtggatggggtttgcacatagaaggtcacagtactatgttctgtacagctatgagttacatttgcatgaggatccttggagaaggaccagaaggcggtgttaataatgcgtgtgctagagcaaggaaatggattcttgatcatggtagtgtcatcgccattccttcttggggaaaaacatggctctcgattcttggagctttcgaatggataggaaccaatccaatgccacctgagttttggattcttccatcttttcttcccgtgcatccagcaaaaatgtggtgttactgccgaacggtctacatgccaatgtcttatctctatgggaagagatttgttggtccaatcacacctcttattttgaaattgagggaagagctgtatgatcagacatatgatgaaattaactggaaaaaagtgcgccatgtatgtgctaaggaggatctttattacccgcatccttttgttcaagatttgatgtgggatagtctctacatatgtaccgagcctctattgactcgttggcctttcaacaagttgagaaataaagctcttgaagttaccatgaaacacatacactatgaagacgagaatagtcgatacatcaccatgggatgtgtggaaaaagtattgagtatgctcgcttgttgggttgaggatcccaatggcgatcatttcaaaaaacatcttgctaggatcccagattttttatgggtagctgaagacggaatgaaaatgcagggttgtggtagtcaatcatgggatgctagtcttgctattcaagcgctattggccagtgagatgaatgatgagatatcagatactcttaaaaatggacacgactttataaaacaatctcaggtgaaggacaatccttctggtgattttaaagtgatgtatcggcatatctcaaaaggatcatgggcttttgcagatcaagatctcggatggcaagtatctgattgcactgccgaagcgttaaagtgctgccttctcttctctacaatgcctcctgaaatagttggtgaggcaatggatccagtgcgactgtatgactcggtgaatgttattctttcattacagagcaagaatgggggtttatcagcgtgggaacctgcaggggccccagagtatttggagttgttaaatcctactgaatttttcgaggatattgtgattgagcacgagcatgttgagtgcactagctcggcaatccaagcacttgttcgttttaagaagctataccctggacaccgaaccacggaggttgacaattttattaataatggtgttaaatatattgaagatgtacaggagcctgacggttcatggtatatgatatttctctcgtga

SEQ ID NO: 27 - DNA, 1554 bp, Solanum lycopersicum
  source 1..1554: mol_type = genomic DNA
atggaaatctggtttatcgttgttgttactctctgtgtagctttctggttgaaatccatttgtaatattattgtctctccaaattccaataccaagagaaacctcccgccgggaccctacagttttccggtgatcggtagtttattatgggccaaacggaccttctccgacttggaacccatcctccgtgaccttaaagctaagtatggtccgctgattaccctaaatattgggtctcgttcagccatattcgtagctagccactccttagcctaccaagctttagtccagcaaggtgctattttctctaaccggccaaaggctgggcctaccagtgcagtcgtcaatagtaaccagcggaacatcagctccgccccttacggccctatatggcgtctactccgtcgaaatttgacttcagaaatactccacccatctcgtatcaagtcatattccaaggccagatcttgggtgctgggtatcctcattcaacagctccgtaacgcccaagttgattctgtgaagttaattgatcattttcagtatgcaatgttctgtcttcttgtattgatgtgttttggggataaacttgaggaaactcaaatccgacaaattgaaaatatacagcgcaagttgctcctgggtttcacacgattcaatataataaatatatttcctagagttggaaaaataatttttaggaatcgctggaaggaactcattgaactacgacaagagcaagagagtatcatcattcctttgattgaagctcgaagcagggccagagaacaaaggaccgagcacggtgatgaatttgtggtggcttatgtggatacactgttgaatttggagttcccagaggaaaaaaggaatctcaatcatggagaaatcgtaacgctctgtagtgaattcctaagcgccggaactgatacgacgtccactgccttacagtggattatggccaacttggtcaaaaacccttccattcaggaaaaactataccaagaaattgccactgtagtgggagaaaaacagagcaagttgacagatgaagaggtggtgaaggaggaggatttgcagaaaatgccctacttgaaagcagtgatattagaaggtcttaggcggcacccacctggtcactttgtgctgccacacacagtgacggaggaagtagaactgaacggctacgtcatcccaaaggatgtcaccatcaatttcatggttgcagagatgggtctagacccaaaggtgtgggaggatcccttggagtttagaccagagaggttcttagtggaaggatcagataacgaagggtttgatataacagggagtagagagatcaagatgatgccattcggcgcagggaggagaatatgtcctgcctatgcattggctatgcttcacttggaattctttgtggctaatttgatttggcattttcaatggaagcctgtggaaggagatgatgttgatctcacagagattctagaattcaccgtagttatgaagaacccattacgagctcgtatctgccccagagttaactccgtttga

SEQ ID NO: 28 - DNA, 1521 bp, Solanum lycopersicum
  source 1..1521: mol_type = genomic DNA
atggagtttccttggtattctgttgttgttcctctgattgttttccttttctttcttcataactgcttatttaccatttttaatacgagtaacaaaaaattaccaccatctccaagaaagctcccaatcataggaaatttgtatcaacttgggttgcatcctcaccgttctctctataaactatcgaaaaaatatggtccagtcatgctacttcacttgggcagtaaaccagttctggttgcttcctctgttgatgctgctcgcgatatcttgaaaactcatgatctcgtatggtcaaccagaccaaaatctagcatcgctgatggactcctttatggatccaaaggtgtggcctttagtaattatagtgaatattggagacaagttagaagtgtcatcgtgcttcaccttctcagcaacaaaagagtacaatcttttcgtgatatcagagaagaagaagtatcaaacatgattgatgaaatcaggaaaaggagtactagttcttcgaattcagtgatagatatgagagatgttttaagttgtatgactaataatataatcaaccgagtgacaataggaaggacatacaatgaaggggaaagtggaagagctgttaaggctctattagaagatcttcttgcacttttaggtacttttaacattggggagtatattccatggcttaaatggctcaataaaatcaatggtcttgacaacagagtgaagaaagtagctaaagatttggatgcatttttagacagcgtgattgaagaacgcgtggttagaaacaaaaaagcagaaaacagtacgggtgaggctaaagatttcgtggatgttttgctggaaattcagaatggaaacgaaactggctttccgcttcaaagggattcattgaaagctattctcttggactcgtttattgctggtgtggattcaatatatacaactttagagtggataatgatagagcttttgaggaatccaagagccatggagaaattacagaatgaagtgcgaggattagtccaagggaaagcagagataacagaggacgacttaggaaatatgttgtatctcaaagcagtaatcaaagagagtctcaggcttaatccaccgtttccaataccagttcctcgagaatcaatggaagacgtaaaattactagactataacatacctgctaaaactcaagtccttgttaatatttgggcaatcggaagagacccattatcatgggatgaaccagaggagtaccggcctgaaagattcttgaatagtgatattgatttcagaggactaaactttgagttgattccgttcggagctggcaggaggggttgcccaggaattccttttgctattgtgataattgagctaacattagcaaggcttgtgaacaaatttaattttgcattaccacaaggaataaaaaaagaggatttggatatgagtgaatgcactggcatctccattcgcaggaaattgcctttactagcagtggcaactccgtgcagtgtttag

SEQ ID NO: 29 - DNA, 1560 bp, Solanum lycopersicum
  source 1..1560: mol_type = genomic DNA
atgttatacaccacaccctctaatcttaccagacgtgtttggtttaatattgaaagtattttctcttcttattcaatgactttgatatggacaattctagtggttggtctatctgtatatgccttgtatgagctgctaaacattcacaagagaaaaaggtatcctccaggtccaacagggcttcccattttaggacatcttcatttgttaggcaaaaatccacacaaagatttacaaaatctagcccaaaaacatggtcctattatgtatatgagactagggctagtacctacaatcattgcctcatctgctgatgcagccgagaaggtcctcaagacatatgatcacatctttgctagtaggcctcatcatgaggcatctcaacatttggcttatggccaaaagaatttggtttttgcaaagtatgatgtgtattggcgcaacataagaaaattgtgcacagtgcaccttttgagcaatcaaaagatccattcatttcaatccatgagaaaacaagaggttgaacttcttattgagtcgcttaaacaggaagctcgtgatcgcgtagttattgatcttagtgcaaaggttacgtccttgaatgctaacttgacttgcttaatggtgtttggaaagaagtacatggatgaggacttggacaagaggggattcaaagctattgttcaagatgtcgtgcatttagcaggtatgccaaatcttggtgattttttccccttccttggtgctattgatctccaaggtatcactcgcaagctcaaggatctttcaaaggtgtttgatgagttcctggagaagattattgatgaacatgtttacgctcatgaacataagcaaaacaaggactttgtggacaccatgatggacataatgcaatctggagatgcaaaatttcagtttgaccgtcatcacataaaagctatcctttttgacatgcttatagctgcaatggacactacagcatcatcaacagaatggatactcaccgaacttcttaggcacccccaagtgatgaaggaactccaaaaggagttgcaagaagtggtaggcctcgatagaatggtggaagaatctgacctggagaatttaaagtacttagacatggttgtaaaagagggcctgaggctgcatcctgtagcgccactattttatcacgagtccatagaagattgtgtagtcgatggtttccacatacaaaagggaaccagaatcataataaattgttacgcgattcatatggatccaaatgtttggcctgatcctgagaagtttttgcctgagagatttgttgggagcagtgtagattttcgtggacgggacttccaacttgtaccatttggctctgggagaagaagctgccctggaatgcagttggcagttatggtcgtccgccttgtggtggcacaattggttcattgctttgagtgggagcttccaaatggtattgagtcttgtgatttagatattgatgagaagtttgggatagtaacatgtagagaaaaacctttgctggctattcctacatataggctaaacaaatga

SEQ ID NO: 30 - DNA, 1434 bp, Solanum lycopersicum
  source 1..1434: mol_type = genomic DNA
atggacttaatcatttttattgtaatgttgttagtgatggttttagtagtagttttattagttctattaagaacaaataaggcttctccaaaagaaatggaagcaattccaggtaccttaggatggcctattattggagaaagtctctctttcatatccgaattttcgagccctgctggtatatacagcttcatcaacaagagacagaaactgtatgggaaggtgtttaagagttatgtgttgggaaggtatacagtattcatgacgggaagagaagcaagcaagatcttgttgacaggtaaagatgggatagtgagcttaaaccttttctacacaggacagcaagtacttggacctacaagcttgctccaacagacaggagaggcgcacaagagactccgaagactaattgctgaaccactttctgttgatggtttaaagaagtattttcagtttatcaacagtttagctattgaaacattggatcagtggtctggaagaaaaattttggttcttgaagaaacttccacatttacactgaaggtgataggcaacatgataatgagtttagagccaactggtgaggagcaagagaaatttcgaaccaatttcaagattatttctggctcttttgcttctttaccctttaaagtcccgggaactgccttttatcgtggcattcaggctcgtgatcgaatgtatgctatgttagactcgataattgaccaacggaggagtggggaaaacataaaacaagatttcttgcaatccctggtgaagaagcatggcaaagatgcacccgaaggagatgatgatgacaaactcacagataaacaacttaaggataacatattgacgcttctagttgcaggccacgatactacaactgccgctttaacatggctcctcaaatttcttcaagaaaatcctgctgtattggaacgactaagagaggagcatagagaaatccaagctcaaaaacaaggcacgttagatctcacctggtctgaagtcaacaatatgccttacaccgccaaagtaatcagtgaaacacttcgaatggccacaatccttccatggttttcaagaaaagcagcacaagattttgagattgaggggtgcaaaatcaagaagggatggtcacttaacttggatgtagtgtctattcatcgcgaccccaaaatatttcctaatccggagaagtttgatccttctagatttgatgatcctctaaaacctttcagcttccttggatttggaagcggaccacgcatgtgtcctggaatcaacttagccaaactggaaatttctgtctttctccatcatcttgtctgcagatataaatggacaccgttagacacagatgactctgtccagccaacacttgttaggatgctgaagaacaagtatcccgtcatggttgagccactttag

SEQ ID NO: 31 - DNA, 1554 bp, Solanum lycopersicum
  source 1..1554: mol_type = genomic DNA
atggagattttgtataacacaattatagcaacaatttgtgttgcaatattgttggtatatacatggagagtgttgaattgggcatggtttaggccaaaaaaattggagaatttcttaaggcaaagaggtctaaaaggaaatccatataagttactctatggagatttgaatgaactcacaaaaagtatagttgaagctaagtctaagtcaatcaatatctctgatgatataactcaaaggcttatcccttttttccttgactccatcaacaaaaatggtaaaagttctttcatgtggctaggcccatatccaacagtgttgatcacaaatcctgaacatgtaaaagagattttgacaaagaattatgtgtacctaaagcaaactcatccaaatccatttgcaaagttattggctcaaggtcttgtgttggttgaggaagacaaatgggcaaaacacagaaaaataatcaatcctgctttccatgttgagaagttaaagcatatgttgccagcattttatatgagttgtagtgaaatgataagcaaatgggaggatattgtttcaaaggaaacatcatacgagctcgatgtatggccagaccttcaaataatgaccagtgaagttatttctcgaaccgcatttggtagtagctatgaagaagggagaatagtgtttgaacttcagcaagaacaagctgagcatataatggacataagtcgttcaatatatataccaggatcgaggttcttgcctactaaaaggaacaaaagaatgctcgaaatcgaaaagcaaattcaaacaacgattaggcatatcatcgacaaaagattgagggcaatggaagcaggggagactagtaaagatgatttattaggcatattacttgaatccaatatgaaagaaattgaacaacacggaaacaaagatttcggattgacaacaacagaagtgattgaagaatgcaagttattctattttgctggacaggagaccacttcagtgttgctcgtgtggacgatggttttgctatgcctacatccagagtggcaagtacgggccagagaggaggttttacaggtcttcggaaatgaaaaaccagatttggaaggactaagtcacctcaaaattgtgacaatgatcttatacgagacattaaggctattccccccattaccggtatttagtagaaggaacaaagaagaagtcaaattaggggagctgcagctaccagctgaagtgatacttattatacctgcaatctttattcattatgacaaggaaatatggggcgaagatgcgaaggaattcaaaccagaaagattcagtgaaggagtgtcaaaggcaacaaaaggtcaagtctcgtttattccgtttggttggggacctcgaatttgcatcggacaaaactttgcaatgatggaagcaaaaatggcaatagcaatgatactacaaaagttctcctttgaactctctccgtcttatacgcatgctccatttgcaacaattactattcatccacagtatggtgctcctctgcttctgcgcaaacattga

SEQ ID NO: 32 - DNA, 1545 bp, Solanum lycopersicum
  source 1..1545: mol_type = genomic DNA
atgatagtaacaatattttgttgtgccatagcaattactttgtttgtgtgtctatggaaagtactaaattgggtttggttccaaccaaagaagttggagaaattgttgaggaaacaagggctaaatgggaattcatacagatttttgtatggggacatgaaagatttttctaagatgattaaggaagctaattcaaagcctatgaatatggatgatcatgatttagcaccaagaatggtgcctttctttcttgaaaccatcaagaaatatgggaaaaaatgttttacatggatgggtccaagaccacaaatactaatcatggatcctgagcttataaaggaagtactttcgaaaacctacgtgtatcaaaagcctcatggaaatccgttaggcacgttatttgtacaaggattagtaagctatgagaaagataaatgggccaaacatagaaaaatcatcaatcccgcgttccatctagagaagttaaagcatatgcttccagctttttacttgagctgtagtgagatgctgagcaaatgggaagacgttgttcgagttgaaggctctcatgagatagatgtatggcctcacctgcagcaattgacttgtgacgtgatctctcggacagcttttggcagtagttatgaagaaggtagaaagatttttgaacttcaaaaggaacaagctcagcattttctcgaagttatacgctcagtttatatcccaggatggagatttttaccaacaaagaggaacagaagaatgaaggaattaaaaaatgatgttcggtcttcaattagaggtattattgataaaagattgaaggcaatggaagcagggaacgcggataatgaggatctattgggcatattgctggaatcgaattttaaagaaattgaactgcacggtaacaaggactttggaatgagtatcgaagaagtcattgaagaatgcaagctgttctattttgctggtgcagaaactacatcagtgtggattctgtggacgctggtgttgttgagcagacatccagattggcaggaacgggccagagaagaagtattgcaagtgcttgggagtcgaaaaccagattttgatggattaaatcatctaaaagttgtaacaatgatcttgtacgagtcgttgaggttatattccccaataacagtacttacgcgacgagtttatgaagacattacactaggagaagtatctctaccaactggtgtgctagtctcattgccaatgattttattgcatcatgataaagatatatggggtgaagatgcaaccaagttcaatccagagagatttagtgaaggaatatcaagtgcaacaaaaggtcaagttacatattttccatttgcttggggtccaagaatatgcattggacaaaattttgccatattagaagcaaagatgactctgtgtatgatcctacaaagcttctcttttgaattgtctccatcctacacacatgctcctcaatccttggtaaccactcagcctcagtatggtgctccacttattttccacaaactataa

SEQ ID NO: 33 - DNA, 1560 bp, Solanum lycopersicum
  source 1..1560: mol_type = genomic DNA
atggacgaaattcaaatattagtaagagtgtgttgttctgccatagcaattgctctgtttgtgtgtctgtggaaagtactaaattgggtttggctcaatccgaagaagttggagaatttgttgaggaaacaagggctaaatgggaactcttacaaaatattgtatggggatttgaatgattttattggaatgattagggaagctagttcaaaacctatgaatttgtctgatgatatagctccaagattggtgcctttctttcttgagaccaccaagaaatatgggaaaaaatgttttatatggttgggtccaaaaccacaagtaataatcatggaccctgagcttattaaggaagtactctcgaaaacctatttgtatcaaaagcccggtggaaatccatttgcagcattattggtacaaggactagcaacctatgaagaagataaatgggccaaacatagaaaaattatcaatcccgctttccatctagagaagcttaagcatatgctcccagcttttcacttgagctgtactgaaatgctgagcaaatgggaagatgctgttccacttggcagctctcgtgagatagatgtatggcctcaccttcagcaattaacttgtgatgtaatctctcggacagcttttggaagtagttatgaagaaggtagaaagatttttgaacttcaaacggaacaagctcagaattttatcgacgctgtacgtgaagtttatatcccagggcggagatttttgccaacaaagaggaacagaagaatgaaggaaataaaaaacgaagttcggacctcagttaaaggtattatcgataaaagaatgaaggcaatgaaagcagggaatgccgataataatgaggatctgttaggcatattgcttgaatcaaatttcaaagaaattgaacagcacggtaacaaggattttggaatgagcatcgaagaagtcatcgaagaatgcaagttattctattttgctggccaagaaactacatcagtcttactcgtgtggactctggtgttgttgagtcggtatcaagattggcagacacaggccagagaagaagtcttgcaagtgtttgagagtcggaaaccagattttgatggattaaatcatctaaaagttgtaacaatgatcttgtacgagtcattacggctatatcccccattgataacacttaatcgacaggttaacgaagacatagtattaggagaattgtgtctaccagctggtgtactagtctcattgccaatgatcttattgcatcatgataaagatatatggggtgaagatgcaaacgagttcaatccagagagatttagagaaggaatatcaagtgcaacaaaaggtaaagttacatattttccattttcatggggtccaagaatatgcattggacaaaattttgccatgttagaagcaaagatggctttgtgtatgatcctacaaagcttctcttttgaattgtctccatcttacacacatgctcctaaatccttagtaaccatgcagcctcagtatggtgctccacttattttgcacaaactttag

SEQ ID NO: 34 - DNA, 1431 bp, Solanum lycopersicum
  source 1..1431: mol_type = genomic DNA
atgatgataatagacttagagttttctactctactcatttgccttgttccattcctttttatcttcttcatcaatcttttccattccaaatcaaccaaaatcccaaaatcttatccaatcattggttcatatttctcaattttagcaaacaaacatcgtcggattcaatggagttctgatataattcaaacaactaccaatcttactttcactctcattcgtcctttcggtcatatgtttaaatcaaccatggctgattttcttggtgatggtatttttaatgtggatggtgaaatctggaagtaccaacgacaagttgctagtcatgaattcaatactaaatctctacgaaaattcatcgaaactgtcgtagatgctgaacttaacgaacgtctgattccacttcttgctgttgctgcttctgagaaaaaagttgttgatctccaagacatattacagaggtttgctttcgataacatttgcaaaattgcgtttggatatgatcctgcttatctatcgccgtctctaccacaagagaaattcgcagttgcttttgaagaagctgttaaattaagcagtgaaagattcaatgctattttcccctttttatggaagattaaaagacatttcgatttcggaagtgaaaagagattcagaatcgctgtagatgaagttcgtcaatttgcgaaacagctcgttagagaaaaacagagtgaactaaatcaaaaatcatctctcgattcagtagatctattatccagattcttaagcgttggtcatagagacgacgatttcgttacagatgtagttattagtttcatattagcaggtcgtgacacaacatcagctgcattaacttggttcttctggttaatctcggaacaccctgtggtggaaagttcaattctaaacgaaatcaaattaaaatcccacacgccagtatacgaagaagtgaaagagatgatatacacacatgcttcactttgcgaaagcatgaggttttatccaccagtaccaattgactcgaaaacagctacagaggacaatgttttaccagatggaacttttgttaaaaagggaacaagagtaagttatcatccctacgcaatggggagagcagaggaactatggggaagtgattggaacaagtttaaaccagaacggtggttggataaagatgaagttactggaaattggaattttgttactaaagacatgtatacatatcctgtttttcaggcggggccgaggatttgtttggggaaagaaatggcgtttttgcagatgaagagggtggttgctggtgttttacggcggttcaaggtggttccggtcacggaaaaaggggtggaaccggtgtttatttcatacctcacggcgaaaatgaaaggtggtttccatgtgacaattgaggaaagggaccatgagtag

SEQ ID NO: 35 - DNA, 1512 bp, Solanum lycopersicum
  source 1..1512: mol_type = genomic DNA
atgaatcatcttgatgttttttggtggctgaagtcttttcatgtctattctattcttatcttcatcataatttctctattttctctaatattttacatggtgagggtaaaactttggtgcaattgtgaaatttgccatgcttacatcacaacaagttggtcttcacaattcatcaatttgtgtgattggtatacacatcttttaaaagaatcaccaacaagaacaatacacatacatgttcttggcaacatcataacttcaaatactcaaaatgttgagtatatgctcaaaacaagatttgataattatccaaaagggaaaactttctctactatcttgggtgattttcttggtagaggaatatttaatgttgatggggatttatggagatttcagaaaaggatgtcaagtcttgagcttggaaaagtttctataagatcctatgcatttgaagtggtgaaaaatgagattgacaaaaggcttgttccacttttacatgaatataaacaaggtggcggggttcttgattttcaagatgtttttagaagattttcatttgattgtatttgtagattctcttttggattagaccctaaatgtttagaatcctcactccctatgtcacaatttgctctttcctttgacctagcatcaaaattatcagctaaaagagctatgacaacatcacctatagtttggaaaatcaagagatttttcaacataggaagtgaaaaagagttgagggaagcaataaaagtgatcaatattctagcacaagaagttataaggcaaaagagaaaattagctttttcaaatcatagggatcttctttcaaggttcatgggatcaataagtgatgaaacttatttgagagatattgtcataagctttctactagccggtagggacactatagcatccgcgttaacaagtttcttttatgtgattggaaacaatccacaagtcgctaaagccattagagaagaagctaatcgagttctaggtccaaatgaagatctcacaagttatgaacaaatgagtgagcttcattatctacaagcatgtgtttatgaaagtatgagactttaccctcctatacaatttgattcaaagttttgtttagaggatgattttttacttgatgggacttttgtgaaaaagggaacaagggttacttatcatccttatgctatgggaagaatggaagaattatggggttgtgatgctttggagtttaagcctcaaagatggttgaaagatggggtttttgtccaagaaaatccattcaagtatccagtatttcaagctggacttagggtgtgtttgggtaaagaaatggcacttgttgaggttaaaagtgtggctctctcattactaaggagatttcacgtcgagttagctcaaccatatcaccacaaccctcgattctctcctggccttactgcctctttcaaagcatattcaacaaaccctaatcatggaggatggaactcatttcaacatatcaattaa

SEQ ID NO: 36 - DNA, 1412 bp, Solanum lycopersicum
  source 1..1412: mol_type = genomic DNA
atgggtcagctacattttttcttctttcccatgatggctcaaggtcatatgatacctacacttgacatggcgaagcttgtcgcttgtcgtggtgttaaagccactataatcacaacacctctcaatgaatctgttttctctaaagctattgagagaaacaagcatttaggtattgaaattgatattcgtttactaaaattcccagctaaggagaatgatttgcctgaagattgtgagcgtcttgatcttgtaccttctgatgacaaactcccaaacttcttaaaagctgcggctatgatgaaagatgaatttgaggagcttattggagaatgtcgccctgattgtcttgtttctgatatgttccttccatggactactgatagtgcagccaaatttagcataccaagaattgtattccatggaactagttactttgcgctttgtgttggcgatagcatcaggcgtaataagcctttcaagaatgtgtcatcggatactgaaacttttgttgtaccggatttgccacatgaaattaggctaactagaacacagttgtctccgtttgagcaatcggatgaagagacgggtatggctcccatgattaaagctgtgagggaatcggatgcgaagagctatggagttatattcaatagcttttatgagcttgaatcagattatgttgaacattacactaaggttgtaggtagaaaaaattgggctattggtccgctttcgctgtgcaatagggatattgaagataaagcggaaagagggaggaaatcatctatcgatgaacacgcgtgcttgaaatggcttgattcgaagaaatcaagttccattgtttatgtttgttttggaagtacagcagatttcactacagcacagatgcaagaacttgctatggggctagaagcctctggacaagatttcatttgggttatcagaacagggaatgaagattggctcccagaaggattcgaggaaagaacaaaagaaaaaggtttaatcataagaggatgggcaccccaagtgctgattcttgatcacgaagctattggagcttttgttactcattgtggatggaactcgacactggaaggaatatcagcaggggtaccaatggtgacatggccagtatttgcggaacagtttttcaatgagaagttggtgactgaggtaatgagaagtggagctggtgttggttctaagcaatggaagagaacagctagtgaaggagtgaaaagagaagcaatagcaaaggcgataaagagagtaatggcgagtgaagaaacagagggattcagaagcagagcaaaagagtacaaagaaatggcaagagaagctattgaagaaggaggatcatcttacaatggatgggctactttgatacaagacataacttcatatcgtta

SEQ ID NO: 37 - DNA, 1401 bp, Solanum lycopersicum
  source 1..1401: mol_type = genomic DNA
atggctgatgatcagctccatatttttttctttccattgatggcgcacggccacataattcctactttggatatggcgaagttgttcgtttcacatggtgcaaaggcaactattatcaccactcctctgaacttacccatcttcgttcaatccagtgaaaaaatcaaacatttggggctggaaatttatgtcaaagctatcagattcccagcagtggaagctggtttgcctgaaggatgtgaaagaatggatcaattgacttctgatgatttggtacccaaattcttcaaggccaccaccatgcttcaacaacaactagagctattactccatgaatgtcaacccaacgctctcgttgctgacatgttcttcccctgggctacagaatctgctgccaaatttgatatcccaagattggtatttcatgggatgggcttctttgcgctttgtgctttggagaaccttaggcattacaagcctcagaagaatgtgtgttcggatacagaacctttcgttgtcccgaatcttccccataaggtaaagctcacaaggatgaaattagctccgtatgatcgagaagaaacagaaatgactaagtttattgaacaagtgaagggatccgagttcacaagttttggggttattgtcaatagcttctatgagcttgaaccagattatgctgattactacagaaaggttttgggtaaaagatcatggcacgttggaccctttttgctctgcaacagaaaaaacgaagaaaaaatccacaaaagcgatgataaacatcatgagtgcttgcaatggctggacacgaagaaacaaaattcagtaatctatgtttgtttcgggagcatgtctaactttactgattcccaaatcgaagaaattgcattaggcctcgaagcttcagagcaagaattcatatgggtaattaggaaaggcaagattccagaaggattcgaagagcgaacaaaagaaaaaggattcatcataagaggatgggcaccccaaacgctgattcttgatcacgaggctataggagcctttttaactcactgtggatggaattcgacacttgaaggcgtatcagcaggggttccaatggtgacttggcctatatttgcagagcaattcttgaatgagcaattgataactaaagtactacaaataggagtggcagttggggcagaggaatggagcatagtggtggataatgtgaaaagtgaagcaattacaaaggcagtgaagtgtgtaatggctggggaagaatcaatggaaatgaggaggagagcagaagaagttaagaaaatggcgaacaaggctgttgaagaaggtggatcatcttacagtgatttgaatgcactgtttcaagaactaagaaccattaatttgcacaactaa

SEQ ID NO: 38 - DNA, 1440 bp, Solanum lycopersicum
  source 1..1440: mol_type = genomic DNA
atggagaatgaaaaagataaacttcatgttgtgatgtttccatggctagccatgggacatatcatacctttttttcatctatcaaagtgtttagcacgaaggggtcatagagtcaccttcatttcaactccaagaaacattgaaagagtcgtcaataaagttccctctgatcttgctcatctgttgcatattgtgtgttttcatttgcctaaagttgaaaacttgccggatgatgcagagtcatcaatggacataccttaccaaaaagctcagttcttgaagatagctttcgattcgcttgaaatcccattaaccaattatcttgagagttcatcaaatcccaaaccagattggattgtctatgactatgcttctcactggctacctcaaattgcaggtaaattaggcgtttctcgcgccttttttagtctgtttactgctgcaactatgtcattttatggacctccttgtgctttgttaaatgatgaaaggtcaatagctgaggactatacagttgttccaaaatggattcctttcgaaacgaaagttgtgtataggctgcatgagattaagaagaatttcgaagcaccagctgatgaatcaggtacatctgatggagccagatttggtgcttcaattgatgcaagtgatgtcatattgtttcgaaattgtgttgagtttgaatccgaatggttcagcttggtttctgagctttatcagaaacctattatatccattggtgttctacctccatctgttgtagttgatcatgaacaagatgacagcaatgatgcgtcgtggtcaggaatcaagaattggctagataagcacaatcaagattctgttgtttatgttgcacttggaactgaagcaacactgaatcaacaagagctgaatgagctagctttggggttagaaaaatgcggattaccattcgtttgggtattaagagatcagccaaaacataatcaacaagattcgtgtattcagctgcctgatggatacgaagacagagtcaaaaacagaggtgtaatatacaaaggatgggtaccacagacgaagatactgagtcattcgtcagtaggtggattcttgactcattgtggatggaattcggtcatagaagcgctatgttttgggcgggttttggttatgttcccggtgttgaatgatcaaggattgaatacaaggttgttgcaagagaaaggagttggtgtagagataccaagaaatgaaaaagatggatttttcactagtgattcagtggctgaagctgtgaagtttggtgtagtgagtgaagagggtgagttgttgagagctaatgcaagacaaatgagttgtttgtttggagataggaagagaaatgaacaacttattgatgattgtgttggttatttcatggaaaataggataagcaaatctaatagctccatcggttga

SEQ ID NO: 39 - DNA, 1131 bp, Solanum lycopersicum
  source 1..1131: mol_type = genomic DNA
atgtctgaaaatcaccctcatgttctcatctttccttatccagcacaaggacatatgcttccccttttggatttcactcatcagctagtcaacaatggagtatacatcactattttagtcacccccaaaaatctccccttcttgaacccccttctctccaggaacccatcaatcaaaactctcgttttgccttttccatctcacccttcaattcccgccggcgtagagaacgtcaaggaccttcctgctaatgggttcctttccatgatgtgcaatctcggaaaacttcgagatcccatattggaatggttccacaaccatccttcgccgccgtcagctattatttctgatatgtttttggggtttactcacgaaatcgccacccagctcggtattcgccgggtttggtctgttggaccagtacttcctccgggagaagaagatgatgtttccgtccaatcaaacagaggcggttcgagctctgttttagccagtgaaatactagcatggctcgacaaatgtgaagatcactcagttgtatacgtctgtttcggcagtcaagctgtattaacaaacaaacaaatggaggaactagccattgctttggacaaaagtggtgtccatttcattttgtcagccaaaagagccaccaaaggacacgcttcaaatgattacggtgtgattccgtcttggtttgaggaaaaagtggctggacggggcctggttgttagagactgggccccacaagtgttgattttaaaacaccgagctatagccacattcttaactcactgtggttggaattcgactctcgaaagcttgacagcaggaataccattgttaacatggccgatgggtgctgaccaattcgccaatgcaaaccttttagttgacgagcatgaggtagctattagagcctgtgaaggtgtgcaaacggtaccaaattcagacgagttggctgcgctactggcggaagctgtgcaaggaaacaaggtagaagaccggagacttcgtgcatcgaagctacgaaaaattgcaattaatggcattaaagaaggcgggaactcatttaaagaattggcagcgttcgtaaaacatttaagggaggaggctacaataatcgaagcgtaa

SEQ ID NO: 40 - DNA, 1371 bp, Solanum lycopersicum
  source 1..1371: mol_type = genomic DNA
atgactactcacaaagctcattgcttgatcttgccatatccagtccaaggtcatatcaacccaatgcttcaattctccaaacgtttacgatccaaacgcgttaaaatcactatagcactcacaaaatcctttttgaaaaacatgaaggaattgccaacttctatgtcaattgaggccatatctgatggctatgatgatggtggtcgcgatcaagcaggaactttcgtggcctatattacacgattcaaagaaattggttcggatactctgtctcaacttattcaaaaattggcaattagtggatgtcctgtaaattgcatagtatatgatccattcctcccttgggctgttgaagttgcaaaacaatttggattgattagtgctgcattttttacacaaaattgtgtagtggataacatttattaccatgttcataaaggggtaataaaacttccccctactcaaaatgatgaaaaaatcataattcctggattttcaagtccaattaaggcatcagatgtacctaattttgttattaatcctgaaggagaaagaatacttgaaatgttggtgaatcaattctcaaatcttgacaaagtagattgggtcctaatcaatagcttttatgagttggagaaagaggtaattgattggatgtcgaagatatatccaataaagacaattggaccaacaataccatcaatgtacttagacaagagactacatgatgataaagagtatggtcttagtatgttcaagccaatgacaaatgaatgcctaaattggttaaaccatcaaccaattagctcagtgttgtatgtatcatttggaagtttagccaaactaggaagtgagcaaatggaagaattggcatggggtttgaagaatagcaacaagagcttcttgtgggttgttaggtctactgaagagcccaaactccccaacaactttattgaggaattaacaagtgaaaaaggcttagtggtgtcatggtgtccacaattacaagtgttggaacatgagtcgatagggtgttttctgacgcactgtggatggaattcgacactggaagcgattagtttgggagtgccaatggtggcaatgccacaatggtcagatcaaccaacaaatgcaaagcttgtgaaagatgtttgggaaataggtgttagagccaaacaagatgaaaaagggatagttagaagagaagttatagaagaatgtataaaactagtgatggaagaagataaaggaaaattaattagagaaaatgcaaagaaatggaaggaaatagctagaaatgttgtggatgaaggaggaagttcagataaaaatattgaagagtttgtttccaagttggttactatttcctaa

SEQ ID NO: 41 - DNA, 933 bp, Solanum lycopersicum
  source 1..933: mol_type = genomic DNA
atggcttcttccaaatttccaagcataattgaaaccctaaaacctaacttgattatatatgatgggttccaaccatgggtagcaactatggcttcatcatacagtattcatgctattatgttttatgtttcttcaacttctggtcttgcctacatttaccaccaatttcttcatgggagttcaagccttacatcttttccattttcttccatataccttcatgaccatgagatcaagaaattaggcatacaaccaataaaaccacgcgatgagaaagcttttgcatacataatccttgagtcttttgaacaatctcacaacattgttttgttgaacacttgtagggagactgaggggaagtatatagattatgtttctacaataggaaagaaagagttgataccaattggaccattaattcgcgaggcgatgatagatgaggaggaggattgggggacaattcaatcttggctagacaagaaggatcaattatcatgtgtttatgtatcatttggaagtgaaagcttcttgtcaaagcaagaaattgaagagatagcaaaagggcttgagctaagcaaagttaattttatttggacaatcaaatttcctaaaggggtgaacaaaacaattgaagaaatggttccacaagaaagcatgagttttggcataccattaatagccatgcctatgaatcatgatcaaccattgaattcaagattagtggaggaacttggcataggggtggagattttgagaggtgaaaatggagaaataatgaaagaagaggtggcaaaaggaataaagaaagtggtagaggataacactaggaaacaagttaatttgaaggcaatggaattgagtgaaaagataaaattcaaaggggagaaagctattgatgaaggagttaaaaaagggttgaagcttttgtgttga

SEQ ID NO: 42 - DNA, 1413 bp, Solanum lycopersicum
  source 1..1413: mol_type = genomic DNA
atggtgcaaccccatgtcctattggtgacatttcctgcacaaggtcatatcaatccatctcttcaatttgccaagaggctaatcgaaatgggcattgaggtgacattcaccactagtgttttcgcccaccgtcgtatggcgaaaatcgcggcttccacggcacccaagggcttaaacttggcggcattctctgatggatttgatgatggtttcaagtctaacgtggatgattctaaacgttacatgtcagagataagaagtcgtgggtcccaaacattgagggatgtcattttgaagagttcagacgaaggacgtcctgtcacgtccctcgtctacactcttctgctaccttgggcagctgaggtagcgcgtgaactccatatcccatccgcgttactatggattcaaccagcaactgtgctagacatatactactattacttcaatggctatgaggatgaaatgaagtgtagtagctcaaatgatccaaattggagtatccaattgccaaggcttccattactaaaaagccaagatcttccatcttttttagtttcatctagctcaaaagatgataagtatagttttgctctaccaacattcaaagagcaactagacacattagatggtgaagaaaatccaaaggtacttgtgaatacatttgatgcattagagctagagccactcaaagctattgaaaaatacaatttaattggaattggaccattgattccttcatcattcttgggtggaaaagattcattggaatcttcatttggtggtgatctttttcaaaagtcaaatgatgactacatggaatggttaaacacaaagcctaaatcatcaattgtttatatctcatttgggagtctattgaatttatcaagaaaccaaaaggaggagattgcaaaagggttgatagagatccaaaggccattcttatgggtaataagagatcaagaagaagaaaaagaagaagagaaattaagttgcatgatggaattagagaagcaagggaaaatagtaccatggtgttcacaacttgaagtcctgacacatccatctttaggatgttttgtctcgcactgtggatggaattcgactctagaaagcctatcgtcaggtgtacctgtggtggcatttcctcattggacagatcaagggactaatgcaaagttgattgaagatgtttggaagacaggtgtcaggatgagagtgaatgaagatggtgtggttgagagtgatgaaataaaaaggtgcatagaaattgtaatggatggtggagaaaaaggagaagaaatgagaaaaaatgcacaaaaatggaaagaattggcaagagcagctgtgaaagaaggtggatcttcagaagtgaatctaaaagcttttgttctacaagttagcaaaagttgttga

SEQ ID NO: 43 - DNA, 1428 bp, Solanum lycopersicum
  source 1..1428: mol_type = genomic DNA
atgaaaatagaaagaaaacagagtgtagtactggtgccacacccataccaggggcatttaacaccaatgctacagcttggtagtatccttcattcacaaggcttttctgttatagttgcacatactcaatacaatactcctaattattccaatcatccacaattcgtcttccattctatggatgacggattacagggaatcgacatgtcattcccgagtttagaaaacatatatgatatgaacgaaaactgcaaggcgcctctcagaaactaccttgttagtatgatggaagaggaaggtgatcagcttgcttgtatcgtttatgacaacgtcatgttctttgtcgatgatgtagcgactcagttgaagcttcccagcattgtcctgcgcactttcagcgctgcgtatttgcactctatgatcaccattttacagcaacctgaaatatatttaccttttgaagattctcagctgctggatccgttaccggagcttcatcccttgagatttaaggatgtaccgtttcctatcatcaataatacagttccagaaccgatactagacttctgtagagcaatgagtgatataggatcatctgtcgcgactatatggaacacgatgcaagacttggagagttcaatgttgttacgccttcaagaacattacaaggtgcccttttttccaataggcccggtacacaaaatggcatctttggtctcatcgactagcatactagaagaagacaatagctgcatcgagtggctcgatagacaagcccctaactctgttctctatgtcagcttgggtagcctagtgaggattgatcacaaagagttgattgagactgcttggggattagctaatagcgatcaaccgttcttgtgggttattcgacctggctctgtctctggctttcaatgtgctgaggcactgcctgatggttttgagaaaatggtaggagaaagaggacgaatagtgaaatgggcaccacaaaaacaggtgcttgcacatcccgcggtagcagggtttttcactcattgtggttggaattctacgcttgaaagtatatgtgaagaagtccctatggtgtgcaggccatttctagcagaccaactggtgaacgcaaggtatctgagccaaatatacaaggttgggttcgaattggaggttatcgagagaacggtcatagagaaaacaataagaaaactcatgttaagtgaagaaggcaaagatgtgaagaaaagagtagcagacatgaaacaaaagatagttgctggaatgcagattgattgcacttcacataagaatctgaatgatttggtagacttcatttctgccttgccatcacgactcgctccgccaacgcctgtctttggggcaatcatgagctcaaaccacatagcaagcaagtgtatcatagagtcttga

SEQ ID NO: 44 - DNA, 1476 bp, Solanum lycopersicum
  source 1..1476: mol_type = genomic DNA
atgggtgttcttactatagagccacattttgtgttgtttcctttcatggcacaaggccatacaatccctatgattgatattgcgcgattactatcacaacgcggagttattatcacaatcgttacaacacatttgaacgcgaatagattcaagaaagtcattgatcgcgctatagaatcaggactgaaaattcaggtggttcatctctactttccaagcttagaggctggactgcctgaaggatgtgaaaatttcgacatgcttccatcgatggatttggggctgaaattcttcgatgctacggaaagacttcagccccaagtggaagaaatgttgcgagaaatgaaaccttcaccaagttgtataatatctgatatgtgtttcccatggacaactaatgttgcacaaaagttcaacattcctagaattgttttccatggaatgggttgcttttctttgttatgtttacataatttgaaagattgggaagggttagaaaagatcgagtctgatacagagtattttcgagtgcctggattgttcgacaagattgaactaaccaaaaatcagcttggaaatgctgccaggccgagaaatgaagagtggcgagtaatgagtgaaaaaatgaagaaagcggaggaggaagcttatggaatggtggtgaatacctttgaggatttggaaaaagaatacatcgagggtttaatgaatgcgaaaaacaagaaaatttggaccattggccctgtttcactctgtaacaaagaaaaacaggacaaagctgaaagagggaacgaggctgcaattgatgaacacaagtgtctaaactggcttgattcgtgggaacaaaactctgtactctttgtatgtctcgggagcctatcgcgcctttccacgtctcagatggttgagctagggcttgggttagaatcatcgagacgaccctttatttgggttgttagacacatgtcagatgagttcaagaattggctagttgaagaagattttgaggaaagagttaaagggcaaggactattaatccgcggttgggcgccacaagtactactcttgtcacatccttcaataggtgcgttcttgactcactgtggatggaattcgagcctggaaggtataaccgctggcgtggccatgatcacttggccaatgtttgctgagcagttttgtaatgagaggttaatagtggatgtactcaagacaggagtgaggtcaggcatagagaggcaagtgatgtttggagaagaagaaaaattgggaacacaagtaagcaaagatgacattaagaaggtgattgaacaagtgatggatgaagaaatggaaggtgaaatgagaagaaaaagagcaaaagaattaggagaaaaggcaaagagggctatggaggaagagggttcatctcatttcaacttgacacaattgattcaagatgtgacagagcaagcaaaaattttaaaacctatgtag

SEQ ID NO: 45 - protein, 412 aa, Solanum lycopersicum
MSVALLWVVSPCDVSNGTSFMESVREGNRFFDSSRHRNLVSNERINRGGGKQTNNGRKFSVRSAILATPSGERTMTSEQMVYDVVLRQAALVKRQLRSTNELEVKPDIPIPGNLGLLSEAYDRCGEVCAEYAKTFNLGTMLMTPERRRAIWAIYVWCRRTDELVDGPNASYITPAALDRWENRLEDVFNGRPFDMLDGALSDTVSNFPVDIQPFRDMIEGMRMDLRKSRYKNFDELYLYCYYVAGTVGLMSVPIMGIAPESKATTESVYNAALALGIANQLTNILRDVGEDARRGRVYLPQDELAQAGLSDEDIFAGRVTDKWRIFMKKQIHRARKFFDEAEKGVTELSSASRFPVWASLVLYRKILDEIEANDYNNFTKRAYVSKSKKLIALPIAYAKSLVPPTKTASLQR

SEQ ID NO: 46 - protein, 314 aa, Solanum lycopersicum
MAAGISASASSRTIRLRHNPFLSPKSASTAPPVLFFSPLTRNFGAILLSRRKPRLAVCFVLENEKLNSTIESESEVIEDRIQVEINEEKSLAASWLAEKLARKKSERFTYLVAAVMSSLGITSMAILAVYYRFSWQMEGGEVPFSEMLATFTLSFGAAVGMEYWARWAHRALWHASLWHMHESHHRPREGPFEMNDVFAITNAVPAIALLSYGFFHKGIVPGLCFGAGLGITVFGMAYMFVHDGLVHKRFPVGPIANVPYFRRVAAAHQLHHSDKFDGVPYGLFLGPKELEEVGGLEELEKEVNRRIKISKGLL

SEQ ID NO: 47 - protein, 765 aa, Solanum lycopersicum
MWKLKIAKGQDDRYLYSTNNYIGRQIWEFDPNAGTIEEQAKIEEARQHYWNNRYKVKPNSDLLWRMQFLREKNFKQRIGAVKVEEGEEISHEIATIALRRAVHFFSALQATDGHWPAENAGLLFFLPPLVMCMYITGHLNTVFPAEHRKEILRYIYCHQNEDGGWGLHIEGHSTMFCTALSYICMRILGEGPDGGERNACARARKWILDHGSVTAIPSSGKIWLSILGAFEWLGTNPIPPEFYILPSFLPMHPAKMWCYSRIFYMPLSYLYGKRFVGPITPLILQLREELYDQPYNEINWKRVRHLCAKEDLYYPHPLVQDLMWDTLYFFYEPLLTRWPFNKLRNKALEVTMKHLHYEDENSRYITVQCVNKVLCMLACWVEDPNGSYFKKHLARVPDYLWVAEDGMKMQNSGSQSWDTSFAIQALLASEMNDEISDTLRKGHDFIKKSQVKDNPSGDFRGMYRHISKGSWTFSDQDYGWQSSDTTAEALRCCLFFSTMPSKLVGEAMEPARLYDSVNVILSLQSKNGGLAAWEPAGTSKYLELLNPTEIFEDVVIEHEYVECTGSSIQALVHFKKLYPGHRTTEIDNFIDNAVKYIENVQRPDGSWYGFWGVCFTYASWFALGGLAAAGKSYSNSAAVRKGVDFLLQRQRSDGGWGESYHSCPDKVYRELETEHSNLVQTAWALMGLIHSGQADRDPKPLHRAARLLINSQMEDGDFPQQEITGAFMKNCMLHYATFRNIFPLWGLAEYRKNVSPTKLEEDIKK

SEQ ID NO: 48 - protein, 612 aa, Solanum lycopersicum
MWKLKIAKGQDDRYLYSTNNYIGRQIWEFDPNAGTIEEQAKIEEARQHYWNNRYKVKPNSDLLWRMQFLREKNFKQRIRAVKVEEGEEISHEIATVALRRAVHFFSALQATDGHWPAESAGPLFFLPPLVMCMYITGHLNTVFPAEHRKEILRYIYCHQNEDGGWGLHIEGHSTMFCTAMSYICMRILGEGPEGGVNNACARARKWILDHGSVIAIPSWGKTWLSILGAFEWIGTNPMPPEFWILPSFLPVHPAKMWCYCRTVYMPMSYLYGKRFVGPITPLILKLREELYDQTYDEINWKKVRHVCAKEDLYYPHPFVQDLMWDSLYICTEPLLTRWPFNKLRNKALEVTMKHIHYEDENSRYITMGCVEKVLSMLACWVEDPNGDHFKKHLARIPDFLWVAEDGMKMQGCGSQSWDASLAIQALLASEMNDEISDTLKNGHDFIKQSQVKDNPSGDFKVMYRHISKGSWAFADQDLGWQVSDCTAEALKCCLLFSTMPPEIVGEAMDPVRLYDSVNVILSLQSKNGGLSAWEPAGAPEYLELLNPTEFFEDIVIEHEHVECTSSAIQALVRFKKLYPGHRTTEVDNFINNGVKYIEDVQEPDGSWYMIFL

SEQ ID NO: 49 - protein, 517 aa, Solanum lycopersicum
MEIWFIVVVTLCVAFWLKSICNIIVSPNSNTKRNLPPGPYSFPVIGSLLWAKRTFSDLEPILRDLKAKYGPLITLNIGSRSAIFVASHSLAYQALVQQGAIFSNRPKAGPTSAVVNSNQRNISSAPYGPIWRLLRRNLTSEILHPSRIKSYSKARSWVLGILIQQLRNAQVDSVKLIDHFQYAMFCLLVLMCFGDKLEETQIRQIENIQRKLLLGFTRFNIINIFPRVGKIIFRNRWKELIELRQEQESIIIPLIEARSRAREQRTEHGDEFVVAYVDTLLNLEFPEEKRNLNHGEIVTLCSEFLSAGTDTTSTALQWIMANLVKNPSIQEKLYQEIATVVGEKQSKLTDEEVVKEEDLQKMPYLKAVILEGLRRHPPGHFVLPHTVTEEVELNGYVIPKDVTINFMVAEMGLDPKVWEDPLEFRPERFLVEGSDNEGFDITGSREIKMMPFGAGRRICPAYALAMLHLEFFVANLIWHFQWKPVEGDDVDLTEILEFTVVMKNPLRARICPRVNSV

SEQ ID NO: 50 - protein, 506 aa, Solanum lycopersicum
MEFPWYSVVVPLIVFLFFLHNCLFTIFNTSNKKLPPSPRKLPIIGNLYQLGLHPHRSLYKLSKKYGPVMLLHLGSKPVLVASSVDAARDILKTHDLVWSTRPKSSIADGLLYGSKGVAFSNYSEYWRQVRSVIVLHLLSNKRVQSFRDIREEEVSNMIDEIRKRSTSSSNSVIDMRDVLSCMTNNIINRVTIGRTYNEGESGRAVKALLEDLLALLGTFNIGEYIPWLKWLNKINGLDNRVKKVAKDLDAFLDSVIEERVVRNKKAENSTGEAKDFVDVLLEIQNGNETGFPLQRDSLKAILLDSFIAGVDSIYTTLEWIMIELLRNPRAMEKLQNEVRGLVQGKAEITEDDLGNMLYLKAVIKESLRLNPPFPIPVPRESMEDVKLLDYNIPAKTQVLVNIWAIGRDPLSWDEPEEYRPERFLNSDIDFRGLNFELIPFGAGRRGCPGIPFAIVIIELTLARLVNKFNFALPQGIKKEDLDMSECTGISIRRKLPLLAVATPCSV

SEQ ID NO: 51 - protein, 519 aa, Solanum lycopersicum
MLYTTPSNLTRRVWFNIESIFSSYSMTLIWTILVVGLSVYALYELLNIHKRKRYPPGPTGLPILGHLHLLGKNPHKDLQNLAQKHGPIMYMRLGLVPTIIASSADAAEKVLKTYDHIFASRPHHEASQHLAYGQKNLVFAKYDVYWRNIRKLCTVHLLSNQKIHSFQSMRKQEVELLIESLKQEARDRVVIDLSAKVTSLNANLTCLMVFGKKYMDEDLDKRGFKAIVQDVVHLAGMPNLGDFFPFLGAIDLQGITRKLKDLSKVFDEFLEKIIDEHVYAHEHKQNKDFVDTMMDIMQSGDAKFQFDRHHIKAILFDMLIAAMDTTASSTEWILTELLRHPQVMKELQKELQEVVGLDRMVEESDLENLKYLDMVVKEGLRLHPVAPLFYHESIEDCVVDGFHIQKGTRIIINCYAIHMDPNVWPDPEKFLPERFVGSSVDFRGRDFQLVPFGSGRRSCPGMQLAVMVVRLVVAQLVHCFEWELPNGIESCDLDIDEKFGIVTCREKPLLAIPTYRLNK

SEQ ID NO: 52 - protein, 477 aa, Solanum lycopersicum
MDLIIFIVMLLVMVLVVVLLVLLRTNKASPKEMEAIPGTLGWPIIGESLSFISEFSSPAGIYSFINKRQKLYGKVFKSYVLGRYTVFMTGREASKILLTGKDGIVSLNLFYTGQQVLGPTSLLQQTGEAHKRLRRLIAEPLSVDGLKKYFQFINSLAIETLDQWSGRKILVLEETSTFTLKVIGNMIMSLEPTGEEQEKFRTNFKIISGSFASLPFKVPGTAFYRGIQARDRMYAMLDSIIDQRRSGENIKQDFLQSLVKKHGKDAPEGDDDDKLTDKQLKDNILTLLVAGHDTTTAALTWLLKFLQENPAVLERLREEHREIQAQKQGTLDLTWSEVNNMPYTAKVISETLRMATILPWFSRKAAQDFEIEGCKIKKGWSLNLDVVSIHRDPKIFPNPEKFDPSRFDDPLKPFSFLGFGSGPRMCPGINLAKLEISVFLHHLVCRYKWTPLDTDDSVQPTLVRMLKNKYPVMVEPL

SEQ ID NO: 53 - protein, 517 aa, Solanum lycopersicum
MEILYNTIIATICVAILLVYTWRVLNWAWFRPKKLENFLRQRGLKGNPYKLLYGDLNELTKSIVEAKSKSINISDDITQRLIPFFLDSINKNGKSSFMWLGPYPTVLITNPEHVKEILTKNYVYLKQTHPNPFAKLLAQGLVLVEEDKWAKHRKIINPAFHVEKLKHMLPAFYMSCSEMISKWEDIVSKETSYELDVWPDLQIMTSEVISRTAFGSSYEEGRIVFELQQEQAEHIMDISRSIYIPGSRFLPTKRNKRMLEIEKQIQTTIRHIIDKRLRAMEAGETSKDDLLGILLESNMKEIEQHGNKDFGLTTTEVIEECKLFYFAGQETTSVLLVWTMVLLCLHPEWQVRAREEVLQVFGNEKPDLEGLSHLKIVTMILYETLRLFPPLPVFSRRNKEEVKLGELQLPAEVILIIPAIFIHYDKEIWGEDAKEFKPERFSEGVSKATKGQVSFIPFGWGPRICIGQNFAMMEAKMAIAMILQKFSFELSPSYTHAPFATITIHPQYGAPLLLRKH

SEQ ID NO: 54 - protein, 514 aa, Solanum lycopersicum
MIVTIFCCAIAITLFVCLWKVLNWVWFQPKKLEKLLRKQGLNGNSYRFLYGDMKDFSKMIKEANSKPMNMDDHDLAPRMVPFFLETIKKYGKKCFTWMGPRPQILIMDPELIKEVLSKTYVYQKPHGNPLGTLFVQGLVSYEKDKWAKHRKIINPAFHLEKLKHMLPAFYLSCSEMLSKWEDVVRVEGSHEIDVWPHLQQLTCDVISRTAFGSSYEEGRKIFELQKEQAQHFLEVIRSVYIPGWRFLPTKRNRRMKELKNDVRSSIRGIIDKRLKAMEAGNADNEDLLGILLESNFKEIELHGNKDFGMSIEEVIEECKLFYFAGAETTSVWILWTLVLLSRHPDWQERAREEVLQVLGSRKPDFDGLNHLKVVTMILYESLRLYSPITVLTRRVYEDITLGEVSLPTGVLVSLPMILLHHDKDIWGEDATKFNPERFSEGISSATKGQVTYFPFAWGPRICIGQNFAILEAKMTLCMILQSFSFELSPSYTHAPQSLVTTQPQYGAPLIFHKL

SEQ ID NO: 55 - protein, 519 aa, Solanum lycopersicum
MDEIQILVRVCCSAIAIALFVCLWKVLNWVWLNPKKLENLLRKQGLNGNSYKILYGDLNDFIGMIREASSKPMNLSDDIAPRLVPFFLETTKKYGKKCFIWLGPKPQVIIMDPELIKEVLSKTYLYQKPGGNPFAALLVQGLATYEEDKWAKHRKIINPAFHLEKLKHMLPAFHLSCTEMLSKWEDAVPLGSSREIDVWPHLQQLTCDVISRTAFGSSYEEGRKIFELQTEQAQNFIDAVREVYIPGRRFLPTKRNRRMKEIKNEVRTSVKGIIDKRMKAMKAGNADNNEDLLGILLESNFKEIEQHGNKDFGMSIEEVIEECKLFYFAGQETTSVLLVWTLVLLSRYQDWQTQAREEVLQVFESRKPDFDGLNHLKVVTMILYESLRLYPPLITLNRQVNEDIVLGELCLPAGVLVSLPMILLHHDKDIWGEDANEFNPERFREGISSATKGKVTYFPFSWGPRICIGQNFAMLEAKMALCMILQSFSFELSPSYTHAPKSLVTMQPQYGAPLILHKL

SEQ ID NO: 56 - protein, 476 aa, Solanum lycopersicum
MMIIDLEFSTLLICLVPFLFIFFINLFHSKSTKIPKSYPIIGSYFSILANKHRRIQWSSDIIQTTTNLTFTLIRPFGHMFKSTMADFLGDGIFNVDGEIWKYQRQVASHEFNTKSLRKFIETVVDAELNERLIPLLAVAASEKKVVDLQDILQRFAFDNICKIAFGYDPAYLSPSLPQEKFAVAFEEAVKLSSERFNAIFPFLWKIKRHFDFGSEKRFRIAVDEVRQFAKQLVREKQSELNQKSSLDSVDLLSRFLSVGHRDDDFVTDVVISFILAGRDTTSAALTWFFWLISEHPVVESSILNEIKLKSHTPVYEEVKEMIYTHASLCESMRFYPPVPIDSKTATEDNVLPDGTFVKKGTRVSYHPYAMGRAEELWGSDWNKFKPERWLDKDEVTGNWNFVTKDMYTYPVFQAGPRICLGKEMAFLQMKRVVAGVLRRFKVVPVTEKGVEPVFISYLTAKMKGGFHVTIEERDHE

SEQ ID NO: 57 - protein, 503 aa, Solanum lycopersicum
MNHLDVFWWLKSFHVYSILIFIIISLFSLIFYMVRVKLWCNCEICHAYITTSWSSQFINLCDWYTHLLKESPTRTIHIHVLGNIITSNTQNVEYMLKTRFDNYPKGKTFSTILGDFLGRGIFNVDGDLWRFQKRMSSLELGKVSIRSYAFEVVKNEIDKRLVPLLHEYKQGGGVLDFQDVFRRFSFDCICRFSFGLDPKCLESSLPMSQFALSFDLASKLSAKRAMTTSPIVWKIKRFFNIGSEKELREAIKVINILAQEVIRQKRKLAFSNHRDLLSRFMGSISDETYLRDIVISFLLAGRDTIASALTSFFYVIGNNPQVAKAIREEANRVLGPNEDLTSYEQMSELHYLQACVYESMRLYPPIQFDSKFCLEDDFLLDGTFVKKGTRVTYHPYAMGRMEELWGCDALEFKPQRWLKDGVFVQENPFKYPVFQAGLRVCLGKEMALVEVKSVALSLLRRFHVELAQPYHHNPRFSPGLTASFKAYSTNPNHGGWNSFQHIN

SEQ ID NO: 58 - protein, 470 aa, Solanum lycopersicum
MGQLHFFFFPMMAQGHMIPTLDMAKLVACRGVKATIITTPLNESVFSKAIERNKHLGIEIDIRLLKFPAKENDLPEDCERLDLVPSDDKLPNFLKAAAMMKDEFEELIGECRPDCLVSDMFLPWTTDSAAKFSIPRIVFHGTSYFALCVGDSIRRNKPFKNVSSDTETFVVPDLPHEIRLTRTQLSPFEQSDEETGMAPMIKAVRESDAKSYGVIFNSFYELESDYVEHYTKVVGRKNWAIGPLSLCNRDIEDKAERGRKSSIDEHACLKWLDSKKSSSIVYVCFGSTADFTTAQMQELAMGLEASGQDFIWVIRTGNEDWLPEGFEERTKEKGLIIRGWAPQVLILDHEAIGAFVTHCGWNSTLEGISAGVPMVTWPVFAEQFFNEKLVTEVMRSGAGVGSKQWKRTASEGVKREAIAKAIKRVMASEETEGFRSRAKEYKEMAREAIEEGGSSYNGWATLIQDITSYR

SEQ ID NO: 59 - protein, 466 aa, Solanum lycopersicum
MADDQLHIFFFPLMAHGHIIPTLDMAKLFVSHGAKATIITTPLNLPIFVQSSEKIKHLGLEIYVKAIRFPAVEAGLPEGCERMDQLTSDDLVPKFFKATTMLQQQLELLLHECQPNALVADMFFPWATESAAKFDIPRLVFHGMGFFALCALENLRHYKPQKNVCSDTEPFVVPNLPHKVKLTRMKLAPYDREETEMTKFIEQVKGSEFTSFGVIVNSFYELEPDYADYYRKVLGKRSWHVGPFLLCNRKNEEKIHKSDDKHHECLQWLDTKKQNSVIYVCFGSMSNFTDSQIEEIALGLEASEQEFIWVIRKGKIPEGFEERTKEKGFIIRGWAPQTLILDHEAIGAFLTHCGWNSTLEGVSAGVPMVTWPIFAEQFLNEQLITKVLQIGVAVGAEEWSIVVDNVKSEAITKAVKCVMAGEESMEMRRRAEEVKKMANKAVEEGGSSYSDLNALFQELRTINLHN

SEQ ID NO: 60 - protein, 479 aa, Solanum lycopersicum
MENEKDKLHVVMFPWLAMGHIIPFFHLSKCLARRGHRVTFISTPRNIERVVNKVPSDLAHLLHIVCFHLPKVENLPDDAESSMDIPYQKAQFLKIAFDSLEIPLTNYLESSSNPKPDWIVYDYASHWLPQIAGKLGVSRAFFSLFTAATMSFYGPPCALLNDERSIAEDYTVVPKWIPFETKVVYRLHEIKKNFEAPADESGTSDGARFGASIDASDVILFRNCVEFESEWFSLVSELYQKPIISIGVLPPSVVVDHEQDDSNDASWSGIKNWLDKHNQDSVVYVALGTEATLNQQELNELALGLEKCGLPFVWVLRDQPKHNQQDSCIQLPDGYEDRVKNRGVIYKGWVPQTKILSHSSVGGFLTHCGWNSVIEALCFGRVLVMFPVLNDQGLNTRLLQEKGVGVEIPRNEKDGFFTSDSVAEAVKFGVVSEEGELLRANARQMSCLFGDRKRNEQLIDDCVGYFMENRISKSNSSIG

SEQ ID NO: 61 - protein, 376 aa, Solanum lycopersicum
MSENHPHVLIFPYPAQGHMLPLLDFTHQLVNNGVYITILVTPKNLPFLNPLLSRNPSIKTLVLPFPSHPSIPAGVENVKDLPANGFLSMMCNLGKLRDPILEWFHNHPSPPSAIISDMFLGFTHEIATQLGIRRVWSVGPVLPPGEEDDVSVQSNRGGSSSVLASEILAWLDKCEDHSVVYVCFGSQAVLTNKQMEELAIALDKSGVHFILSAKRATKGHASNDYGVIPSWFEEKVAGRGLVVRDWAPQVLILKHRAIATFLTHCGWNSTLESLTAGIPLLTWPMGADQFANANLLVDEHEVAIRACEGVQTVPNSDELAALLAEAVQGNKVEDRRLRASKLRKIAINGIKEGGNSFKELAAFVKHLREEATIIEA

SEQ ID NO: 62 - protein, 456 aa, Solanum lycopersicum
MTTHKAHCLILPYPVQGHINPMLQFSKRLRSKRVKITIALTKSFLKNMKELPTSMSIEAISDGYDDGGRDQAGTFVAYITRFKEIGSDTLSQLIQKLAISGCPVNCIVYDPFLPWAVEVAKQFGLISAAFFTQNCVVDNIYYHVHKGVIKLPPTQNDEKIIIPGFSSPIKASDVPNFVINPEGERILEMLVNQFSNLDKVDWVLINSFYELEKEVIDWMSKIYPIKTIGPTIPSMYLDKRLHDDKEYGLSMFKPMTNECLNWLNHQPISSVLYVSFGSLAKLGSEQMEELAWGLKNSNKSFLWVVRSTEEPKLPNNFIEELTSEKGLVVSWCPQLQVLEHESIGCFLTHCGWNSTLEAISLGVPMVAMPQWSDQPTNAKLVKDVWEIGVRAKQDEKGIVRREVIEECIKLVMEEDKGKLIRENAKKWKEIARNVVDEGGSSDKNIEEFVSKLVTIS

SEQ ID NO: 63 - protein, 310 aa, Solanum lycopersicum
MASSKFPSIIETLKPNLIIYDGFQPWVATMASSYSIHAIMFYVSSTSGLAYIYHQFLHGSSSLTSFPFSSIYLHDHEIKKLGIQPIKPRDEKAFAYIILESFEQSHNIVLLNTCRETEGKYIDYVSTIGKKELIPIGPLIREAMIDEEEDWGTIQSWLDKKDQLSCVYVSFGSESFLSKQEIEEIAKGLELSKVNFIWTIKFPKGVNKTIEEMVPQESMSFGIPLIAMPMNHDQPLNSRLVEELGIGVEILRGENGEIMKEEVAKGIKKVVEDNTRKQVNLKAMELSEKIKFKGEKAIDEGVKKGLKLLC

SEQ ID NO: 64 - protein, 470 aa, Solanum lycopersicum
MVQPHVLLVTFPAQGHINPSLQFAKRLIEMGIEVTFTTSVFAHRRMAKIAASTAPKGLNLAAFSDGFDDGFKSNVDDSKRYMSEIRSRGSQTLRDVILKSSDEGRPVTSLVYTLLLPWAAEVARELHIPSALLWIQPATVLDIYYYYFNGYEDEMKCSSSNDPNWSIQLPRLPLLKSQDLPSFLVSSSSKDDKYSFALPTFKEQLDTLDGEENPKVLVNTFDALELEPLKAIEKYNLIGIGPLIPSSFLGGKDSLESSFGGDLFQKSNDDYMEWLNTKPKSSIVYISFGSLLNLSRNQKEEIAKGLIEIQRPFLWVIRDQEEEKEEEKLSCMMELEKQGKIVPWCSQLEVLTHPSLGCFVSHCGWNSTLESLSSGVPVVAFPHWTDQGTNAKLIEDVWKTGVRMRVNEDGVVESDEIKRCIEIVMDGGEKGEEMRKNAQKWKELARAAVKEGGSSEVNLKAFVLQVSKSC

SEQ ID NO: 65 - protein, 475 aa, Solanum lycopersicum
MKIERKQSVVLVPHPYQGHLTPMLQLGSILHSQGFSVIVAHTQYNTPNYSNHPQFVFHSMDDGLQGIDMSFPSLENIYDMNENCKAPLRNYLVSMMEEEGDQLACIVYDNVMFFVDDVATQLKLPSIVLRTFSAAYLHSMITILQQPEIYLPFEDSQLLDPLPELHPLRFKDVPFPIINNTVPEPILDFCRAMSDIGSSVATIWNTMQDLESSMLLRLQEHYKVPFFPIGPVHKMASLVSSTSILEEDNSCIEWLDRQAPNSVLYVSLGSLVRIDHKELIETAWGLANSDQPFLWVIRPGSVSGFQCAEALPDGFEKMVGERGRIVKWAPQKQVLAHPAVAGFFTHCGWNSTLESICEEVPMVCRPFLADQLVNARYLSQIYKVGFELEVIERTVIEKTIRKLMLSEEGKDVKKRVADMKQKIVAGMQIDCTSHKNLNDLVDFISALPSRLAPPTPVFGAIMSSNHIASKCIIES

SEQ ID NO: 66 - protein, 491 aa, Solanum lycopersicum
MGVLTIEPHFVLFPFMAQGHTIPMIDIARLLSQRGVIITIVTTHLNANRFKKVIDRAIESGLKIQVVHLYFPSLEAGLPEGCENFDMLPSMDLGLKFFDATERLQPQVEEMLREMKPSPSCIISDMCFPWTTNVAQKFNIPRIVFHGMGCFSLLCLHNLKDWEGLEKIESDTEYFRVPGLFDKIELTKNQLGNAARPRNEEWRVMSEKMKKAEEEAYGMVVNTFEDLEKEYIEGLMNAKNKKIWTIGPVSLCNKEKQDKAERGNEAAIDEHKCLNWLDSWEQNSVLFVCLGSLSRLSTSQMVELGLGLESSRRPFIWVVRHMSDEFKNWLVEEDFEERVKGQGLLIRGWAPQVLLLSHPSIGAFLTHCGWNSSLEGITAGVAMITWPMFAEQFCNERLIVDVLKTGVRSGIERQVMFGEEEKLGTQVSKDDIKKVIEQVMDEEMEGEMRRKRAKELGEKAKRAMEEEGSSHFNLTQLIQDVTEQAKILKPM

SEQ ID NO: 67 - DNA, 2322 bp, Plectranthus barbatus
  source 1..2322: mol_type = genomic DNA
atgaaaatgctgatgataaaatcacagttccgagttcattcaattgtctccgcatgggcaaacaactccaacaagcgtcaatcacttgggcatcagattcgtagaaagcagcgttcccaggtcacagaatgcagagtagcatcactggatgcattaaatggtatacagaaagtcggcccagcaaccattggtactcctgaagaggaaaataaaaaaatagaggactcaatcgaatacgtgaaagagttactcaaaaccatgggcgacggtaggatatccgtgtctccttatgatacagccatcgtagctctcatcaaagacctagaggggggtgatggtccagaatttccaagttgtctcgagtggattgcccagaatcaactcgcagacggctcatggggggaccacttcttctgtatatatgacagagtcgttaacacagccgcttgcgtggtcgctctaaaaagttggaatgtacatgccgataaaattgagaagggcgccgtttacctaaaggaaaatgtgcacaaattgaaggatgggaaaatcgagcatatgcctgccggttttgagttcgttgtccccgccacactagaaagagctaaggccctcggcataaaagggttgccatatgatgatccattcatccgagaaatttacagtgccaaacagaccagactcacaaaaatcccaaaaggaatgatatacgagagccctacttccctcctctactccttagatgggcttgaaggacttgaatgggacaaaatccttaagctacaaagcgctgacggaagctttataacatctgtcagctcaaccgccttcgtgttcatgcatactaatgatcttaaatgtcacgcatttataaaaaatgctttaactaactgtaacgggggcgttccacacacttatcctgtagacatcttcgcccgactttgggcagtcgatagactccagagactagggataagcaggttctttgagcccgagatcaagtatttgatggatcatatcaacaacgtgtggagggaaaaaggtgtgttctcaagtagacatagccagtttgccgatattgacgatacaagcatgggcattagactattaaagatgcacgggtacaatgtcaaccctaacgcacttgaacattttaaacagaaagacgggaagtttacttgttacgcagatcaacatatcgagtctccatctcccatgtacaacctataccgagcagcacaattacgatttcctggggaagaaatacttcaacaagccctacagttcgcttacaatttcctacacgaaaatttggcaagtaaccatttccaagagaagtgggtaatatccgatcacctaatagatgaagttaggataggacttaaaatgccatggtacgccaccctccctcgtgtcgaggctagctattacctgcagcactatggaggctcatccgacgtatggatcgggaaaaccctctatcgtatgccagaaatttctaacgacacttacaaaatccttgcacagctagattttaacaagtgccaggctcaacatcagctggaatggatgtccatgaaagaatggtatcagagcaacaacgttaaagagtttggaattagcaaaaaggagttacttcttgcctacttcttggctgctgctactatgttcgaacctgaaaggacccaagagcgaatcatgtgggccaagactcaagttgtctctcgaatgattacatccttcctcaacaaggaaaacaccatgagttttgatctcaagatagccctgctgactcaacctcagcaccagattaatggcagcgaaatgaaaaatggcctggcacagaccttgcctgcagccttccgacagttattgaaggaatttgataaatacactcgacatcaactccgtaacacatggaataagtggttgatgaagctcaagcagggcgacgacaacggtggtgccgatgcagagctactagcaaacaccttgaatatatgcgctggtcataacgaggacatattgtctcactatgagtatacagcattgagttcactcacaaataaaatctgtcagcgtttaagtcagatacaggacaagaaaatgctcgagatagaagaggggtctataaaggataaagagatggagctagaaatacagactttagttaagctggttctgcaagaaacatcagggggcatagacaggaacattaagcaaacatttctgagtgttttcaaaaccttttactaccgtgcctaccacgacgctaagactatagatgcccatatcttccaggtgctcttcgagcctgtggtgtag

SEQ ID NO: 68 - DNA, 1740 bp, Origanum majorana
  source 1..1740: mol_type = genomic DNA
atggcatcccttgcttttactcccggtgctgcaacattttcaggaaacgtggtcagaagaaggaaggacaatttcccagtacatggatttcccactacaattaggtctagcgtttctgttaccgtaaaatgttacgtttccacaaccaacttaatggttaaaatcaaagagaaatttaagggcaagaacgttaatagtttgactgtcgaagccgcagacgacgatatgccatcaaatctctgcattattgatacactgcaaaggctaggtattgatcgttattttcagccccaagttgacagcgtattagaccacgcttataaactttggcaagggaaggagaaagacaccgtatactcagatatatcaatacacgcaatggctttcagactactcagagtcaagggataccaagtttcctccgaagaactggacccctatatagatgtggaaagaatgaagaaacttaaaaccgtcgatgttcctactgttattgagttatatagagctgctcaagaacgaatgtatgaggaagagggatctctggagaggctgcacgtttggtccacaaacttcttaatgcatcaactacaagccaattcaatccccgatgaaaagctacacaaactggtggagtactacttaaaaaattatcatgggatacttgatcgtgtgggagtccgaaggaatctcgacctcttcgacatctctcattaccctaccttaagggccagagtccccaacttgtgtactgaggattttttgtctttcgctaaagaagatttcaatacctgtcaggctcaacaccagaaagaacatgaacagttacagcgttggttcgaagattgtaggtttgacacattaaaattcgggagagaaaccgctgtcggagcagcacattttctcagcagtgctattttaggcgaaagcgaattatgcaatgtcaggctcgccctcgcaaaacacatggtccttgttgtatttatagatgattttttcgatcattacgggagcagagaagatagttttaaaatcttgcaccttctaaaggaatggaaagaaaagcccgccggcgaatatggttctgaagaagtagaaatcctttttactgccgtatacaataccgtcaacgaattagccgagatggcccacgtcgagcaaggccgtaatataaaggggtttctaatcgaactgtgggtggagatcgtaagtatattcaagatcgaactggatacttggagtaacgacactacattaacccttgacgaatacttgtcaagcagttgggtatccgtcgggtgtagaatttgtattctggtttctatgcagctgttaggggtgcaactcactgatgagatgctattgtctgacgagtgtataaacttgtgcaagcatgtgtccatggtggataggcttttaaatgacgtaggaacattcgagaaggagcgaaaggaaaacactggcaacagcgttagtctgttgttagccgccgccgtaaaagagggccgacctataaccgaagaagaggccattatcaaaatcaagaaaatggctgaaaatgaacgacgtaaattgatgcaaattgtctataagagagagtcagtatttccccgtaagtgcaaagacatgttcctgaaggtgtgtcgtattggttgttacttgtatgcttcaggggacgagttcactagtccccagaaaatgaaggaggacgtgaagtcactcatttatgagtccttatga

SEQ ID NO: 69 - DNA, 2169 bp, Plectranthus barbatus
  source 1..2169: mol_type = genomic DNA
atggccagctgcggggccatcggttcttcatttttgcccttgctgcacagcgatgagagttccctgttatctcgacccactgccgcactgcatataaaaaagcagaagttcagcgtaggagcagcactctatcaagacaatactaatgatgtcgtcccaagtggggagggtctgacccgtcagaagcccagaaccctctcattcacaggagaaaaaccaagcacccctattttagacactataaactatcctatacacatgaaaaatctgtcagtcgaagagttggagatcttagctgacgaacttcgagaagaaatagtgtacacagtgagtaaaactggtggccacttatcctcatcattgggcgtaagtgagttgaccgtagcattgcaccacgtttttaatacccctgatgataaaatcatctgggatgtgggacaccaggcttatccacataaaatactcacaggtagaagaagcagaatgcatacaatccgtcagacttttggactggctgggtttcccaagagggacgaaagcccacacgacgctttcggggccggacacagttcaacctctatttctgcaggtttaggtatggctgtcgggagagatttgttacaaaaaaacaaccacgttatctcagttattggcgatggagccatgacagccggccaagcttacgaagctatgaacaatgcagggttcttagactccaacttgatcattgtactgaatgacaacaagcaagtgagtctgccaaccgcaacagtagatggtcctgcccctcccgtcggcgccctctccaaggcactaactaagctacaagcatcccgaaaatttaggcagctgcgagaggcagccaaaggtatgaccaaacaaatgggtaaccaagcccatgagattgcttctaaagtcgatacttacgtaaaggggatgatgggaaagcctggagcctcattattcgaagaacttggcatctactatatcgggcctgtcgacggccataatatcgaggacttggtatatatatttaagaaagtgaaagagatgccagcaccagggccagtcctcattcacatcatcaccgaaaagggcaagggctaccccccagcagaggttgctgccgacaaaatgcatggggtcgtaaagtttgatccaacaactggaaaacagatgaaggtaaagaccaagacccagtcatatacacaatatttcgccgaaagtctagtcgcagaggccgaacaagacgagaaagtagttgctatccacgccgccatggggggtggtactggcctcaatatattccagaaacgatttccagatcgatgttttgacgtcgggatagctgagcagcacgctgtaaccttcgccgcaggactagctaccgaagggcttaagccattctgtaccatctatagctcattcctccaaaggggctacgatcaggttgtacatgatgtggacctgcaaaagctacccgtgaggtttatgatggatcgagcagggttagtgggagccgacggacccacacattgtggggcttttgataccacctacatggcatgcctgccaaacatggtggtaatggcacctagtgacgaggcagagctcatgcatatggtggctaccgcagccgtgatagatgaccgaccatcctgtgtgaggtatccccgaggtaatggtattggagttccactaccacccaacaacaagggcatccctctagaagttggtaagggtcgaatactcaaggaaggaaatcgtgtagctattctcggttttggcactattgtacaaaactgtcttgccgctgctcaattactgcaggaacatggtattagcgtgagtgtggctgacgctaggttctgtaagccattggacggtgatctcattaaaaatctagtgaaggaacatgaggtactgatcaccgtagaggagggtagtataggtgggttttccgcccatgtttctcatttccttagcctgaatggactgcttgacggtaacttaaaatggaggcctatggtccttccagaccgatacattgaccatggcgcttacccagatcaaatagaggaggccggcctctcttccaagcacatagccggcactgtcctcagtttaatcgggggaggtaaagactctctacatttgattaacatgtaa

SEQ ID NO: 70 - DNA, 1083 bp, Plectranthus barbatus
  source 1..1083: mol_type = genomic DNA
atgaggagcatgaaccttgttgacgcttgggtacaaaacctacccattttcaagcagcctcatcctagcaaatttattcaccaccccaggttcgagccagcatttcttaagagtcgtcgaccaattagcagtttcgccgtttccgcagtgcttacaggagaggaagctcgtatcttcaccagaggagatgaggcccccttcaatttcaacgcatatgttgttgagaaggcaactcacgtaaacaaagctttagatgatgcagtcgcagtcaagaatccacccatgatccacgaagccatgcgatactctcttctagccgggggaaagagagtcaggcccatgctgtgcatagctgcttgcgaagtcgtagggggcccacaggctgctgctattcctgcagcctgcgccgtggaaatgatccacacaatgtcacttatccatgatgacttgccctgtatggataatgatgatttgagaaggggcaaacccaccaaccacaaggttttcggagaaaacgtagccgttcttgctggtgacgcattactcgcattcgcattcgagtttatcgccaccgctactacaggggtggcacctgaaaggatcttggccgccgtcgctgagcttgctaaagccatagggacagagggtctagtcgctggacaagtcgtagacttgcattgtactggaaacccaaacgtcggacttgataccctagaatttattcatatacataagaccgcagcattattggaggcaagcgtggtgctcggggccatcctcggcgggggaagttccgatcaggtagaaaagctacgaaccttcgcccgtaaaatcggattgctgttccaggtcgttgatgacatcctagacgtaaccaaaagtagtgaggaattgggtaagactgccggaaaggatctagcagtcgacaagacaacttaccctaagcttctaggattagagaaagctatggagtttgccgagaggttaaatgaagaggccaaacaacaattacttgattttgaccccagaaaggctgctcccttggtcgcactagctgactacattgcccataggcaaaactaa

SEQ ID NO: 71 - DNA, 1191 bp, Arabidopsis thaliana
  source 1..1191: mol_type = genomic DNA
atggcaagtagtatgcttagttccgcaacaatggttgcaagtccagctcaagccactatggttgcaccattcaacgggctgaagtcatcagcagcattccctgccacccgtaaggctaataacgacattacaagtattacttcaaatggtggaagggtcaatatggccgacctcaagagtacttttttagatgtgtatagtgttctcaagagtgatctcctccaggatcctagctttgagtttacccatgagtctcgacaatggctcgaacgaatgttggactataacgtgcgaggtggaaagctgaaccgtggcctaagcgtggtggatagttataaactgctgaaacaggggcaagatctaactgaaaaggagacatttctatcatgtgccctcggttggtgtatcgaatggctacaagcttatttcttagtactcgatgatataatggacaacagtgtcacaaggcgtggacagccatgttggtttaggaaacccaaggtcggcatgattgctataaacgatgggatccttctgagaaatcacatacataggattcttaaaaagcactttcgagagatgccctattacgtagatctcgtcgatctctttaacgaggtcgaattccagactgcatgtggccaaatgattgaccttattaccacttttgatggtgaaaaggacctttctaagtactctttgcaaattcatcgtcgtatcgtggagtacaaaaccgcatactacagcttttaccttccagtcgcctgcgctctcctgatggctggagaaaacctggaaaatcacacagatgtgaaaacagtgctcgtggatatgggtatatacttccaggtccaagacgattatctcgactgtttcgcagatccagaaactcttggaaagattggcactgacatagaagattttaaatgctcatggttggttgtcaaagcattagaaagatgctcagaagaacagaccaaaattctgtatgagaattacggcaaggccgagccctctaacgttgctaaggtcaaagctttgtacaaagaactagacctcgagggggcttttatggagtacgaaaaagaaagctacgagaaactcacaaagctcatagaagcacatcaaagtaaagccatacaggcagtcctcaagtctttccttgctaagatctacaagaggcagaagtaa

SEQ ID NO: 72 - DNA, 1905 bp, Sorghum bicolor
  source 1..1905: mol_type = genomic DNA
atggcttcttcaatgctctcaagtgcaaccatggttgcatctcctgcacaagcaaccatggtcgccccattcaatggattaaaaagcagcgcagccttccctgcaactagaaaagcaaataacgatataacttcaataacctctaatggtggaagagtaaacatggcccccatcaccgttgctgctgcaccacctcccgctagtctagaaactggtggcggcgctggcctaacagttgcaactgtgggggcattcgagccttgtgcctggggtgacttttttgttacatacgtgcctccagtctcacaagaatcagaggaacgaatgagggaaagggcacatcagctaaaagctgagttacgaaggcgtatgagtttcgaagcaacccgttcagtagctgacatggtcacccttgttgatacattgcaaaggctcggaattgacaaccattttaggcacgaggtggacgccgctctgaagcgtatcaataacaataactcttgtgagtccctgggtgacagttctgcaggctctgggggtggaggtgccgcagacgacggcctccataccgttgcattgaggttccgattgttgaggcagcatggcgtttgggtgcccgcagacgtattcgaccgtttcaaagacgataccacaggaggattctcagaatccctctcatctgatcctagaggactcctatccttatataatgctgctcatatggccactccaggagagcagggcctggatgaagccatttcttttgcccgtaggcatcttgagtcactgaaattaaaaggcactctttctagcccattggctgaacaggtgtgccgtgccttggatatcccccttgcaaggttacctaagcgtctggagactatgcactatgtcgttgagtacgagaaggaggaaggtcacgacgctgtattacttgagttggccaggctagattttgacctagtgcgatttttacacctaagagagcttaaggacttatccttatggtggaaggatctctacgggaatgtaaaactcaattacgctagagatcgactcgttgaaaattatttttggacttgcggtgtttttcatgaggaggaatacagtcgagctcgaatgctgtttgctaaaactttcgggttgctgtccctgatggacgatacatatgatgtctatgctactttagaagaatgtcatatattgaacgacgctatacagcgatgggacgaaaactcagcatccatcctcccagaatatatgaggatgttctatattaatctagtaaggaacttccagggctttgaggactctctccaacccaatgagaagtatcgtgtatcctatgccaagcaagcttttaaattatccagcaagtattacttggatgaggctaaatggtgtagtgaaaagtacgcccctagtttcaaagagcacatggaagtgtcagtcatgtccagtgggttccccaccctagctgtagttctcctaatgggggccggggatatggcaaccagggaagctttcaggtgggctattggtgtaccagctgtggtgtcagccagtggcgaggtggccaggtttcgaaacgatattgcttcctacaaaaaaggcaagaataaaaaagacgttgcctccagtgtcgagtgttacgctaaggaacatggaacaagtggagaagaggctgctgtcgctatcgccgggatggcagaacatgcatggcgaacaatcaacagaagttgtatggaaatggatagcgcactactaccagccgcccagctagtggtgaatctaaccaaaaccctggaagtaatttatctaggcggtcgtgacgcatatacattcgcagccgacctaaaggatttggtcgtttccctatttctaaacgggccaaccaccgtctag

SEQ ID NO: 73 - DNA, 1800 bp, Prunella vulgaris
  source 1..1800: mol_type = genomic DNA
atgtcctccctgagcatacccttctcatcagccatctgcacatcttccatccccaagatatcaactggccaccacaggcgaacagctaggatgcctgcccacgacacatcaaggctcgtgtttcgtcccagcgctgtgatggtcgagggtagtcctatgactactagctccaacggcaaagaagtgcagcgattaataaccaccttcaagccatcaatgtggaaagacattttcagtacattcagcttcgacaaccaggttcaggagaagtacttgaaagaaattgaggaactaaaaaaagaggttaggtctactttgatgagcgccacacaccgaaaattattcgatctaatagataacctagaaagaatgggcattgcctatcactttgaaacagaaattgaggacaagctaaagcaggcccatgcctctttggaggaggaggatgattatgatctcttcacaacagccctaagattcaggttactgcgtcaacataggtatcacgtttcctgtgacccattcgcaaaatttgtggaccaagacaacaagttgaaggagagcctctcttccgacgtggagggattattatcactattcgaggcaagtcatttaagaatccataacgaagatgtcttagatgaggcaatcgtttttacaacacatcacttaaatcgaatgatgccacaacttgaatcaccattgaaggaagaggtgaaacatgccttgaggtaccccttgcataaatgcctgggaatattaagtttgcgttttcacatcgataggtacgaaaatgataagtctagagatgaagtagtccttcgactaggtcaagtaaacttcaactacatgcagaacatttatatgaacgaattatacgaaataaccacttggtggaacaaattgcagatgacctcaaaagtcccatacttcagagacagactagtcgagtgttacatgtgggggcttgcttaccacttcgagccagagtacgctccagtcagggtgctcattaccaaatactatatgactgctaccaccgtagatgacacttacgataactatgccactctagaggaaatcgagttgtttacccaggccatcgacaggtggtcagaagatgagatagaccagttgcccgacgaatacctaaagatagtctataaaggtcttatgaatttcaccgaagaatttagaagggatgctgaggagaggggcaagggatatgtcataccttattttattgaagagacaaagagagccacacaagggtatgcaaacgaacagcgttggataatgaagcgtgagatgccctcattcgaggagtatatggtaaacagtagggtaacatctctcatgtatgtgacctacgttgcagtcgttgcagtaatcgaatctgcaacaaaggaaaccgttgattgggctctttctgacagcgacatattcgtctatacaaacgatatcgggcgtctaattgatgatctagcaactcacaggagggaacgaaaggatggtaccatgctcacctcaatggattattacatgaaggagtatggcggtaccatggaggagggggaggccgcctttcgtaagttaatggaagaaaaatggaagctcctgaacgcagcctgggttgataccatcaacggtaaggagtccaaagagattgttgtccaagtactggacctcgctaggatttgtggcacactgtacggcgacgaagaggatggttttacctatcccgagaaaaacttcgctcccctggtcgctgccctattaatgaaccccattcatatttag

SEQ ID NO: 74 - DNA, 1830 bp, Daphne genkwa
  source 1..1830: mol_type = genomic DNA
atgagctgttttcaggccctgcatagctctcctaccacaacaacttccagtagtgcagcccttctgttccctgcagcagctgttagtgaacttcagaactccaaatgctgtttctcacagtactctctcgtcctgggaaccagatcagtccccggacccgccccactgcacgccatcgctgtcgacactcaacccagacgatccgctgattatacccctaacatctggggtaaccattttatagccaccagtccaactgctcttggaattgagtccgaagtggaggagcatgaaaagctaaaacaggaggttaggggagtcatagcagatgcaatggaagatcctaacaagaatctggacttgatagacgccattcagaggctgggcgtgagctatcacttcgaagaagaggtagaagagtttcttcaaaagacacacgctaatcttcatggattcattaaagataatgcagacaacctacagcagatatctttatgttttcgactgcttcgacaacatggttatcaagtgtcctgtaatatgtttaccaactttatcgaggacgatgggagtttccgtggttccttaaaacaagactttgagggtttgatgtctctttatgaggccgcacacctacgtgtccatggcgaagaaatactggaaaatgccttggcattttcaactacacaactgaaacttattgctaaagccggctccaaattttctgctataataaaccatagcttgcgaaatcctatccgtaagaacttccccaggcttgacactaggttctacatatccgtgtaccaggaaatcaaacccaactcacataatccatacctattgcagttcagcaagctggactttaacctagtacaaaagcaatatcgagaggaacttaatcagatcagtcgatggtggaacgagttagacgcagaaaccaaattcccattcgcccgtaacagaattgttgaggcattcttcttcatcatggggacctatttcgagcccagatacaagttgggtcgaaaaatagctactaaggctctagccatgctaacaattcttgacgatatctacgacgtacatggaaccccccaggagcttgacctcttgacccaggcattagaaagatgggaccctaatctagccagcgaaatgccagaatacattaaaaacttttatgtagctctccttgacacatttaaggaccttgaggaagatatgggcaccgcccaaggggcagacgaccatgtggccgataaaataaagtattgtaaggaggggctaattaaggtcgctaaagcctaccataccgaagccaagtggtatgagcaaaattacaccccaaccatggaagaatacatggaggttgctctggtcagcgtggctacccctttcattggtgccatggtattactgggcatgggtcatctagctaataaggacgctttcgaatggttcaggactcatcccaaaatagtggcctcagcatgcctaataccccgacttcaggatgacatcgtgggtcacgaatttgagcagaaaagaggtcattccgcatcttgcgttgactgttatgtaaaagaacatggaaagggaaaagaagaagccgtagaggcattttataaacaagccagtgatgcctggaaggacgctaacgctgactttatcaggccaaccgctgtccctatgcaagtgcttaccacccttttgaacatctcaaggatagcccatgtagtatataacgattgtgatggctacgccgacccccacgcaattaaggctttggttgcctcagtatttgtacatcctatcccttataactga

SEQ ID NO: 75 - DNA, 1503 bp, Euphorbia lathyris
  source 1..1503: mol_type = genomic DNA
atggatcttcagctacagatcccatcttaccccatcattttcagcttctttatatttatctttatgctgattaagatatggaagaagcaaacccagaccagcatcttccctcctggccccttcaaattcccaatagttggcaacataccacaactcgctactggcggtacacttcctcaccatcgactccgtgaccttgcaaaaatttatggtcccataatgacaattcaattaggtcaggtcaaaagcgtagttattagtagccctgaaactgctaaggaggtacttaagactcaggacatccagtttgccgacaggccactactcctagctggagagatggttctttacaatcgtaaggacatcttgtacggcacatacggagatcaatggcgacagatgcgaaaaatctgtacactcgagctgttatctgctaaaaggattcagtcctttaagtccgtaagagaaaaggaagtagaaagtttcattaaaacattgagatctaagtccggtatcccagttaatctcactaacgccgtctttgagttgaccaataccattatgatgataaccactatcggacaaaagtgcaaaaaccaggaggctgtaatgtcagtaattgacagggtgtctgaggcagccgctggcttttctgtcgcagatgtttttccctctttgaagtttctgcattacctgtcaggagaaaagaccaaactacaaaagctccacaaagagactgaccagatacttgaggagatcatttccgagcacaaggctaatgccaaggtaggagcccaagctgataacttactggatgtgttactggacttgcaaaaaaacggtaatctccaggtacctctcactaacgataatattaaagcagcaactctggagatgttcggcgcaggatcagatacatctagcaaaactacagactgggctatggcccaaatgatgaggaagcctaccacaatgaagaaggcccaggaggaggtacgaagggttttcggagaaaacggcaaagtagaggaatccaggatccaggagctgaaatatcttaagttggtagtaaaagagacactcaggcttcaccctgccgtcgcattgataccccgagaatgcagggagaaaactaagattgatggattcgacatctacccaaagactaaaattctcgttaatccatgggcaattggacgtgatcctaaggtgtggaatgagccagagtcatttaatccagagaggttccaggatagcccaatcgattataagggtactaacttcgaactaattccttttggagcaggaaaacgtatatgtcccggcatgaccctgggcataactaacctggaattatttctcgctaatctgctataccatttcgattggaagtttcctgatggtattacttcagagaatctcgacatgaccgaagccatcggaggtgccattaagcgaaagctcgacttggaactcatatccataccatatacatccagctag

SEQ ID NO: 76 - protein, 773 aa, Plectranthus barbatus
MKMLMIKSQFRVHSIVSAWANNSNKRQSLGHQIRRKQRSQVTECRVASLDALNGIQKVGPATIGTPEEENKKIEDSIEYVKELLKTMGDGRISVSPYDTAIVALIKDLEGGDGPEFPSCLEWIAQNQLADGSWGDHFFCIYDRVVNTAACVVALKSWNVHADKIEKGAVYLKENVHKLKDGKIEHMPAGFEFVVPATLERAKALGIKGLPYDDPFIREIYSAKQTRLTKIPKGMIYESPTSLLYSLDGLEGLEWDKILKLQSADGSFITSVSSTAFVFMHTNDLKCHAFIKNALTNCNGGVPHTYPVDIFARLWAVDRLQRLGISRFFEPEIKYLMDHINNVWREKGVFSSRHSQFADIDDTSMGIRLLKMHGYNVNPNALEHFKQKDGKFTCYADQHIESPSPMYNLYRAAQLRFPGEEILQQALQFAYNFLHENLASNHFQEKWVISDHLIDEVRIGLKMPWYATLPRVEASYYLQHYGGSSDVWIGKTLYRMPEISNDTYKILAQLDFNKCQAQHQLEWMSMKEWYQSNNVKEFGISKKELLLAYFLAAATMFEPERTQERIMWAKTQVVSRMITSFLNKENTMSFDLKIALLTQPQHQINGSEMKNGLAQTLPAAFRQLLKEFDKYTRHQLRNTWNKWLMKLKQGDDNGGADAELLANTLNICAGHNEDILSHYEYTALSSLTNKICQRLSQIQDKKMLEIEEGSIKDKEMELEIQTLVKLVLQETSGGIDRNIKQTFLSVFKTFYYRAYHDAKTIDAHIFQVLFEPVV

SEQ ID NO: 77 - protein, 579 aa, Origanum majorana
MASLAFTPGAATFSGNVVRRRKDNFPVHGFPTTIRSSVSVTVKCYVSTTNLMVKIKEKFKGKNVNSLTVEAADDDMPSNLCIIDTLQRLGIDRYFQPQVDSVLDHAYKLWQGKEKDTVYSDISIHAMAFRLLRVKGYQVSSEELDPYIDVERMKKLKTVDVPTVIELYRAAQERMYEEEGSLERLHVWSTNFLMHQLQANSIPDEKLHKLVEYYLKNYHGILDRVGVRRNLDLFDISHYPTLRARVPNLCTEDFLSFAKEDFNTCQAQHQKEHEQLQRWFEDCRFDTLKFGRETAVGAAHFLSSAILGESELCNVRLALAKHMVLVVFIDDFFDHYGSREDSFKILHLLKEWKEKPAGEYGSEEVEILFTAVYNTVNELAEMAHVEQGRNIKGFLIELWVEIVSIFKIELDTWSNDTTLTLDEYLSSSWVSVGCRICILVSMQLLGVQLTDEMLLSDECINLCKHVSMVDRLLNDVGTFEKERKENTGNSVSLLLAAAVKEGRPITEEEAIIKIKKMAENERRKLMQIVYKRESVFPRKCKDMFLKVCRIGCYLYASGDEFTSPQKMKEDVKSLIYESL

SEQ ID NO: 78 - protein, 722 aa, Plectranthus barbatus
MASCGAIGSSFLPLLHSDESSLLSRPTAALHIKKQKFSVGAALYQDNTNDVVPSGEGLTRQKPRTLSFTGEKPSTPILDTINYPIHMKNLSVEELEILADELREEIVYTVSKTGGHLSSSLGVSELTVALHHVFNTPDDKIIWDVGHQAYPHKILTGRRSRMHTIRQTFGLAGFPKRDESPHDAFGAGHSSTSISAGLGMAVGRDLLQKNNHVISVIGDGAMTAGQAYEAMNNAGFLDSNLIIVLNDNKQVSLPTATVDGPAPPVGALSKALTKLQASRKFRQLREAAKGMTKQMGNQAHEIASKVDTYVKGMMGKPGASLFEELGIYYIGPVDGHNIEDLVYIFKKVKEMPAPGPVLIHIITEKGKGYPPAEVAADKMHGVVKFDPTTGKQMKVKTKTQSYTQYFAESLVAEAEQDEKVVAIHAAMGGGTGLNIFQKRFPDRCFDVGIAEQHAVTFAAGLATEGLKPFCTIYSSFLQRGYDQVVHDVDLQKLPVRFMMDRAGLVGADGPTHCGAFDTTYMACLPNMVVMAPSDEAELMHMVATAAVIDDRPSCVRYPRGNGIGVPLPPNNKGIPLEVGKGRILKEGNRVAILGFGTIVQNCLAAAQLLQEHGISVSVADARFCKPLDGDLIKNLVKEHEVLITVEEGSIGGFSAHVSHFLSLNGLLDGNLKWRPMVLPDRYIDHGAYPDQIEEAGLSSKHIAGTVLSLIGGGKDSLHLINM

SEQ ID NO: 79 - protein, 360 aa, Plectranthus barbatus
MRSMNLVDAWVQNLPIFKQPHPSKFIHHPRFEPAFLKSRRPISSFAVSAVLTGEEARIFTRGDEAPFNFNAYVVEKATHVNKALDDAVAVKNPPMIHEAMRYSLLAGGKRVRPMLCIAACEVVGGPQAAAIPAACAVEMIHTMSLIHDDLPCMDNDDLRRGKPTNHKVFGENVAVLAGDALLAFAFEFIATATTGVAPERILAAVAELAKAIGTEGLVAGQVVDLHCTGNPNVGLDTLEFIHIHKTAALLEASVVLGAILGGGSSDQVEKLRTFARKIGLLFQVVDDILDVTKSSEELGKTAGKDLAVDKTTYPKLLGLEKAMEFAERLNEEAKQQLLDFDPRKAAPLVALADYIAHRQN

SEQ ID NO: 80 - protein, 396 aa, Arabidopsis thaliana
MASSMLSSATMVASPAQATMVAPFNGLKSSAAFPATRKANNDITSITSNGGRVNMADLKSTFLDVYSVLKSDLLQDPSFEFTHESRQWLERMLDYNVRGGKLNRGLSVVDSYKLLKQGQDLTEKETFLSCALGWCIEWLQAYFLVLDDIMDNSVTRRGQPCWFRKPKVGMIAINDGILLRNHIHRILKKHFREMPYYVDLVDLFNEVEFQTACGQMIDLITTFDGEKDLSKYSLQIHRRIVEYKTAYYSFYLPVACALLMAGENLENHTDVKTVLVDMGIYFQVQDDYLDCFADPETLGKIGTDIEDFKCSWLVVKALERCSEEQTKILYENYGKAEPSNVAKVKALYKELDLEGAFMEYEKESYEKLTKLIEAHQSKAIQAVLKSFLAKIYKRQK

SEQ ID NO: 81 - protein, 634 aa, Sorghum bicolor
MASSMLSSATMVASPAQATMVAPFNGLKSSAAFPATRKANNDITSITSNGGRVNMAPITVAAAPPPASLETGGGAGLTVATVGAFEPCAWGDFFVTYVPPVSQESEERMRERAHQLKAELRRRMSFEATRSVADMVTLVDTLQRLGIDNHFRHEVDAALKRINNNNSCESLGDSSAGSGGGGAADDGLHTVALRFRLLRQHGVWVPADVFDRFKDDTTGGFSESLSSDPRGLLSLYNAAHMATPGEQGLDEAISFARRHLESLKLKGTLSSPLAEQVCRALDIPLARLPKRLETMHYVVEYEKEEGHDAVLLELARLDFDLVRFLHLRELKDLSLWWKDLYGNVKLNYARDRLVENYFWTCGVFHEEEYSRARMLFAKTFGLLSLMDDTYDVYATLEECHILNDAIQRWDENSASILPEYMRMFYINLVRNFQGFEDSLQPNEKYRVSYAKQAFKLSSKYYLDEAKWCSEKYAPSFKEHMEVSVMSSGFPTLAVVLLMGAGDMATREAFRWAIGVPAVVSASGEVARFRNDIASYKKGKNKKDVASSVECYAKEHGTSGEEAAVAIAGMAEHAWRTINRSCMEMDSALLPAAQLVVNLTKTLEVIYLGGRDAYTFAADLKDLVVSLFLNGPTTV

SEQ ID NO: 82 - protein, 599 aa, Prunella vulgaris
MSSLSIPFSSAICTSSIPKISTGHHRRTARMPAHDTSRLVFRPSAVMVEGSPMTTSSNGKEVQRLITTFKPSMWKDIFSTFSFDNQVQEKYLKEIEELKKEVRSTLMSATHRKLFDLIDNLERMGIAYHFETEIEDKLKQAHASLEEEDDYDLFTTALRFRLLRQHRYHVSCDPFAKFVDQDNKLKESLSSDVEGLLSLFEASHLRIHNEDVLDEAIVFTTHHLNRMMPQLESPLKEEVKHALRYPLHKCLGILSLRFHIDRYENDKSRDEVVLRLGQVNFNYMQNIYMNELYEITTWWNKLQMTSKVPYFRDRLVECYMWGLAYHFEPEYAPVRVLITKYYMTATTVDDTYDNYATLEEIELFTQAIDRWSEDEIDQLPDEYLKIVYKGLMNFTEEFRRDAEERGKGYVIPYFIEETKRATQGYANEQRWIMKREMPSFEEYMVNSRVTSLMYVTYVAVVAVIESATKETVDWALSDSDIFVYTNDIGRLIDDLATHRRERKDGTMLTSMDYYMKEYGGTMEEGEAAFRKLMEEKWKLLNAAWVDTINGKESKEIVVQVLDLARICGTLYGDEEDGFTYPEKNFAPLVAALLMNPIHI

SEQ ID NO: 83 - protein, 609 aa, Daphne genkwa
MSCFQALHSSPTTTTSSSAALLFPAAAVSELQNSKCCFSQYSLVLGTRSVPGPAPLHAIAVDTQPRRSADYTPNIWGNHFIATSPTALGIESEVEEHEKLKQEVRGVIADAMEDPNKNLDLIDAIQRLGVSYHFEEEVEEFLQKTHANLHGFIKDNADNLQQISLCFRLLRQHGYQVSCNMFTNFIEDDGSFRGSLKQDFEGLMSLYEAAHLRVHGEEILENALAFSTTQLKLIAKAGSKFSAIINHSLRNPIRKNFPRLDTRFYISVYQEIKPNSHNPYLLQFSKLDFNLVQKQYREELNQISRWWNELDAETKFPFARNRIVEAFFFIMGTYFEPRYKLGRKIATKALAMLTILDDIYDVHGTPQELDLLTQALERWDPNLASEMPEYIKNFYVALLDTFKDLEEDMGTAQGADDHVADKIKYCKEGLIKVAKAYHTEAKWYEQNYTPTMEEYMEVALVSVATPFIGAMVLLGMGHLANKDAFEWFRTHPKIVASACLIPRLQDDIVGHEFEQKRGHSASCVDCYVKEHGKGKEEAVEAFYKQASDAWKDANADFIRPTAVPMQVLTTLLNISRIAHVVYNDCDGYADPHAIKALVASVFVHPIPYN

SEQ ID NO: 84 - protein, 500 aa, Euphorbia lathyris
MDLQLQIPSYPIIFSFFIFIFMLIKIWKKQTQTSIFPPGPFKFPIVGNIPQLATGGTLPHHRLRDLAKIYGPIMTIQLGQVKSVVISSPETAKEVLKTQDIQFADRPLLLAGEMVLYNRKDILYGTYGDQWRQMRKICTLELLSAKRIQSFKSVREKEVESFIKTLRSKSGIPVNLTNAVFELTNTIMMITTIGQKCKNQEAVMSVIDRVSEAAAGFSVADVFPSLKFLHYLSGEKTKLQKLHKETDQILEEIISEHKANAKVGAQADNLLDVLLDLQKNGNLQVPLTNDNIKAATLEMFGAGSDTSSKTTDWAMAQMMRKPTTMKKAQEEVRRVFGENGKVEESRIQELKYLKLVVKETLRLHPAVALIPRECREKTKIDGFDIYPKTKILVNPWAIGRDPKVWNEPESFNPERFQDSPIDYKGTNFELIPFGAGKRICPGMTLGITNLELFLANLLYHFDWKFPDGITSENLDMTEAIGGAIKRKLDLELISIPYTSS

(19) United States
(12) Patent Application Publication
(10) Pub. No.: US 2025/0333756 A1
(43) Pub. Date: Oct. 30, 2025

(54) ENGINEERING TOMATO FRUITS AS A PRODUCTION PLATFORM FOR TERPENOID PRODUCTS

(71) Applicants: BOARD OF TRUSTEES OF MICHIGAN STATE UNIVERSITY, East Lansing, MI (US); REGENTS OF THE UNIVERSITY OF MINNESOTA, Minneapolis, MN (US); UNIVERSITY OF GEORGIA RESEARCH FOUNDATION, INC., Athens, GA (US)

(72) Inventors: Björn Hamberger, Okemos, MI (US); Lucas Reist, Lansing, MI (US); Robin Buell, Athens, GA (US); Daniel Voytas, St. Paul, MN (US); Natalie Christine Deans, Athens, GA (US); Jon Cody, St. Paul, MN (US)

(21) Appl. No.: 19/192,111

(22) Filed: Apr. 28, 2025

Related U.S. Application Data
(60) Provisional application No. 63/639,464, filed on Apr. 26, 2024.

(51) Int. Cl. C12N 15/82; C12N 9/02; C12N 9/10; C12N 9/88; C12N 15/52

(52) U.S. Cl. CPC C12N 15/825; C12N 9/0071; C12N 9/1051; C12N 9/88; C12N 15/52; C12Y 204/0117

(57) ABSTRACT

Disclosed herein modified tomato plants, plant cells, plant parts, plant seeds, and fruit comprising targeted modifications to endogenous terpene or terpenoid biosynthetic genes resulting in decreased expression of the endogenous terpene or terpenoid biosynthetic gene relative to a reference plant lacking the modification. The decrease of expression in the endogenous terpene or terpenoid biosynthetic gene results in a depletion of carotene in the fruit of the tomato relative to the reference tomato plant lacking the modification. The disclosure further relates to expression of heterologous terpene or terpenoid biosynthetic genes in the modified tomato plants, and related methods and uses.

### CROSS-REFERENCE TO RELATED APPLICATIONS

This patent application claims the benefit of U.S. Provisional Patent Application Ser. No. 63/639,464, filed on Apr. 26, 2024, the disclosure of which is incorporated herein by reference in its entirety.

### FEDERAL FUNDING

This invention was made with government support under grant number 2126592 awarded by the National Science Foundation. The government has certain rights in the invention.

### INCORPORATION OF SEQUENCE LISTING

The instant application contains a Sequence Listing named “P14775US01.xml” which has been submitted electronically in XML format, is 215,562 bytes in size, was created on Apr. 23, 2025, and is herein incorporated by reference in its entirety.

### BACKGROUND

Plants produce a broad spectrum of specialized metabolites that function in adaptation and interaction with their environment. One of these classes of metabolites is the terpenoids for which over 50,000 are known and are used by humans as pharmaceuticals, fragrance, food additives, agrichemicals, and chemical feedstocks. However, currently available methods for petrochemical synthesis, extraction, and purification of terpenoids from the native plant sources have limited economic sustainability.

Terpenoids, represent the largest and most complex class of specialized metabolites with over 86,000 reported natural compounds found in plants and many living organisms (Dictionary of Natural Products 31.1. Terpenoids are useful in applications spanning medicine, nutrition, agriculture, flavors, fragrances, colorants and cosmetics. All terpenoids are biosynthesized from the two C5 isoprenoid building blocks isopentenyl diphosphate (IPP) and its isomer dimethylallyl diphosphate (DMAPP). The condensation of these precursors generates larger isoprenoid molecules including geranyl diphosphate (GPP, C10), farnesyl diphosphate (FPP, C15), and geranylgeranyl diphosphate (GGPP, C20) which are first cyclized by terpene synthases to form the diverse terpene scaffolds and subsequently functionalized by cytochromes P450 into terpenoids (Chatzivasileiou et al. 2019). Achieving sustainable bioproduction requires the iterative design, construction and testing biological systems capable of converting renewable feedstock into bioproducts at maximum yield and productivity.

### SUMMARY

Described herein are modified tomato plants (Solanum lycopersicum), plant tissues or parts, fruits of the tomato, and tomato seeds having one or more modified endogenous terpene or terpenoid biosynthetic genes. Methods and compositions described herein provide modified tomato plants that biosynthesize minimal terpenoids to serve as a chassis for expression of high value terpenoids. The modified tomato plants comprise at least one targeted modification in at least one endogenous terpene or terpenoid biosynthetic gene resulting in decreased expression of the endogenous terpene or terpenoid biosynthetic gene relative to a reference plant lacking the modification. The targeted modification comprises an insertion, replacement, and/or deletion of one or more nucleotides in the endogenous terpene or terpenoid biosynthetic genes. Decrease expression results in depletion of carotene in fruit of the tomato relative to the reference tomato plant lacking the modification.

In certain embodiments, the present disclosure provides modified tomato plants, wherein the endogenous terpene or terpenoid biosynthetic genes with decreased expression comprise: (a) a beta carotene hydroxylase, a phytoene synthase, a cytochrome P450, a uracil dependent glucosyl transferase, or a combination thereof, (b) a DNA molecule having at least 90%, at least 95%, at least 96%, at least 97%, at least 98%, at least 99%, or 100% sequence identity to the nucleic acid sequence set forth in any one of SEQ ID NOs: 1-44 or an allelic variant thereof, (c) a beta carotene hydroxylase (CrtR-B2; SEQ ID NO: 2 or 24), phytoene synthase (PSY1; SEQ ID NO: 1 or 23), cytochrome P450_1 (P450_1; SEQ ID NO: 5 or 27), cytochrome P450_2 (P450_2; SEQ ID NO: 6 or 28), cytochrome P450_3 (P450_3; SEQ ID NO: 7 or 29), cytochrome P450_4 (P450_4; SEQ ID NO: 8 or 30), and uracil dependent glucosyl transferase 4 (UGT_4; SEQ ID NO: 17 or 39), or (d) uracil dependent glucosyl transferase-6 (UGT_6; SEQ ID NO: 19 or 41), uracil dependent glucosyl transferase-7 (UGT_7; SEQ ID NO: 20 or 42), uracil dependent glucosyl transferase-8 (UGT_8; SEQ ID NO: 21 or 43), uracil dependent glucosyl transferase-9 (UGT_9; SEQ ID NO: 22 or 44), cytochrome P450_8 (P450_8; SEQ ID NO: 12 or 34), and cytochrome P450_9 (P450_9; SEQ ID NO: 13 or 35).

In certain embodiments, the present disclosure provides modified tomato plants with at least one targeted modification in at least one endogenous terpene or terpenoid biosynthetic gene resulting in decreased expression of the endogenous terpene or terpenoid biosynthetic gene, wherein the modified tomato plant comprises a heterologous terpene or terpenoid biosynthetic gene. In various embodiments, the heterologous terpene or terpenoid biosynthetic gene comprises: (a) a heterologous terpene synthase, cytochrome P450, uracil dependent glucosyl transferase, or a combination thereof, (b) a nucleotide sequence having at least 90%, at least 95%, at least 96%, at least 97%, at least 98%, at least 99%, or 100% sequence identity to one or more of SEQ ID NOs: 67-75, or (c) CfTPS2 (SEQ ID NO: 67), OmTPS3 (SEQ ID NO: 68), C/DXS (SEQ ID NO: 69), C/GPPS (SEQ ID NO: 70), TP-AtFPPS (SEQ ID NO: 71), TP-SbTPS10 (SEQ ID NO: 72), PvHVS (SEQ ID NO: 73), DgTPS1 (SEQ ID NO: 74), ElCYP726A27 (SEQ ID NO: 75), and combinations thereof.

Within other related embodiments, the present disclosure provides methods of producing a modified tomato plant material, comprising introducing a targeted modification into at least one endogenous terpene or terpenoid biosynthetic gene into a tomato plant to produce a modified tomato plant with decreased expression of the endogenous terpenoid biosynthetic gene relative to a reference tomato plant lacking the modification, wherein the targeted modification in the endogenous terpenoid biosynthetic gene comprises an insertion, replacement, and/or deletion of one or more nucleotides in the endogenous terpenoid biosynthetic gene, and wherein the decrease of expression in the endogenous terpenoid biosynthetic gene results in a depletion of carotene in fruit of the tomato plant relative to the reference tomato plant lacking the modification. In some embodiments, the targeted modification can be at a genomic locus comprising the at least one endogenous terpene or terpenoid biosynthetic gene, within the coding region, non-coding region, regulatory sequence, or untranslated region of the endogenous terpene or terpenoid biosynthetic gene. Methods include targeted DNA modification through use of an RNA-guided endonuclease (e.g. Cas9) and a guide RNA.

In various embodiments, methods include modifying tomato endogenous terpene or terpenoid biosynthetic genes comprising: (a) a beta carotene hydroxylase, a phytoene synthase, a cytochrome P450, a uracil dependent glucosyl transferase, or a combination thereof, (b) a DNA molecule having at least 90%, at least 95%, at least 96%, at least 97%, at least 98%, at least 99%, or 100% sequence identity to the nucleic acid sequence set forth in any one of SEQ ID NOs: 1-44 or an allelic variant thereof, (c) a beta carotene hydroxylase (CrtR-B2; SEQ ID NO: 2 or 24), phytoene synthase (PSY1; SEQ ID NO: 1 or 23), cytochrome P450_1 (P450_1; SEQ ID NO: 5 or 27), cytochrome P450_2 (P450_2; SEQ ID NO: 6 or 28), cytochrome P450_3 (P450_3; SEQ ID NO: 7 or 29), cytochrome P450_4 (P450_4; SEQ ID NO: 8 or 30), and uracil dependent glucosyl transferase-4 (UGT_4; SEQ ID NO: 17 or 39), or (d) uracil dependent glucosyl transferase-6 (UGT_6; SEQ ID NO: 19 or 41), uracil dependent glucosyl transferase-7 (UGT_7; SEQ ID NO: 20 or 42), uracil dependent glucosyl transferase-8 (UGT_8; SEQ ID NO: 21 or 43), uracil dependent glucosyl transferase-9 (UGT_9; SEQ ID NO: 22 or 44), cytochrome P450_8 (P450_8; SEQ ID NO: 12 or 34), and cytochrome P450_9 (P450_9; SEQ ID NO: 13 or 35). Methods are provided for modifying the tomato plant to further comprise a heterologous terpene or terpenoid biosynthetic gene comprising: (a) a heterologous terpene synthase, cytochrome P450, uracil dependent glucosyl transferase, or a combination thereof, (b) a nucleotide sequence having at least 90%, at least 95%, at least 96%, at least 97%, at least 98%, at least 99%, or 100% sequence identity to one or more of SEQ ID NOs: 67-75, or (c) CfTPS2 (SEQ ID NO: 67), OmTPS3 (SEQ ID NO: 68), CfDXS (SEQ ID NO: 69), CfGPPS (SEQ ID NO: 70), TP-AtFPPS (SEQ ID NO: 71), TP-SbTPS10 (SEQ ID NO: 72), PvHVS (SEQ ID NO: 73), DgTPS1 (SEQ ID NO: 74), ElCYP726A27 (SEQ ID NO: 75), and combinations thereof. Methods for isolating heterologous terpenes or terpenoids from the fruit of the modified tomato plant are provided.

These and other related aspects of the present disclosure will be better understood in view of the following detailed description, which exemplify certain aspects of the various embodiments disclosed herein.

### BRIEF DESCRIPTION OF THE DRAWINGS

FIG. 1 is a graph showing reduced gene expression of modified endogenous phytoene synthase (Psy1; SEQ ID NO: 1 or 23), endogenous cytochrome P450_8 (P450_8; SEQ ID NO: 12 or 34), or endogenous cytochrome P450_9 (P450_9; SEQ ID NO: 12 or 35) in tomato fruit without modification (−) or modified using a Virus Induced Gene Silencing (VIGS) method for gene transcript suppression.

FIG. 2 is a graph showing reduced gene expression of candidate endogenous terpene or terpenoid biosynthetic genes in different assays.

FIG. 3 is a graph showing knockdown of gene expression of candidate endogenous terpene or terpenoid biosynthetic genes Tobacco rattle virus (TRV)-derived siRNAs.

FIG. 4 is a heatmap showing loss of candidate endogenous cytochrome P450s results in changes in metabolite profiles.

FIG. 5 is a table showing candidate combinations of endogenous terpene or terpenoid biosynthetic genes knockouts.

FIG. 6 is a heatmap showing Sanger genotyping data of candidate combinations of endogenous terpene or terpenoid biosynthetic genes knockouts in tomato To generation.

FIG. 7 shows images of tomato flowers with the yellow phenotype of wild type tomato flowers and the white phenotype of tomato flowers with psyl mutations.

FIGS. 8A and 8B show genotypes of modified tomato plants with mutations of the endogenous terpene or terpenoid biosynthetic gene alleles shown and crossed to produce tomato plants with combined mutations.

FIG. 9 is a gas chromatogram showing production of heterologous casbene in tomato fruit expressing (1) heterologous 1-deoxy-D-xylulose-5-phosphate synthase (CfDXS; SEQ ID NO: 69) and geranylgeranyl pyrophosphate synthase ((AGGPPS; SEQ ID NO: 70), or (2) heterologous 1-deoxy-D-xylulose-5-phosphate synthase (CfDXS; SEQ ID NO: 69), geranylgeranyl pyrophosphate synthase (CfGGPPS; SEQ ID NO: 70), and terpene synthase 1 (DgTPS1; SEQ ID NO: 74).

FIG. 10 is a gas chromatogram showing production of heterologous 11-hydroxy vulgarisane in tomato fruit expressing (1) heterologous 1-deoxy-D-xylulose-5-phosphate synthase (CfDXS; SEQ ID NO: 69) and geranylgeranyl pyrophosphate synthase (CfGGPPS; SEQ ID NO: 70), or (2) heterologous 1-deoxy-D-xylulose-5-phosphate synthase (CfDXS; SEQ ID NO: 69), geranylgeranyl pyrophosphate synthase (CfGGPPS; SEQ ID NO: 70), and 11-hydroxyvulgarisane synthase (PvHVS; SEQ ID NO: 73).

FIG. 11 illustrates workflows for creating gene edited plants. Panel A shows a Fast-TrACC method in which developmental regulators and hormone biosynthesis genes are delivered by Agrobacterium in various combinations to seedlings germinated in liquid medium³². Panel B shows a method for inducing gene edited shoots on soil-grown plants.

FIG. 12 is a graph showing production of casbene in tomato fruit over different stages of fruit development wherein DbTPS1 (Daphne genkwa terpene synthase 1; SEQ ID NO: 74) is expressed in the tomato fruit tissue.

FIG. 13 is a graph showing production of 11-hydroxyvulgarisane in tomato fruit over different stages of fruit development wherein PvHVS (Prunella vulgaris hydroxyvulgarisane synthase; SEQ ID NO: 73) is expressed in the tomato fruit tissue.

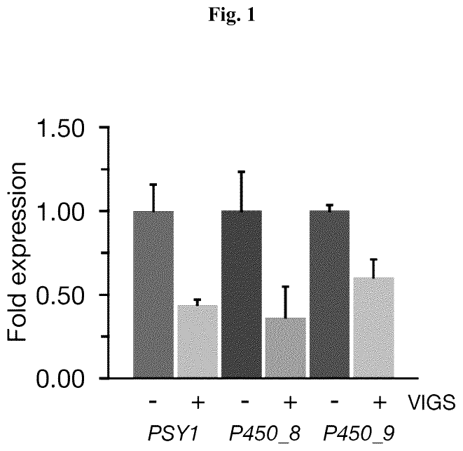

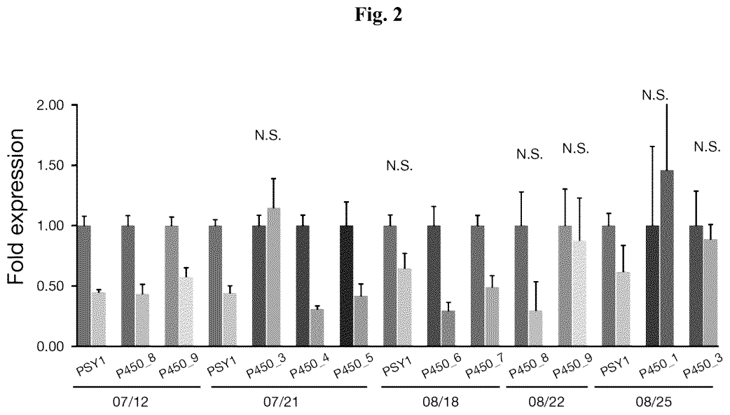

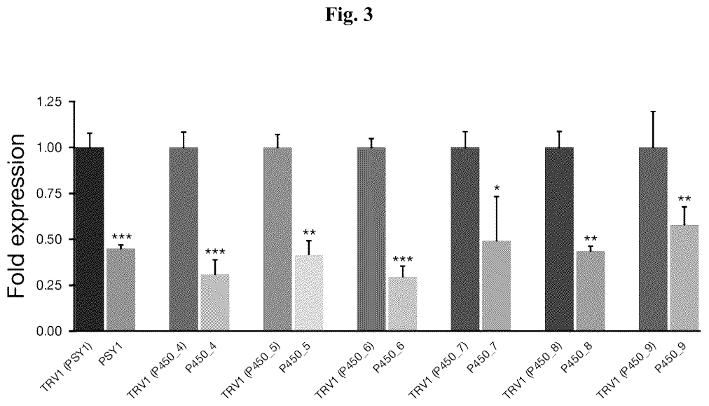

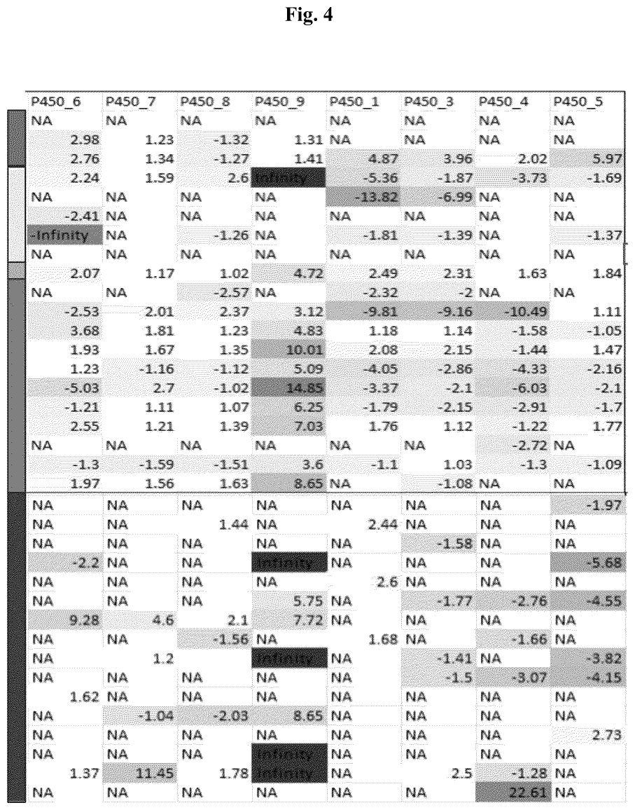

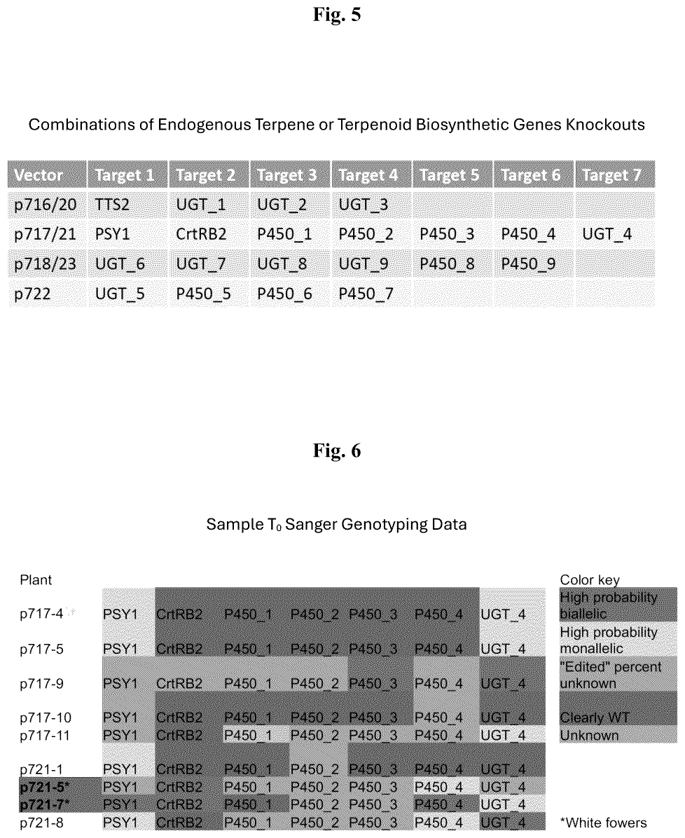

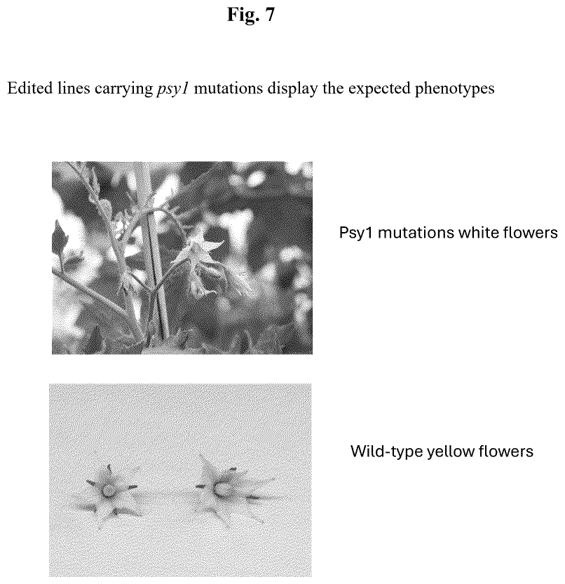

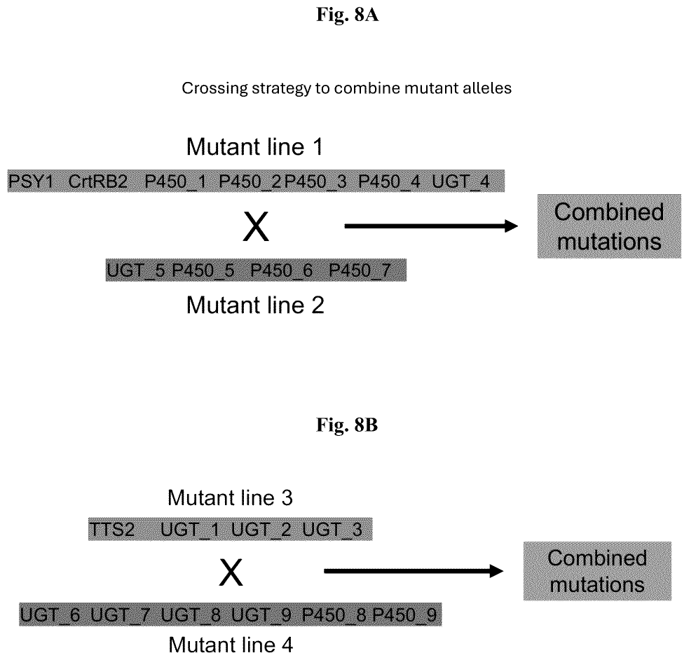

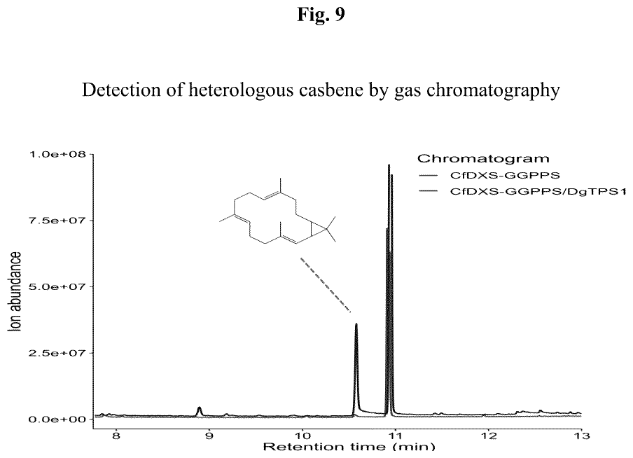

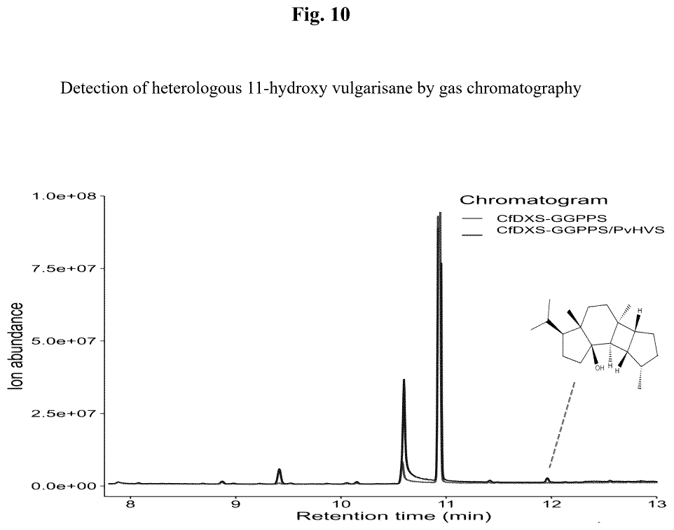

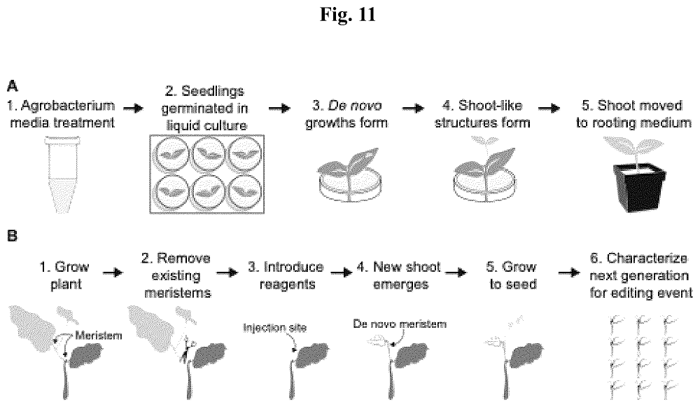

### DETAILED DESCRIPTION

In certain embodiments, the present disclosure provides modified tomato plants (Solanum lycopersicum) engineered to biosynthesize minimal terpenoids which serves as a chassis for expression of high value terpenoids. The modified tomato plants comprise at least one targeted modification in at least one endogenous terpene or terpenoid biosynthetic gene resulting in decreased expression of the endogenous terpene or terpenoid biosynthetic gene relative to a reference plant lacking the modification. The targeted modification in the endogenous terpene or terpenoid biosynthetic gene comprises an insertion, replacement, and/or deletion of one or more nucleotides in the endogenous terpene or terpenoid biosynthetic. Decreased expression in the endogenous terpene or terpenoid biosynthetic genes results in a depletion of carotene in fruit of the tomato relative to the reference tomato plant lacking the modification. Heterologous terpene or terpenoid biosynthetic genes can be introduced into the modified tomato plant for synthesizing high value terpenoids, such as Nootkatone, Viridiflorol and Santalol (C₁₅), Neoclerodane, Carnosol and Leubethanol (C₂₀), Squalene and Betulinic acid (C₃₀), and Astaxanthin (C₄₀) across four classes of terpenoids.

This disclosure will be better understood in view of the following definitions, which are provided for clarification and are not intended to limit the scope of the subject matter that is disclosed herein.

### Definitions

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as well as necessarily defines the exact complements, as is known to one of ordinary skill in the art. Where a term is provided in the singular, the inventors also contemplate aspects of the disclosure described by the plural of that term.

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “CRISPR” refers to Clustered Regularly Interspaced Short Palindromic Repeats. CRISPR and CRISPR-associated (Cas) genes, are collectively referred to as CRISPR-Cas or CRISPR/Cas systems, which are adaptive immune systems in archaea and bacteria that defend particular species against foreign genetic elements.

As used herein, the terms “RNA guide,” “gRNA,” or “RNA guide sequence” refer to any RNA molecule that facilitates the targeting of a polypeptide described herein to a target nucleic acid. For example, an RNA guide can be a molecule that recognizes (e.g., binds to) a target nucleic acid. An RNA guide may be designed to be complementary to a specific nucleic acid sequence. An RNA guide comprises a DNA targeting sequence (also referred to herein as a spacer sequence), and a crRNA sequence (also referred to as a direct repeat (DR) sequence) that facilitates binding of the RNA guide to a Cas enzyme.

By “polynucleotide” is meant a nucleic acid molecule containing multiple nucleotides and refers to “oligonucleotides” (defined here as a polynucleotide molecule of between 2-25 nucleotides in length) and polynucleotides of 26 or more nucleotides. Polynucleotides are generally described as single- or double-stranded. Where a polynucleotide contains double-stranded regions formed by intra- or intermolecular hybridization, the length of each double-stranded region is conveniently described in terms of the number of base pairs. Aspects of this disclosure include the use of polynucleotides or compositions containing polynucleotides; embodiments include one or more oligonucleotides or polynucleotides or a mixture of both, including single- or double-stranded RNA or single- or double-stranded DNA or single- or double-stranded DNA/RNA hybrids or chemically modified analogues or a mixture thereof. In various embodiments, a polynucleotide (such as a single-stranded DNA/RNA hybrid or a double-stranded DNA/RNA hybrid) includes a combination of ribonucleotides and deoxyribonucleotides (e.g., synthetic polynucleotides consisting mainly of ribonucleotides but with one or more terminal deoxyribonucleotides or synthetic polynucleotides consisting mainly of deoxyribonucleotides but with one or more terminal dideoxyribonucleotides), or includes non-canonical nucleotides such as inosine, thiouridine, or pseudouridine. In embodiments, the polynucleotide includes chemically modified nucleotides (see, e.g., Verma and Eckstein (1998) Annu. Rev. Biochem., 67:99-134); for example, the naturally occurring phosphodiester backbone of an oligonucleotide or polynucleotide can be partially or completely modified with phosphorothioate, phosphorodithioate, or methylphosphonate internucleotide linkage modifications; modified nucleoside bases or modified sugars can be used in oligonucleotide or polynucleotide synthesis; and oligonucleotides or polynucleotides can be labelled with a fluorescent moiety (e.g., fluorescein or rhodamine or a fluorescence resonance energy transfer or FRET pair of chromophore labels) or other label (e.g., biotin or an isotope). Modified nucleic acids, particularly modified RNAs, are disclosed in U.S. Pat. No. 9,464,124, incorporated by reference in its entirety herein. For some polynucleotides (especially relatively short polynucleotides, e.g., oligonucleotides of 2-25 nucleotides or base-pairs, or polynucleotides of about 25 to about 300 nucleotides or base-pairs), use of modified nucleic acids, such as locked nucleic acids (“LNAs”), is useful to modify physical characteristics such as increased melting temperature (Tₘ) of a polynucleotide duplex incorporating DNA or RNA molecules that contain one or more LNAs; see, e.g., You et al. (2006) Nucleic Acids Res., 34:1-11 (e60), doi: 10.1093/nar/gk1175.

In the context of the genome targeting methods described herein, the phrase “contacting a genome” with an agent means that an agent responsible for effecting the targeted genome modification (e.g., a break, a deletion, a rearrangement, or an insertion) is delivered to the interior of the cell so the directed mutagenic action can take place.

In the context of discussing or describing the ploidy of a plant cell, the “n” (as in “a ploidy of 2n”) refers to the number of homologous pairs of chromosomes and is typically equal to the number of homologous pairs of gene loci on all chromosomes present in the cell.

The term “inbred variety” refers to a genetically homozygous or substantially homozygous population of plants that preferably comprises homozygous alleles at about 95%, preferably 98.5% or more of its loci. An inbred line can be developed through inbreeding (i.e., several cycles of selfing, more preferably at least 5, 6, 7 or more cycles of selfing) or doubled haploidy resulting in a plant line with a high uniformity. Inbred lines breed true, e.g., for one or more or all phenotypic traits of interest. An “inbred”, “inbred individual, or “inbred progeny” is an individual sampled from an inbred line.

“F1, F2, F3, etc.” refers to the consecutive related generations following a cross between two parent plants or parent lines. The plants grown from the seeds produced by crossing two plants or lines is called the F1 generation. Selfing the F1 plants results in the F2 generation, etc. “F1 hybrid” plant (or F1 hybrid seed) is the generation obtained from crossing two inbred parent lines. Thus, F1 hybrid seeds are seeds from which F1 hybrid plants grow. F1 hybrids are more vigorous and higher yielding, due to heterosis.

Hybrid seed: Hybrid seed is seed produced by crossing two different inbred lines (i.e. a female inbred line with a male inbred). Hybrid seed is heterozygous over a majority of its alleles.

As used herein, the term “variety” refers to a group of similar plants that by structural or genetic features and/or performance can be distinguished from other varieties within the same species.

The term “cultivar” (for cultivated variety) is used herein to denote a variety that is not normally found in nature but that has been created by humans, i.e., having a biological status other than a “wild” status, which “wild” status indicates the original non-cultivated, or natural state of a plant or accession. The term “cultivar” includes, but is not limited to, semi-natural, semi-wild, weedy, traditional cultivar, landrace, breeding material, research material, breeder's line, synthetic population, hybrid, founder stock/base population, inbred line (parent of hybrid cultivar), segregating population, mutant/genetic stock, and advanced/improved cultivar. The term “elite background” is used herein to indicate the genetic context or environment of a targeted mutation of insertion.

The term “allele(s)” means any of one or more alternative forms of a gene at a particular locus, all of which alleles relate to one trait or characteristic at a specific locus. In a diploid cell of an organism, alleles of a given gene are located at a specific location, or locus (loci plural), on a chromosome. One allele is present on each chromosome of the pair of homologous chromosomes. A diploid plant species may comprise a large number of different alleles at a particular locus. These may be identical alleles of the gene (homozygous) or two different alleles (heterozygous).

The phrase “allelic variant” as used herein refers to a polynucleotide or polypeptide sequence variant found in different alleles of a given gene. Polynucleotide sequence variants of such allelic variants can occur in coding and/or non-coding regions of the gene.

The term “locus” (loci plural) means a specific place or places or a site on a chromosome where for example a QTL, a gene or genetic marker is found.

As used herein, the term “endogenous gene” or “endogenous nucleic acid” refers to a nucleic acid that is normally found in and/or produced by a given plant or cell in nature. An “endogenous gene” or “endogenous nucleic acid” is also referred to as a “native nucleic acid” or a nucleic acid that is “native” to a given plant or cell.

As used herein, the term “heterologous” when used in reference to a nucleic acid or protein refers to a nucleic acid or protein that has been manipulated in some way. For example, a heterologous nucleic acid includes a nucleic acid from one species introduced into another species. A heterologous nucleic acid also includes a nucleic acid that is native to an organism that has been altered in some way (e.g., mutated, added in multiple copies, present in a locus within the genome, expressed from an autonomously replicating vector, linked to a non-native promoter, linked to a mutated promoter, or linked to an enhancer sequence, etc.). Heterologous nucleic acids may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript). In some cases, heterologous nucleic acids are distinguished from endogenous plant genes in that the heterologous nucleic acids can be joined to nucleotide sequences comprising regulatory elements such as promoters that are not found naturally associated with the nucleic acid. In another example, the heterologous nucleic acids are associated with portions of the chromosome not found in nature (e.g., genes expressed in loci where the gene is not normally expressed).

The terms “identical” or percent “identity”, as used herein, in the context of two or more nucleic acids or polypeptide sequences, refer to two or more sequences or subsequences that are the same or have a specified percentage of amino acid residues or nucleotides that are the same (e.g., 75% identity, 80% identity, 85% identity, 90% identity, 95% identity, 97% identity, 98% identity, 99% identity, or 100% identity in pairwise comparison). Sequence identity can be determined by comparison and/or alignment of sequences for maximum correspondence over a comparison window, or over a designated region as measured using a sequence comparison algorithm, or by manual alignment and visual inspection. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. A “reference sequence” is a defined sequence used as a basis for a sequence comparison; a reference sequence may be a subset of a larger sequence.

As used herein, a “native” nucleic acid or polypeptide means a DNA, RNA or amino acid sequence or segment that has not been manipulated in vitro, i.e., has not been isolated, purified, and/or amplified.

As used herein, the term “wild-type” when made in reference to a gene refers to a functional gene common throughout an outbred population. As used herein, the term “wild-type” when made in reference to a gene product refers to a functional gene product common throughout an outbred population. A functional wild-type gene is that which is most frequently observed in a population and is thus arbitrarily designated the “normal” or “wild-type” form of the gene. As used herein, the term “wild-type” when made in reference to a plant refers to the plant type common throughout an outbred population that has not been genetically manipulated to contain an expression cassette, e.g., any of the expression cassettes described herein.

As used herein, the phrase “biological sample” refers to either intact or non-intact (e.g. milled seed or plant tissue, chopped plant tissue, lyophilized tissue) plant tissue. It may also be an extract comprising intact or non-intact seed or plant tissue. The biological sample can comprise flour, meal, syrup, oil, starch, and cereals manufactured in whole or in part to contain crop plant by-products. In certain embodiments, the biological sample is “non-regenerable” (i.e., incapable of being regenerated into a plant or plant part). In certain embodiments, the biological sample refers to a homogenate, an extract, or any fraction thereof containing genomic DNA of the organism from which the biological sample was obtained, wherein the biological sample does not comprising living cells.

As used herein, the terms “correspond,” “corresponding,” and the like, when used in the context of an nucleotide position, mutation, and/or substitution in any given polynucleotide (e.g., an allelic variant of a CrtR-B2 gene of SEQ ID NO: 2 or 24) with respect to the reference polynucleotide sequence (e.g., SEQ ID NO: 2 or 24) all refer to the position of the polynucleotide residue in the given sequence that has identity to the residue in the reference nucleotide sequence when the given polynucleotide is aligned to the reference polynucleotide sequence using a pairwise alignment algorithm (e.g., CLUSTAL O 1.2.4 with default parameters).

The phrase “operably linked” refers to a juxtaposition wherein the components so described are in a relationship permitting them to function in their intended manner. For instance, a promoter is operably linked to a coding sequence if the promoter affects its transcription or expression.

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, silk, flowers, kernels, ears, cobs, husks, or stalks. In contrast, some plant cells are not capable of being regenerated to produce plants and are referred to herein as “non-regenerable” plant cells.

As used herein, terms “replace,” “replacement,” “replacing” and the like are used synonymously with the terms “substitute,” “substitution,” “substituting,” and the line with regards to changes in nucleotide residues in a polynucleotide molecule.

The term “isolated” as used herein means having been removed from its natural environment.

As used herein, the terms “include,” “includes,” and “including” are to be construed as at least having the features to which they refer while not excluding any additional unspecified features.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Tomato Chassis

The present disclosure describes the modification of endogenous terpene or terpenoid biosynthetic genes in tomato plants to reduce their expression and thereby minimize the terpenoids produced in the modified tomato plant which then serves as a chassis for expression of high value heterologous terpenoids. The modified tomato plants comprise at least one targeted modification in at least one endogenous terpene or terpenoid biosynthetic gene resulting in decreased expression of the endogenous terpene or terpenoid biosynthetic gene relative to a reference plant lacking the modification. In various embodiments, the modification to the endogenous terpene or terpenoid biosynthetic genesis not naturally occurring and/or is not exclusively obtained through an essentially biological process. For example, endogenous terpene or terpenoid biosynthetic genes that can be modified by insertion, replacement, and/or deletion of one or more nucleotides is provided in Table 1.

TABLE 1
Endogenous terpene or terpenoid biosynthetic genes.

| | Target Tomato | | | |
|---|---|---|---|---|
| Gene ID from | Endogenous terpene | | Gene | Amino Acid |
| M82 Tomato | or terpenoid | Gene | Sequence | Sequence |
| genome | biosynthetic gene | SEQ ID NO: | Description | SEQ ID NO: |
| Solly.M82.03G005440 | Phytoene synthase | 1 | DNA genomic | 45 |
| | (Psy1) | 23 | sequence | |
| | | | Coding DNA | |
| | | | sequence | |
| Solly.M82.03G003380 | Beta-carotene | 2 | DNA genomic | 46 |
| | hydroxylase | 24 | sequence | |
| | (CrtR-B2) | | Coding DNA | |
| | | | sequence | |
| Solly.M82.12G001650.1 | Triterpene synthase -1 | 3 | DNA genomic | 47 |
| | (TTS1) | 25 | sequence | |
| | | | Coding DNA | |
| | | | sequence | |
| Solly.M82.12G001650.2 | Triterpene synthase -2 | 4 | DNA genomic | 48 |
| | (TTS2) | 26 | sequence | |
| | | | Coding DNA | |
| | | | sequence | |
| Solly.M82.03G020240 | Cytochrome P450_1 | 5 | DNA genomic | 49 |
| | (P450_1) | 27 | sequence | |
| | | | Coding DNA | |
| | | | sequence | |
| Solly.M82.03G023730 | Cytochrome P450_2 | 6 | DNA genomic | 50 |
| | (P450_2) | 28 | sequence | |
| | | | Coding DNA | |
| | | | sequence | |
| Solly.M82.04G014720 | Cytochrome P450_3 | 7 | DNA genomic | 51 |
| | (P450_3) | 29 | sequence | |
| | | | Coding DNA | |
| | | | sequence | |
| Solly.M82.04G020500 | Cytochrome P450_4 | 8 | DNA genomic | 52 |
| | (P450_4) | 30 | sequence | |
| | | | Coding DNA | |
| | | | sequence | |
| Solly.M82.07G023410 | Cytochrome P450_5 | 9 | DNA genomic | 53 |
| | (P450_5) | 31 | sequence | |
| | | | Coding DNA | |
| | | | sequence | |
| Solly.M82.07G025510 | Cytochrome P450_6 | 10 | DNA genomic | 54 |
| | (P450_6) | 32 | sequence | |
| | | | Coding DNA | |
| | | | sequence | |
| Solly.M82.07G025530 | Cytochrome P450_7 | 11 | DNA genomic | 55 |
| | (P450_7) | 33 | sequence | |
| | | | Coding DNA | |
| | | | sequence | |
| Solly.M82.09G018300 | Cytochrome P450_8 | 12 | DNA genomic | 56 |
| | (P450_8) | 34 | sequence | |
| | | | Coding DNA | |
| | | | sequence | |
| Solly.M82.10G023540 | Cytochrome P450_9 | 13 | DNA genomic | 57 |
| | (P450_9) | 35 | sequence | |
| | | | Coding DNA | |
| | | | sequence | |
| Solly.M82.01G038680 | Uracil dependent | 14 | DNA genomic | 58 |
| | glucosyl transferase -1 | 36 | sequence | |
| | (UGT_1) | | Coding DNA | |
| | | | sequence | |
| Solly.M82.01G038690 | Uracil dependent | 15 | DNA genomic | 59 |
| | glucosyl transferase -2 | 37 | sequence | |
| | (UGT_2) | | Coding DNA | |
| | | | sequence | |
| Solly.M82.02G014810 | Uracil dependent | 16 | DNA genomic | 60 |
| | glucosyl transferase -3 | 38 | sequence | |
| | (UGT_3) | | Coding DNA | |
| | | | sequence | |
| Solly.M82.03G026450 | Uracil dependent | 17 | DNA genomic | 61 |
| | glucosyl transferase -4 | 39 | sequence | |
| | (UGT_4) | | Coding DNA | |
| | | | sequence | |
| Solly.M82.08G001390 | Uracil dependent | 18 | DNA genomic | 62 |
| | glucosyl transferase -5 | 40 | sequence | |
| | (UGT_5) | | Coding DNA | |
| | | | sequence | |
| Solly.M82.09G023010 | Uracil dependent | 19 | DNA genomic | 63 |
| | glucosyl transferase -6 | 41 | sequence | |
| | (UGT_6) | | Coding DNA | |
| | | | sequence | |
| Solly.M82.09G025760 | Uracil dependent | 20 | DNA genomic | 64 |
| | glucosyl transferase -7 | 42 | sequence | |
| | (UGT_7) | | Coding DNA | |
| | | | sequence | |
| Solly.M82.10G025440 | Uracil dependent | 21 | DNA genomic | 65 |
| | glucosyl transferase -8 | 43 | sequence | |
| | (UGT_8) | | Coding DNA | |
| | | | sequence | |
| Solly.M82.10G026010 | Uracil dependent | 22 | DNA genomic | 66 |
| | glucosyl transferase -9 | 44 | sequence | |
| | (UGT_9) | | Coding DNA | |
| | | | sequence | |

In various embodiments, the endogenous terpene or terpenoid biosynthetic genes that are modified to decreased expression comprise: (a) a beta carotene hydroxylase, a phytoene synthase, a cytochrome P450, a uracil dependent glucosyl transferase, or a combination thereof, (b) a DNA molecule having at least 90%, at least 95%, at least 96%, at least 97%, at least 98%, at least 99%, or 100% sequence identity to the nucleic acid sequence set forth in any one of SEQ ID NOs: 1-44 or an allelic variant thereof, (c) a beta carotene hydroxylase (CrtR-B2; SEQ ID NO: 2 or 24), phytoene synthase (PSY1; SEQ ID NO: 1 or 23), cytochrome P450_1 (P450_1; SEQ ID NO: 5 or 27), cytochrome P450_2 (P450_2; SEQ ID NO: 6 or 28), cytochrome P450_3 (P450_3; SEQ ID NO: 7 or 29), cytochrome P450_4 (P450_4; SEQ ID NO: 8 or 30), and uracil dependent glucosyl transferase-4 (UGT_4; SEQ ID NO: 17 or 39), or (d) uracil dependent glucosyl transferase-6 (UGT_6; SEQ ID NO: 19 or 41), uracil dependent glucosyl transferase-7 (UGT_7; SEQ ID NO: 20 or 42), uracil dependent glucosyl transferase-8 (UGT_8; SEQ ID NO: 21 or 43), uracil dependent glucosyl transferase-9 (UGT_9; SEQ ID NO: 22 or 44), cytochrome P450_8 (P450_8; SEQ ID NO: 12 or 34), and cytochrome P450_9 (P450_9; SEQ ID NO: 13 or 35).

### Heterologous Terpene or Terpenoid Biosynthetic Genes

The modified tomato plant comprising a modification in at least one endogenous terpene or terpenoid biosynthetic gene can be further modified by introducing a heterologous terpene or terpenoid biosynthetic gene. The heterologous terpene or terpenoid biosynthetic gene can encode a terpene or terpenoid biosynthetic protein that produces high value terpenoid compounds, such as those provided in Table 2.

TABLE 2
Examples of high value terpenoid compounds. Class: C₁₅,
sesqui-; C₂₀, di-; C₃₀, tri-; C₄₀, tetraterpene (carotenoid).

| Class | Target | Current source | Use | Value |
|---|---|---|---|---|
| C₁₅ | Nootkatone* | Grapefruit | Mosquito, tick repellant, flavor | $60/g |
| | | | compound | |
| C₁₅ | Santalol* | Sandalwood | Fragrance industry, mosquitocidal | $290/g |
| C₁₅ | Viridiflorol* | Broad-leaved | Fragrance industry | $7000/g |
| | | paperbark, tea | | |
| | | tree | | |
| C₂₀ | Carnosol*/ | Rosemary, Salvia | Anti-oxidant/ antimicrobial; meat | $3520/g |
| | Tanshinones | miltiorrhiza | preservative | |
| C₂₀ | Leubethanol* | Texas sage | Anti-microbial activity (TB) | NA |
| C₂₀ | Neoclerodane* | Ajuga reptans, | Agrichemical insect deterrent | NA |
| | | related | | |
| | | Ajugoideae | | |
| C₂₀ | Resin acids | Conifers | Constituents of high-end inks, glues, | NA |
| | | | coatings | |
| C₂₀ | Steviol | Stevia | Zero-calorie sweetener | NA |
| | glycosides | | | |
| C₃₀ | Squalene* | Shark liver oil, | Cosmetics, personal care | $35/kg |
| | | olive oil | | |
| C₃₀ | Ambroxoides | (Sperm whale) | Fragrance industry | $23/g |
| | | Salvia sclarea | | |
| C₃₀ | Betulinic | White birch | Antiretroviral, antimalarial, under | $9500/g |
| | acid* | | investigation for cancer treatment | |
| C₃₀ | Mogrosides | Monk fruit | Zero-calorie sweetener | NA |
| C₄₀ | Astaxanthin* | Haem. pluvialis, | Antioxidant, food colorant | $5/g |
| | | synthetic | | |

### Terpene Biosynthetic Pathway.

Despite their enormous structural diversity, terpene biosynthetic pathways in plants follow simple, modular steps. Terpene biosynthesis is highly compartmentalized, with two independent routes to the universal C5 building blocks isopentyl diphosphate (IDP) and dimethylallyl diphosphate (DMAPP) localized to the cytosol or plastid. This is reflected in the subcellular organization of the routes to C₁₀ up to C₄₀ terpenoids: scaffolds of mono-, di- and carotenoids are formed in the plastid, while sesqui- and triterpenoids originate in the cytosol. Native terpenoids in tomato are localized to dedicated tissues or organs, with the smaller mono- to diterpenoids accumulating predominantly in the leaf and trichomes. Despite highly active mevalonate (MVA) and methylerythritol 4-phosphate (MEP) pathways during fruit growth and ripening, tomato fruits produce only minute amounts of monoterpenes and no detectable sesquiterpenes. Practically, the entire carbon flux of the terpenoid pathway is routed into triterpenes and tetra-terpene (carotenoid) in the maturing fruit. Taking advantage of the native organization of these pathways is described herein for the fruit-specific engineering of novel terpene bioproducts. Thus, the modification of endogenous tomato terpene or terpenoid biosynthetic genes to decrease their expression and expression of heterologous terpene or terpenoid biosynthetic genes focuses on production of specialized, non-essential tomato tri- and tetraterpenes accumulating in the fruit and the waxy cuticle of the fruit epidermis.

Terpene synthases (TPS), encoded by medium-sized gene families in plants, typically catalyze cyclization of the universal linear precursors geranyl diphosphate (GDP, C₁₀), farnesyl diphosphate (FDP, C₁₅) and geranylgeranyl diphosphate (GGDP, C₂₀) to yield products for general (i.e., hormones, sterols) and specialized metabolism. While GGDP is the nearly universal precursor for diterpenes across all kingdoms of life, an unusual biosynthesis route exists in Solanaceae and the taxonomically more distant figwort families (Scrophulaceae) via nerylneryl diphosphate (C20, NNDP) to unusual cis-prenyl diterpenes. In tomato, the TPS complement is known, with all genes classified into mono-, sesqui-, di-, tri-, and tetra-terpene (carotenoid) metabolism.

The highly localized production of specialized terpenoids in tomato is mirrored by their gene expression (Table 3).

TABLE 3
Summary of endognous tomato terpene synthases (TPS).

| | | | Predominantly |
|---|---|---|---|
| | | Specialized | expressed |
| Class | Total | metabolism | tissue |
| C₁₀ (mono-) | 8 | 8 | young leaf, trichomes |
| C₁₅ (sesqui-) | 19 | 19 | young leaf, trichomes |
| C₂₀ (di-) | 6 | 4 | petioles/flower/leaf |
| C₃₀(mono-) | 3 | 2 targets* | fruit epidermis |
| C₄₀ (carotenoid/ | 2 | 1 target* | fruit |
| tetraterpemoid) | | | |

*Fruit TPS to be targeted in this project.

Transcripts for a large number of endogenous TPS forming volatiles accumulate in glandular trichomes of young leaves, stems and early developmental stages of the fruit which are lost during harvest. In contrast, those expressed in the fruit and fruit cuticle are limited to tri- and tetraterpenes. This fruit-specific set of terpene synthases responsible for formation of tri- and tetraterpene specialized metabolites is highly limited and non-essential. Distinct from essential sterols, in tomato fruits the route to triterpenoid specialized metabolites, comprising approximately a fourth of the mass in the wax, is formed during early fruit expansion. Similarly, characteristic for the ripening process of tomato fruits is a substantial formation of carotenoids, i.e., tetraterpene pigments, which is distinct from biosynthesis for photosynthesis in leaves. During fruit ripening, tomato expresses discrete, separate sets of genes encoding the rate-limiting steps. Geranylgeranyl diphosphate synthase, phytoene synthase (Psy), lycopene β-cyclase, and β-carotene hydroxylase are all encoded by duplicate isoforms constitutively expressed in leaves, or specific for chromoplasts in flowers and/or fruits, respectively. For example, a viable knock-out of in Psy1 lacks most of the carotenoids in fruits but not in leaves, while inactivation of Cyc-B gene results in accumulation of low levels of the red pigment lycopene in the flowers and removes β-carotene in fruits without affecting carotenoids in leaves. Accumulation of carotenoids in tomato fruits can reach 4.3 g/kg dry weight.

### Cytochromes P450 (P450s)

The irreversible nature of reactions catalyzed by P450s makes these enzymes landmarks in the evolution of plant metabolic pathways. Founding members of P450 families are often associated with general metabolic pathways, restricted to single copy or very few representatives, indicative of purifying selection. Recruitment of those and subsequent expansions into multi-member gene families generates the genetic basis for functional diversification, which is characteristic of specialized metabolism. This is recognized by the emergence of fast evolving subfamilies with multiple members. The key clan for plant specialized metabolism, with major expansions, is the CYP71 clan, containing over half of all P450s and coined the cradle of terpene diversity. Thirty-seven subfamilies were shown to be involved in terpenoid specialized metabolism; one catalyzes formation of ent-kaurenoic acid, a critical precursor to the plant phytohormone gibberellic acid.

### UDP-dependent Glycosyl Transferases (UGTs)

Glycosyl transferases (GTs) are a universal group of enzymes catalyzing the transfer of a sugar moiety from an activated donor molecule onto other sugars or small molecule acceptors yielding poly-, di-glycoside, and various derivatives of specialized metabolism. Where understood, the products are of biological relevance for functions in adaptation and interaction with the environment and their biosynthesis may involve the action of hundreds of more or less promiscuous GTs. GTs that typically transfer sugars onto small molecules fall into GT family. In plants, GTs utilize UDP-activated sugars as the major donor molecule. They contain the well conserved 44-amino acid Plant Secondary Product Glycosyltransferase (PSPG) motif close to the C-terminus as a universal feature representing the nucleotide-diphosphate-sugar binding site of the enzymes. UGTs involved in plant specialized metabolism often display substrate promiscuity, with heterologously expressed enzymes recognizing a broad spectrum of acceptor molecules. This promiscuity plausibly contributes to the enormous chemical variation of small molecules and high potential to non-specifically modify engineered pathways of target products in tomato.

Plants can be used as a chassis for installing specialized terpene pathways; however, there are inherent challenges, as pools of C5 building blocks are limited and there is a tendency for conversion of the activated products into conjugates or other derivatives via endogenous cytochrome P450s and glycosyl transferases. The introduced, non-native pathways will therefore compete with native routes. Also, the accumulation of mixtures of related, yet non-desired terpenes, complicates purification of engineered bioproducts. Last, due to the activity of endogenous unspecific enzymes, desired bioproducts undergo further conversion into derivatives and conjugates. Indeed, previous studies in engineered members of the Solanaceae focused on production of mono-sesqui, or di-terpene alcohols or carboxylates and detected derivatives generated through non-specific endogenous activities; considerable amounts of the target products accumulate as complex sugar conjugates or as acetylated derivatives. For example, in tobacco plants stably transformed with geraniol synthase from Valeriana officinalis, a total of 19 unwanted derivatives were produced, including mono-, di- and tri-glycosides. Production of derivatives was also seen in engineering of the sesquiterpene artemisinic acid pathway, diterpene resin acid production, and triterpene synthases coupled with a cytochrome P450s.

With distinct routes to specialized terpenoids, insulated from essential general metabolism and dedicated anatomies for product sequestration (hydrophobic epidermal cuticular waxes; fruit chromoplasts), tomato fruits therefore are a useful chassis for rational engineering of terpenoid metabolism. Reducing expression of native pathways free-up large pools of available precursor material for re-routing into engineered, non-native pathways, with capacities for sequestration and stabilization of high-value products in dedicated and specialized tissues. To address challenges in synthesis of unwanted derivatives and conjugates, native tri- and tetra-TPSs expressed in fruit are removed along with removal of co-expressed fruit-specific P450s and UGTs that could react with engineered terpene products in the target chassis.

TABLE 4
Examples of Heterologous terpene or terpenoid biosynthetic genes and pathway products.

| | | | | First | |
|---|---|---|---|---|---|
| | | | | Pathway | |
| | | | | Precursor | First Pathway |
| Pathway Product | | First Pathway | Second Pathway | Boosting | Precursor |
| Name | Pathway Product Structure | Specific Gene | Specific Gene | Gene | Boosting Gene |
| trans-abienol | 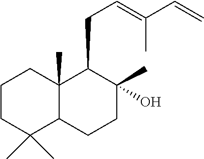 [SMILES: C=C/C(C)=C/C[C@@H]1[C@@]2(C)CCCC(C)(C)C2CC[C@@]1(C)O] | Plectranthus barbatus (syn. Coleus forskohlii) Terpene Synthase 2 (CfTPS2; SEQ ID NO: 67) ² | Origanum majorana Terpene Synthase 3 (OmTPS3; SEQ ID NO: 68) | Plectranthus barbatus (syn. Coleus forskohlii) 1-deoxy-D- xylulose-5- phosphate synthase (CfDXS; SEQ ID NO: 69) | Plectranthus barbatus (syn. Coleus forskohlii) Geranylgeranyl pyrophosphate synthase (CfGPPS; SEQ ID NO: 70) ⁷ |
| casbene | 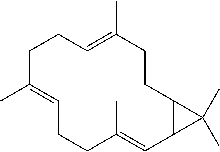 | Daphne genkwa Terpene Synthase 1 (DgTPS1; SEQ ID NO: 74) | Not applicable | Plectranthus barbatus (syn. Coleus forskohlii) 1-deoxy-D- xylulose-5- phosphate synthase (CfDXS; SEQ ID NO: 69) ⁷ | Plectranthus barbatus (syn. Coleus forskohlii) Geranylgeranyl pyrophosphate synthase (CfGPPS SEQ ID NO: 70) ⁷ |
| 5- hydroxycasbene | 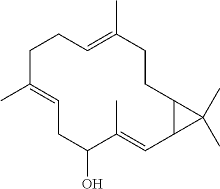 | Daphne genkwa Terpene Synthase 1 (DgTPS1; SEQ ID NO: 74) | Euphorbia lathyris cytochrome P450 CYP726A27 (EICYP726A27; SEQ ID NO: 75) | Plectranthus barbatus (syn. Coleus forskohlii) 1-deoxy-D- xylulose-5- phosphate synthase (CfDXS; SEQ ID NO: 69) ⁷ | Plectranthus barbatus (syn. Coleus forskohlii) Geranylgeranyl pyrophosphate synthase (CfGPPS; SEQ ID NO: 70) ⁷ |
| intermedeol | 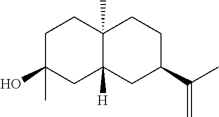 [SMILES: C=C(C)[C@@H]1CC[C@]2(C)CC[C@](C)(O)C[C@H]2C1] | Plastidial targeted Sorghum bicolor Terpene Synthase 10 (TP-SbTPS10; SEQ ID NO: 72) | Not applicable | Plectranthus barbatus (syn. Coleus forskohlii) 1-deoxy-D- xylulose-5- phosphate synthase (CfDXS; SEQ ID NO: 69) ⁷ | Plastidial targeted Arabidopsis thaliana Farnesyl pyrophosphate synthase (TP-AtFPPS; SEQ ID NO: 71) ⁸ |
| 11- hydroxyvulgarisane | 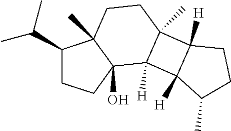 [SMILES: CC(C)[C@@H]1CC[C@@]2(O)[C@@H]3[C@@H]4[C@@H](CC[C@@H]4C)[C@]3(C)CC[C@@]12C] | Prunella vulgaris 11- hydroxyvulgarisane synthase (PvHVS; SEQ ID NO: 73) ⁶ | Not applicable | Plectranthus barbatus (syn. Coleus forskohlii) 1-deoxy-D- xylulose-5- phosphate synthase (CfDXS; SEQ ID NO: 69) ⁷ | Plectranthus barbatus (syn. Coleus forskohlii) Geranylgeranyl pyrophosphate synthase (CfGPPS; SEQ ID NO: 70) ⁷ |

TABLE 5
Examples of heterologous terpene or terpenoid biosynthetic genes
for insertion into tomato terpene or terpenoid depleted chassis.

| Heterologous | | Gene | Amino Acid |
|---|---|---|---|
| terpene or terpenoid | Gene | Sequence | Sequence |
| biosynthetic gene | Abbreviation | SEQ ID NO: | SEQ ID NO: |
| Plectranthus barbatus | CfTPS2 | 67 | 76 |
| (syn. Coleus forskohlii) | | | |
| Terpene Synthase 2 | | | |
| Origanum majorana | OmTPS3 | 68 | 77 |
| Terpene Synthase 3 | | | |
| Plectranthus barbatus | CfDXS | 69 | 78 |
| (syn. Coleus forskohlii) | | | |
| 1-deoxy-D-xylulose-5- | | | |
| phosphate synthase | | | |
| Plectranthus barbatus | CfGGPPS | 70 | 79 |
| (syn. Coleus forskohlii) | | | |
| Geranylgeranyl | | | |
| pyrophosphate synthase | | | |
| Plastidial targeted | TP-AtFPPS | 71 | 80 |
| Arabidopsis thaliana | | | |
| Farnesyl pyrophosphate | | | |
| synthase | | | |
| Plastidial targeted | TP-SbTPS10 | 72 | 81 |
| Sorghum bicolor Terpene | | | |
| Synthase 10 | | | |
| Prunella vulgaris 11- | PvHVS | 73 | 82 |
| hydroxyvulgarisane | | | |
| synthase | | | |
| Daphne genkwa | DgTPS1 | 74 | 83 |
| Terpene Synthase 1 | | | |
| Euphorbia lathyris | ElCYP726A27 | 75 | 84 |
| cytochrome P450 | | | |

Using CRISPR/Cas methods as described below, various modifications to genomic endogenous terpene or terpenoid biosynthetic genes were made to make tomato chassis depleted for tri-(C30), and tetra (C40)-terpenes in tomato fruit. One of the tomato chassis was developed to have modifications in the following endogenous terpene or terpenoid biosynthetic genes to reduce their expression (e.g. knockouts): beta carotene hydroxylase (CrtR-B2; SEQ ID NO: 2), phytoene synthase (PSY1; SEQ ID NO: 1), cytochrome P450_1 (P450_1; SEQ ID NO: 5), cytochrome P450_2 (P450_2; SEQ ID NO: 6), cytochrome P450_3 (P450_3; SEQ ID NO: 7), cytochrome P450_4 (P450_4; SEQ ID NO: 8), and uracil dependent glucosyl transferase-4 (UGT_4; SEQ ID NO: 17). This tomato chassis provides a carotenoid and carotene depleted lines of the M82 tomato cultivar. Additionally, these lines are enriched with sterodial glycoalkaloids. These traits indicate a redirection of terpene precursor resources which may be utilizable by heterologous (non-native, installed) terpene or terpenoid biosynthetic genes that are engineered into these lines.

Another tomato chassis was developed to have modifications in the following endogenous terpene or terpenoid biosynthetic genes to reduce their expression (e.g. knockouts): uracil dependent glucosyl transferase-6 (UGT_6; SEQ ID NO: 19 or 41), uracil dependent glucosyl transferase-7 (UGT_7; SEQ ID NO: 20 or 42), uracil dependent glucosyl transferase-8 (UGT_8; SEQ ID NO: 21 or 43), uracil dependent glucosyl transferase-9 (UGT_9; SEQ ID NO: 22 or 44), cytochrome P450_8 (P450_8; SEQ ID NO: 12 or 34), and cytochrome P450_9 (P450_9; SEQ ID NO: 13 or 35). This tomato chassis provides a carotene and xanthophyll enriched line of the M82 tomato cultivar. These traits include an enrichment of the astaxanthin precursor, β-carotene, making it a favorable background to install a heterologous astaxanthin biosynthetic pathway.

### CRISPR/Cas Methods

In some cases, a Cas9/CRISPR system can be used to create a modification in genomic endogenous terpene or terpenoid biosynthetic gene site(s). Clustered regularly interspaced short palindromic repeats (CRISPR)/CRISPR-associated (Cas) systems are useful for, e.g. RNA-programmable genome editing (see e.g., Marraffini & Sontheimer. Nature Reviews Genetics 11:181-190 (2010); Sorek et al. Nature Reviews Microbiology 2008 6:181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12:177-186; all of which are incorporated by reference herein in their entireties). A CRISPR guide RNA can be used that can target a Cas enzyme to the desired location in the genome, where it generates a double strand break. This technique is available in the art and described, e.g. at Mali et al. Science 2013 339:823-6; which is incorporated by reference herein in its entirety and kits for the design and use of CRISPR-mediated genome editing are commercially available, e.g. the PRECISION X CAS9 SMART NUCLEASE™ System (Cat No. CAS900A-1) from System Biosciences, Mountain View, CA.

Numerous other methods for plant transformation have been developed, including biological and physical plant transformation protocols. See, for example, Rani et al., Genetic Transformation in Oilseed Brassicas—A Review INDIAN J. AGRICUL. SCI. (2013), 83 (4): 367-373 and McGinn et al., Molecular Tools Enabling Pennycress (Thlaspi Arvense) as a Model Plant and Oilseed Cash Cover Crop, PLANT BIOTECHNOL. J. (2019), 17 (4): 776-788, both of which are herein incorporated by reference in their entireties. In addition, expression vectors and in vitro culture methods for plant cell or tissue transformation and regeneration of plants are available. See, for example, Gruber & Crosby, Vectors for Plant Transformation in METHODS IN PLANT MOLECULAR BIOLOGY AND BIOTECHNOLOGY (Glick & Thompson, Ed.) (1993), 89-119, which is herein incorporated by reference in its entirety.

In general, methods to transform, modify, edit or alter plant endogenous genomic DNA include altering the plant native DNA sequence or a pre-existing transgenic sequence including regulatory elements, coding and non-coding sequences. These methods can be used, for example, to target nucleic acids to pre-engineered target recognition sequences in the genome. Such pre-engineered target sequences may be introduced by genome editing or modification. As an example, a genetically modified plant variety is generated using “custom” or engineered endonucleases such as meganucleases produced to modify plant genomes as described in WO 2009/114321, which is herein incorporated by reference in its entirety. Another site-directed engineering method is through the use of zinc finger domain recognition (e.g., artificial zinc finger nucleases), transcription activator-like (TAL) effector-DNA modifying enzyme (TALE or TALEN), site-specific modifications performed through use of a CRISPR/Cas system, expression vectors, and the like. Further discussion of such techniques is found in U.S. Pat. Nos. 10,709,151 and 11,224,237, both of which are herein incorporated by reference in their entireties.

In another example, one of skill in the art can prepare an expression cassette or expression vector that can encode the modified endogenous and/or heterologous terpene or terpenoid biosynthetic nucleic acid sequence. Plant cells can be transformed by the expression cassette or expression vector, and whole plants (and their seeds) can be generated from the plant cells that were successfully transformed with the modified endogenous and/or heterologous terpene or terpenoid biosynthetic nucleic acids. Some procedures for making such genetically modified plants and their seeds are described below.

In some embodiments, a cDNA clone encoding the heterologous terpene or terpenoid biosynthetic nucleic acid sequence is isolated from plant tissue, for example, a root, stem, leaf, seed or flower tissue. For example, cDNA clones from selected species (that encode the modified endogenous and/or heterologous terpene or terpenoid biosynthetic nucleic acid with homology to any of those described herein) are made from isolated mRNA from selected plant tissues. In another example, a nucleic acid encoding a heterologous terpene or terpenoid biosynthetic nucleic acid can be prepared by available methods or as described herein. For example, the nucleic acid encoding a modified endogenous terpene or terpenoid biosynthetic nucleic acid can be any nucleic acid with a coding region that hybridizes to a segment of a SEQ ID NOS: 1-44 nucleic acid or allelic variant thereof.

3′ Sequences: When the expression cassette is to be introduced into a plant cell, the expression cassette can also optionally include 3′ nontranslated plant regulatory DNA sequences that act as a signal to terminate transcription and allow for the polyadenylation of the resultant mRNA. The 3′ nontranslated regulatory DNA sequence preferably includes from about 300 to 1,000 nucleotide base pairs and contains plant transcriptional and translational termination sequences. For example, 3′ elements that can be used include those derived from the nopaline synthase gene of Agrobacterium tumefaciens (Bevan et ah, Nucleic Acid Research. 11:369-385 (1983)), or the terminator sequences for the T7 transcript from the octopine synthase gene of Agrobacterium tumefaciens, and/or the 3′ end of the protease inhibitor I or II genes from potato or tomato. Other 3′ elements known to those of skill in the art can also be employed. These 3′ nontranslated regulatory sequences can be obtained as described in Methods in Enzymology. 153:292 (1987). Many such 3′ nontranslated regulatory sequences are already present in plasmids available from commercial sources such as Clontech, Palo Alto, California. The 3′ nontranslated regulatory sequences can be operably linked to the 3′ terminus of the heterologous terpene or terpenoid biosynthetic nucleic acids by standard methods.

Selectable and Screenable Marker Sequences: To improve identification of transformants, a selectable or screenable marker gene can be employed with the modified endogenous terpene or terpenoid biosynthetic nucleic acids. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can ‘select’ for by chemical means, e.g., by use of a selective agent (e.g., an herbicide, antibiotic, or the like), or whether it is simply a trait that one can identify through observation or testing, i.e., by ‘screening’ (e.g., the R-locus trait). Of course, many examples of suitable marker genes are known to the art and can be employed in the practice of the invention.

Included within the terms selectable or screenable marker genes are also genes which encode a “secretable marker” whose secretion can be detected as a means of identifying or selecting for transformed cells. Examples include markers which encode a secretable antigen that can be identified by antibody interaction, or secretable enzymes that can be detected by their catalytic activity. Secretable proteins fall into a number of classes, including small, diffusible proteins detectable, e.g., by ELISA; and proteins that are inserted or trapped in the cell wall (e.g., proteins that include a leader sequence such as that found in the expression unit of extensin or tobacco PR-S).

With regard to selectable secretable markers, the use of a gene that encodes a polypeptide that becomes sequestered in the cell wall, where the polypeptide includes a unique epitope may be advantageous. Such a secreted antigen marker can employ an epitope sequence that would provide low background in plant tissue, a promoter-leader sequence that imparts efficient expression and targeting across the plasma membrane and can produce protein that is bound in the cell wall and yet is accessible to antibodies. A normally secreted wall protein modified to include a unique epitope would satisfy such requirements.

Examples of proteins suitable for modification in this manner include extensin or hydroxyproline rich glycoprotein (HPRG). For example, the maize HPRG (Stiefel et al., The Plant Cell. 2:785-793 (1990)) is well characterized in terms of molecular biology, expression, and protein structure and therefore can readily be employed. However, any one of a variety of extensins and/or glycine-rich wall proteins (Keller et ah, EMBO J. 8:1309-1314 (1989)) could be modified by the addition of an antigenic site to create a screenable marker.

Numerous other possible selectable and/or screenable marker genes will be apparent to those of skill in the art in addition to those forth herein below. Therefore, it will be understood that the discussion herein is exemplary rather than exhaustive. In light of the techniques disclosed herein and the general recombinant techniques that are known in the art, the present invention readily allows the introduction of any gene, including marker genes, into a recipient cell to generate a transformed plant cell.

Possible selectable markers for use in connection with the present invention include, but are not limited to, a neo gene (Potrykus et al., Mol. Gen. Genet. 199:183-188 (1985)) which codes for kanamycin resistance and can be selected for using kanamycin, G418, and the like; a bar gene which codes for bialaphos resistance; a gene which encodes an altered EPSP synthase protein (Hinchee et al., Bio/Technology. 6:915-922 (1988)) thus conferring glyphosate resistance; a nitrilase gene such as bxn from Klebsiella ozaenae which confers resistance to bromoxynil (Stalker et al., Science. 242:419-423 (1988)); a mutant acetolactate synthase gene (ALS) which confers resistance to imidazolinone, sulfonylurea or other ALS-inhibiting chemicals (European Patent Application 154,204 (1985)); a methotrexate-resistant DHFR gene (Thillet et al., J. Biol. Chem. 263:12500-12508 (1988)); a dalapon dehalogenase gene that confers resistance to the herbicide dalapon; or a mutated anthranilate synthase gene that confers resistance to 5-methyl tryptophan. Where a mutant EPSP synthase gene is employed, additional benefit may be realized through the incorporation of a suitable chloroplast transit peptide, CTP (European Patent Application 0 218 571 (1987)).

An illustrative embodiment of a selectable marker gene capable of being used in systems to select transformants is the gene that encode the enzyme phosphinothricin acetyltransferase, such as the bar gene from Streptomyces hygroscopicus or the pat gene from Streptomyces viridochromogenes (Ei.S. U.S. Pat. No. 5,550,318). The enzyme phosphinothricin acetyl transferase (PAT) inactivates the active ingredient in the herbicide bialaphos, phosphinothricin (PPT). PPT inhibits glutamine synthetase, (Murakami et al., Mol. Gen. Genet. 205:42-50 (1986); Twell et al., Plant Physiol. 91:1270-1274 (1989)) causing rapid accumulation of ammonia and cell death. The success in using this selective system in conjunction with monocots was surprising because of the major difficulties that have been reported in transformation of cereals (Potrykus, Trends Biotech. 7:269-273 (1989)).

Screenable markers that may be employed include, but are not limited to, a b-glucuronidase or u-id-A gene (GUS) that encodes an enzyme for which various chromogenic substrates are known; an R-locus gene, which encodes a product that regulates the production of anthocyanin pigments (red color) in plant tissues (Dellaporta et al., In: Chromosome Structure and Function: Impact of New Concepts, 18* Stadler Genetics Symposium, J. P. Gustafson and R. Appels, eds. (New York: Plenum Press) pp. 263-282 (1988)); a b-lactamase gene (Sutcliffe, Proc. Natl. Acad. Sci. USA. 75:3737-3741 (1978)), which encodes an enzyme for which various chromogenic substrates are known (e.g., PADAC, a chromogenic cephalosporin); a xylE gene (Zukowsky et al., Proc. Natl. Acad. Sci. USA. 80:1101 (1983)) which encodes a catechol dioxygenase that can convert chromogenic catechols; an a-amylase gene (Ikuta et al., Bio/technology 8:241-242 (1990)); a tyrosinase gene (Katz et al., J. Gen. Microbiol. 129:2703-2714 (1983)) which encodes an enzyme capable of oxidizing tyrosine to DOPA and dopaquinone which in turn condenses to form the easily detectable compound melanin; a b-galactosidase gene, which encodes an enzyme for which there are chromogenic substrates; a luciferase (lux) gene (Ow et al., Science. 234:856-859.1986), which allows for bioluminescence detection; or an aequorin gene (Prasher et al., Biochem. Biophys. Res. Comm. 126:1259-1268 (1985)), which may be employed in calcium-sensitive bioluminescence detection, or a green or yellow fluorescent protein gene (Niedz et al., Plant Cell Reports. 14:403 (1995)).

For example, genes from the maize R gene complex can be used as screenable markers. The R gene complex in maize encodes a protein that acts to regulate the production of anthocyanin pigments in most seed and plant tissue. Maize strains can have one, or as many as four, R alleles that combine to regulate pigmentation in a developmental and tissue specific manner. A gene from the R gene complex does not harm the transformed cells. Thus, an R gene introduced into such cells will cause the expression of a red pigment and, if stably incorporated, can be visually scored as a red sector. If a maize line carries dominant alleles for genes encoding the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 that contains the rg-Stadler allele and TR112, a K55 derivative that is r-g, b, PI. Alternatively, any genotype of maize can be utilized if the Cl and R alleles are introduced together.

The R gene regulatory regions may be employed in chimeric constructs to provide mechanisms for controlling the expression of chimeric genes. More diversity of phenotypic expression is known at the R locus than at any other locus (Coe et al., in Corn and Corn Improvement, eds. Sprague, G. F. & Dudley, J. W. (Am. Soc. Agron., Madison, WI), pp. 81-258 (1988)). It is contemplated that regulatory regions obtained from regions 5′ to the structural R gene can be useful in directing the expression of genes, e.g., insect resistance, drought resistance, herbicide tolerance or other protein coding regions. For the purposes of the present invention, it is believed that any of the various R gene family members may be successfully employed (e.g., P, S, Fc, etc.). However, one that can be used is Sn (particularly Sn: bol3). Sn is a dominant member of the R gene complex and is functionally similar to the R and B loci in that Sn controls the tissue specific deposition of anthocyanin pigments in certain seedling and plant cells, therefore, its phenotype is similar to R.

A further screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It is also envisioned that this system may be developed for population screening for bioluminescence, such as on tissue culture plates, or even for whole plant screening.

Other Optional Sequences: An expression cassette of the invention can also further comprise plasmid DNA. Plasmid vectors include additional DNA sequences that provide for easy selection, amplification, and transformation of the expression cassette in prokaryotic and eukaryotic cells, e.g., pUC-derived vectors such as pUC8, pUC9, pUC18, pUC19, pUC23, pUC119, and pUC120, pSK-derived vectors, pGEM-derived vectors, pSP-derived vectors, or pBS-derived vectors. The additional DNA sequences include origins of replication to provide for autonomous replication of the vector, additional selectable marker genes, preferably encoding antibiotic or herbicide resistance, unique multiple cloning sites providing for multiple sites to insert DNA sequences or genes encoded in the expression cassette and sequences that enhance transformation of prokaryotic and eukaryotic cells.

Another vector that is useful for expression in both plant and prokaryotic cells is the binary Ti plasmid (as disclosed in Schilperoort et ah, U.S. Pat. No. 4,940,838) as exemplified by vector pGA582. This binary Ti plasmid vector has been previously characterized by An (Methods in Enzymology. 153:292 (1987)) and is available from Dr. An. This binary Ti vector can be replicated in prokaryotic bacteria such as E. coli and Agrobacterium. The Agrobacterium plasmid vectors can be used to transfer the expression cassette to dicot plant cells, and under certain conditions to monocot cells, such as rice cells. The binary Ti vectors preferably include the nopaline T DNA right and left borders to provide for efficient plant cell transformation, a selectable marker gene, unique multiple cloning sites in the T border regions, the co/El replication of origin and a wide host range replicon. The binary Ti vectors carrying an expression cassette of the invention can be used to transform both prokaryotic and eukaryotic cells but is preferably used to transform dicot plant cells.

In Vitro Screening of Expression Cassettes: Once the expression cassette is constructed and subcloned into a suitable plasmid, it can be screened for the ability to substantially inhibit the translation of an mRNA coding for a seed storage protein by standard methods such as hybrid arrested translation. For example, for hybrid selection or arrested translation, a preselected antisense DNA sequence is subcloned into an SP6/T7 containing plasmids (as supplied by ProMega Corp.). For transformation of plants cells, suitable vectors include plasmids such as described herein. Typically, hybrid arrest translation is an in vitro assay that measures the inhibition of translation of an mRNA encoding a particular seed storage protein. This screening method can also be used to select and identify preselected antisense DNA sequences that inhibit translation of a family or subfamily of zein protein genes. As a control, the corresponding sense expression cassette is introduced into plants and the phenotype assayed.

### Transformation

DNA Delivery of the DNA Molecules into Host Cells: The present invention generally includes steps directed to introducing modifications into endogenous terpene or terpenoid biosynthetic nucleic acids and/or introducing heterologous terpene or terpenoid biosynthetic nucleic acids into a recipient cell to create a transformed cell. In some instances, the frequency of occurrence of cells taking up exogenous (foreign) DNA may be low. Moreover, it is most likely that not all recipient cells receiving DNA segments or sequences will result in a transformed cell wherein the DNA is stably integrated into the plant genome and/or expressed. Some may show only initial and transient gene expression. However, certain cells from potato species may be stably transformed, and these cells regenerated into transgenic potato plants, through the application of the techniques disclosed herein.

Transformation of the cells of the plant tissue source can be conducted by any one of a number of methods known to those of skill in the art. Examples are:

Transformation by direct DNA transfer into plant cells by electroporation (U.S. Pat. Nos. 5,384,253 and 5,472,869, Dekeyser et ah, The Plant Cell. 2:591-602 (1990)); direct DNA transfer to plant cells by PEG precipitation (Hayashimoto et al, Plant Physiol. 93:857-863 (1990)); direct DNA transfer to plant cells by microprojectile bombardment (McCabe et ah, Bio/Technology. 6:923-926 (1988); Gordon-Kamm et ah, The Plant Cell. 2:603-618 (1990); U.S. Pat. Nos. 5,489,520; 5,538,877; and 5,538,880) and DNA transfer to plant cells via infection with Agrobacterium. Methods such as microprojectile bombardment or electroporation can be carried out with “naked” DNA where the expression cassette may be simply carried on any E. coli-derived plasmid cloning vector. In the case of viral vectors, it is desirable that the system retain replication functions, but lack functions for disease induction.

One method for dicot transformation, for example, involves infection of plant cells with Agrobacterium tumefaciens using the leaf-disk protocol (Horsch et ak, Science 227:1229-1231 (1985). Monocots such as Zea mays can be transformed via microprojectile bombardment of embryogenic callus tissue or immature embryos, or by electroporation following partial enzymatic degradation of the cell wall with a pectinase-containing enzyme (U.S. Pat. Nos. 5,384,253; and 5,472,869). For example, embryogenic cell lines derived from immature Zea mays embryos can be transformed by accelerated particle treatment as described by Gordon-Kamm et al. (The Plant Cell. 2:603-618 (1990)) or U.S. Pat. Nos. 5,489,520; 5,538,877 and 5,538,880, cited above. Excised immature embryos can also be used as the target for transformation prior to tissue culture induction, selection and regeneration as described in U.S. application Ser. No. 08/112,245 and PCT publication WO 95/06128. Furthermore, methods for transformation of monocotyledonous plants utilizing Agrobacterium tumefaciens have been described by Hiei et al. (European Patent 0 604 662, 1994) and Saito et al. (European Patent 0 672 752, 1995).

Methods such as microprojectile bombardment or electroporation are carried out with “naked” DNA where the expression cassette may be simply carried on any E. coli-derived plasmid cloning vector. In the case of viral vectors, it is desirable that the system retain replication functions, but lack functions for disease induction.

The choice of plant tissue source for transformation will depend on the nature of the host plant and the transformation protocol. Useful tissue sources include callus, suspension culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells. Type I or Type II embryonic maize callus and immature embryos are preferred Zea mays tissue sources. Similar tissues can be transformed for softwood or hardwood species. Selection of tissue sources for transformation of monocots is described in detail in U.S. application Ser. No. 08/112,245 and PCT publication WO 95/06128.

The transformation is carried out under conditions directed to the plant tissue of choice. The plant cells or tissue are exposed to the DNA or RNA carrying the heterologous terpene or terpenoid biosynthetic nucleic acids for an effective period of time. This may range from a less than one second pulse of electricity for electroporation to a 2-3 days co-cultivation in the presence of plasmid-bearing Agrobacterium cells. Buffers and media used will also vary with the plant tissue source and transformation protocol. Many transformation protocols employ a feeder layer of suspended culture cells (tobacco or Black Mexican Sweet corn, for example) on the surface of solid media plates, separated by a sterile filter paper disk from the plant cells or tissues being transformed.

Electroporation: Where one wishes to introduce DNA by means of electroporation, it is contemplated that the method of Krzyzek et al. (U.S. Pat. No. 5,384,253) may be advantageous. In this method, certain cell wall-degrading enzymes, such as pectin-degrading enzymes, are employed to render the target recipient cells more susceptible to transformation by electroporation than untreated cells. Alternatively, recipient cells can be made more susceptible to transformation, by mechanical wounding.

To effect transformation by electroporation, one may employ either friable tissues such as a suspension cell cultures, or embryogenic callus, or alternatively, one may transform immature embryos or other organized tissues directly. The cell walls of the preselected cells or organs can be partially degraded by exposing them to pectin-degrading enzymes (pectinases or pectolyases) or mechanically wounding them in a controlled manner. Such cells would then be receptive to DNA uptake by electroporation, which may be carried out at this stage, and transformed cells then identified by a suitable selection or screening protocol dependent on the nature of the newly incorporated DNA.

Microprojectile Bombardment: A further advantageous method for delivering transforming DNA segments to plant cells is microprojectile bombardment. In this method, microparticles may be coated with DNA and delivered into cells by a propelling force. Exemplary particles include those comprised of tungsten, gold, platinum, and the like.

It is contemplated that in some instances DNA precipitation onto metal particles would not be necessary for DNA delivery to a recipient cell using microprojectile bombardment. In an illustrative embodiment, non-embryogenic BMS cells were bombarded with intact cells of the bacteria E. coli or Agrobacterium tumefaciens containing plasmids with either the b-glucoronidase or bar gene engineered for expression in maize. Bacteria were inactivated by ethanol dehydration prior to bombardment. A low level of transient expression of the b-glucoronidase gene was observed 24-48 hours following DNA delivery. In addition, stable transformants containing the bar gene were recovered following bombardment with either E. coli or Agrobacterium tumefaciens cells. It is contemplated that particles may contain DNA rather than be coated with DNA. Hence it is proposed that particles may increase the level of DNA delivery but are not, in and of themselves, necessary to introduce DNA into plant cells.

The microprojectile bombardment is an effective means of reproducibly stably transforming monocots that avoids the need to prepare and isolate protoplasts (Christou et al., PNAS. 84:3962-3966 (1987)), avoids the formation of partially degraded cells, and the susceptibility to Agrobacterium infection is not required. An illustrative embodiment of a method for delivering DNA into maize cells by acceleration is a Biolistics Particle Delivery System, which can be used to propel particles coated with DNA or cells through a screen, such as a stainless steel or Nytex screen, onto a filter surface covered with maize cells cultured in suspension (Gordon-Kamm et al., The Plant Cell. 2:603-618 (1990)). The screen disperses the particles so that they are not delivered to the recipient cells in large aggregates. It is believed that a screen intervening between the projectile apparatus and the cells to be bombarded reduces the size of projectile aggregate and may contribute to a higher frequency of transformation, by reducing damage inflicted on the recipient cells by an aggregated projectile.

For bombardment, cells in suspension are preferably concentrated on filters or solid culture medium. Alternatively, immature embryos or other target cells may be arranged on solid culture medium. The cells to be bombarded are positioned at an appropriate distance below the macroprojectile stopping plate. If desired, one or more screens are also positioned between the acceleration device and the cells to be bombarded. Using techniques set forth herein, one may obtain up to 1000 or more foci of cells transiently expressing a marker gene. The number of cells in a focus which express the exogenous gene product 48 hours post-bombardment often range from about 1 to 10 and average about 1 to 3.

In bombardment transformation, one may optimize the prebombardment culturing conditions and the bombardment parameters to yield the maximum numbers of stable transformants. Both the physical and biological parameters for bombardment can influence transformation frequency. Physical factors are those that involve manipulating the DNA/microprojectile precipitate or those that affect the path and velocity of either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmid DNA.

One may wish to adjust various bombardment parameters in small scale studies to fully optimize the conditions and/or to adjust physical parameters such as gap distance, flight distance, tissue distance, and helium pressure. One may also minimize the trauma reduction factors (TRFs) by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. Execution of such routine adjustments will be known to those of skill in the art.

### Plant Propagation

An Example of Production and Characterization of Stable Transgenic Plants: After effecting delivery of a heterologous terpene or terpenoid biosynthetic nucleic acid to recipient cells by any of the methods discussed above, the transformed cells can be identified for further culturing and plant regeneration. As mentioned above, to improve the ability to identify transformants, one may desire to employ a selectable or screenable marker gene as, or in addition to, the modified endogenous and/or heterologous terpene or terpenoid biosynthetic gene nucleic acids. In this case, one would then generally assay the potentially transformed cell population by exposing the cells to a selective agent or agents, or one would screen the cells for the desired marker gene trait.

Selection: An exemplary embodiment of methods for identifying transformed cells involves exposing the bombarded cultures to a selective agent, such as a metabolic inhibitor, an antibiotic, herbicide or the like. Cells which have been transformed and have stably integrated a marker gene conferring resistance to the selective agent used, will grow and divide in culture. Sensitive cells will not be amenable to further culturing.

To use the har-bialaphos or the EPSPS-glyphosate selective system, bombarded tissue is cultured for about 0-28 days on nonselective medium and subsequently transferred to medium containing from about 1-3 mg/l bialaphos or about 1-3 mM glyphosate, as appropriate. While ranges of about 1-3 mg/l bialaphos or about 1-3 mM glyphosate can be employed, it is proposed that ranges of at least about 0.1-50 mg/l bialaphos or at least about 0.1-50 mM glyphosate will find utility in the practice of the invention. Tissue can be placed on any porous, inert, solid or semi solid support for bombardment, including but not limited to filters and solid culture medium. Bialaphos and glyphosate are provided as examples of agents suitable for selection of transformants, but the technique of this invention is not limited to them.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. The R-locus is useful for selection of transformants from bombarded immature embryos. In a similar fashion, the introduction of the Cl and B genes will result in pigmented cells and/or tissues.

The enzyme luciferase is also useful as a screenable marker in the context of the present invention. In the presence of the substrate luciferin, cells expressing luciferase emit light which can be detected on photographic or X-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time.

It is further contemplated that combinations of screenable and selectable markers may be useful for identification of transformed cells. For example, selection with a growth inhibiting compound, such as bialaphos or glyphosate at concentrations below those providing 100% inhibition followed by screening of growing tissue for expression of a screenable marker gene such as luciferase would allow one to recover transformants from cell or tissue types that are not amenable to selection alone. In an illustrative embodiment embryogenic Type II callus of Zea mays L. can be selected with sub-lethal levels of bialaphos. Slowly growing tissue was subsequently screened for expression of the luciferase gene and transformants can be identified.

Regeneration and Seed Production: Cells that survive the exposure to the selective agent, or cells that have been scored positive in a screening assay, are cultured in media that supports regeneration of plants. One example of a growth regulator that can be used for such purposes is dicamba or 2,4-D. However, other growth regulators may be employed, including NAA, NAA+2,4-D or perhaps even picloram. Media improvement in these and like ways can facilitate the growth of cells at specific developmental stages. Tissue can be maintained on a basic media with growth regulators until sufficient tissue is available to begin plant regeneration efforts, or following repeated rounds of manual selection, until the morphology of the tissue is suitable for regeneration, at least two weeks, then transferred to media conducive to maturation of embryoids. Cultures are typically transferred every two weeks on this medium. Shoot development signals the time to transfer to medium lacking growth regulators.

The transformed cells, identified by selection or screening and cultured in an appropriate medium that supports regeneration, can then be allowed to mature into plants. Developing plantlets are transferred to soilless plant growth mix, and hardened, e.g., in an environmentally controlled chamber at about 85% relative humidity, about 600 ppm CO2, and at about 25-250 microeinsteins/sec-mª of light. Plants can be matured either in a growth chamber or greenhouse. Plants are regenerated from about 6 weeks to 10 months after a transformant is identified, depending on the initial tissue. During regeneration, cells are grown on solid media in tissue culture vessels. Illustrative embodiments of such vessels are petri dishes and Plant Con™. Regenerating plants can be grown at about 19° C. to 28° C. After the regenerating plants have reached the stage of shoot and root development, they may be transferred to a greenhouse for further growth and testing.

Mature plants are then obtained from cell lines that are known to express the trait. In some embodiments, the regenerated plants are self-pollinated. In addition, pollen obtained from the regenerated plants can be crossed to seed grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture are of interest if the traits are to be commercially useful.

Regenerated plants can be repeatedly crossed to inbred plants to introgress the modified endogenous and/or heterologous terpene or terpenoid biosynthetic nucleic acids into the genome of the inbred plants. This process is referred to as backcross conversion. When a sufficient number of crosses to the recurrent inbred parent have been completed to produce a product of the backcross conversion process that is substantially isogenic with the recurrent inbred parent except for the presence of the introduced modifications to endogenous terpene or terpenoid biosynthetic nucleic acids and/or heterologous terpene or terpenoid biosynthetic nucleic acids, the plant is self-pollinated at least once to produce a homozygous backcross converted inbred containing the modified endogenous and/or heterologous terpene or terpenoid biosynthetic nucleic acids. Progeny of these plants are true breeding.

Alternatively, seed from transformed monocot plants regenerated from transformed tissue cultures is grown in the field and self-pollinated to generate true breeding plants.

Seed from the fertile transgenic plants can then be evaluated for the presence and/or expression of the modified endogenous and/or heterologous terpene or terpenoid biosynthetic nucleic acids. Transgenic plant and/or seed tissue can be analyzed for a reduction in transcription of the modified endogenous and/or heterologous terpene or terpenoid biosynthetic nucleic acids using standard methods such as SDS polyacrylamide gel electrophoresis, liquid chromatography (e.g., HPLC), gas chromatography or other means of detecting a product of terpene or terpenoid biosynthetic activity (e.g., increased production of terpenes or terpenoids).

Once a transgenic seed containing the modified endogenous and/or heterologous terpene or terpenoid biosynthetic nucleic acid sequence and having a reduced expression of the modified endogenous and/or heterologous terpene or terpenoid biosynthetic protein is identified, the seed can be used to develop true breeding plants. The true breeding plants are used to develop a line of plants with the modified endogenous and/or heterologous terpene or terpenoid biosynthetic nucleic acids while still maintaining other desirable functional agronomic traits. Adding the modified endogenous and/or heterologous terpene or terpenoid biosynthetic nucleic acid along with normal growth of the plant can be accomplished by back-crossing with this trait and with plants that do not exhibit this trait and studying the pattern of inheritance in segregating generations. Those plants expressing the target trait in a dominant fashion are preferably selected.

Back-crossing is carried out by crossing the original fertile transgenic plants with a plant from an inbred line exhibiting desirable functional agronomic characteristics while not necessarily expressing the trait of reduced expression of endogenous terpene or terpenoid biosynthetic genes. The resulting progeny are then crossed back to the parent that expresses the increased reduced CIS trait. The progeny from this cross will also segregate so that some of the progeny carry the trait and some do not. This back-crossing is repeated until an inbred line with the desirable functional agronomic traits, and with expression of the trait involving reduced CIS. Such expression of reduced CIS can be expressed in a dominant fashion.

Determination of Stably Transformed Plant Tissues: To confirm the presence of the modified endogenous and/or heterologous terpene or terpenoid biosynthetic nucleic acids in the regenerating plants, or seeds or progeny derived from the regenerated plant, a variety of assays may be performed. Such assays include, for example, molecular biological assays available to those of skill in the art, such as Southern and Northern blotting and PCR; biochemical assays, such as detecting the presence of a protein product, e.g., by immunological means (ELISAs and Western blots) or by enzymatic function; plant part assays, such as leaf, seed or root assays; and also, by analyzing the phenotype of the whole regenerated plant.

Whereas DNA analysis techniques may be conducted using DNA isolated from any part of a plant, RNA may only be expressed in particular cells or tissue types and so RNA for analysis can be obtained from those tissues. PCR techniques may also be used for detection and quantification of RNA produced from introduced modifications to endogenous terpene or terpenoid biosynthetic nucleic acids and/or heterologous terpene or terpenoid biosynthetic nucleic acids. PCR also be used to reverse transcribe RNA into DNA, using enzymes such as reverse transcriptase, and then this DNA can be amplified by use of conventional PCR techniques. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and also demonstrate the presence or absence of an RNA species.

While Southern blotting and PCR may be used to detect the modified endogenous and/or heterologous terpene or terpenoid biosynthetic nucleic acid in question, they do not provide information as to whether the preselected DNA segment is being expressed. Expression may be evaluated by specifically identifying the protein products of the introduced heterologous terpene or terpenoid biosynthetic nucleic acids or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange, liquid chromatography or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques.

The expression of a gene product can also be determined by evaluating the phenotypic results of its expression. These assays also may take many forms including but not limited to analyzing changes in the chemical composition, morphology, or physiological properties of the plant. Chemical composition may be altered by expression of preselected DNA segments encoding storage proteins which change amino acid composition and may be detected by amino acid analysis.

The following non-limiting Examples illustrate how aspects of the technology have been developed and can be made and used. Additional embodiments of the disclosure reside in specific examples and data described in more detail herein.

### EXAMPLES

### Example 1: Materials and Methods

This example describes some of the materials and methods used in developing the technology.

### 1. Tomato Genome Resources.

The tomato reference genome generated from the processing variety Heinz 1706 has been updated using a long-read de novo assembly approach the current version, SL4.0 (782 Mb), encodes 34,075 genes. High quality long-read genome assemblies and replicated gene expression profiling datasets that span major developmental stages of vegetative and reproductive tomato tissues including fruit development are now available for M82, a processing tomato used extensively in tomato genetic and biotechnology research.

2. M82 dsRed/SpCas9 Tomato Reporter Line.

An M82 tomato line was developed that expresses SpCas9 and dsRed to enable rapid detection of gene-editing in Fast-TrACC-derived tomato seedlings via dsRed-negative plantlets. dsRed and Cas9 are driven respectively by the AtUbi10 and 35S promoter for strong, constitutive expression.

3. Agrobacterium tumefaciens-Mediated Transient Expression in Tomato Fruit.

Adapted from Orzaez, et. al., Plant Physiology (2006). This protocol provides a method for transient metabolism of isoprenoid pathways and a β-glucuronidase reporter system within M82 tomato fruit.

### A. Four/Five Days Before Infiltration:

Transform fresh agrobacterium with constructs and plate onto LB agar with proper antibiotics.
(i) Rif+Gent+Binary Vector selection for GV3101.
(ii) Rif+Binary Vector selection for LBA4404.

### B. Two/Three Days Before Infiltration

1. Colony PCR confirm 3 or more colonies per construct.
2. Make primary cultures in 5 mL LB with necessary antibiotics from confirmed colonies while aiming to use at least 3 or more positive colonies per culture to minimize confounding mutations.
3. Culture at 28-30° C. with 200 RPM for two days from fresh transformant or one day from glycerol stock.

### C. One Day Before Infiltration

1. Make secondary cultures in 25 mL LB or Orzaez culturing buffer with necessary antibiotics. Generally, 500 to 1000 μL of concentrated primary will give OD600. 6-1 after 14-15 hours. Culture O/N at 28-30° C. with 200 RPM.

### D. Day of Infiltration

1. Ensure OD600 is between 1.0-2.5. Back dilute if necessary, ensuring cells can double twice.
2. Centrifuge cultures at 4,000 RPM for 10 minutes, resuspend in 25 mL of sterile water, and centrifuge at 4,000 RPM for 10 minutes again to water wash the pellet.
3. Complete the water wash a second time.
4. Centrifuge cultures at 4,000 RPM for 10 minutes and resuspend in 25 mL of induction media.
   i. Andersen's induction media is sterile water with 40 mg/L acetosyringone.
   ii. Induction media from Orzaez et al., 2006 is an option as well.
   iii. Reist's infiltration media is adapted from the above two and is most commonly used:
      a. For 250 mL infiltration media: 0.508 g MgCl₂·6H₂O; 0.488 g MES; Buffer to pH of 5.6 (I use a 1.25 M NaOH stock); 10 mg acetosyringone dissolved in 1 mL DMSO
5. Incubate at 28-30° C. with 200 RPM for at least 2 hours.
6. After 2 hours, combine combinatorial strains together in 1:1:1 . . . fashion.
7. Infiltrate into pericarp of a detached fruit using a clean syringe and a 0.22-gauge needle.
a. Can do multiple injection sites around the fruit. This is recommended.
b. Ensure cell suspension is well mixed before each injection.
c. Track the infiltration progress as best as possible. This is usually easier with fruit between mature green and pink developmental stages.
8. Infiltrate till entire surface is infiltrated.
9. Optional: mark areas well infiltrated with a sharpie.
   a. Incubate fruit in suitable place-ideally warm, 21-25° C., and intermediately lit by growth lights or ambient room light. Incubating the fruit at 29° C. or greater is detrimental to the success of transformation.
10. After 7-10 days, take fruit samples, paying attention to the areas which infiltrated well.
11. For triterpenes and heavier/minimally volatile terpenes, lyophilize tissue after flash freezing in liquid nitrogen and store dehydrated at −80° C. For diterpenes and lighter/volatile, do not lyophilize. Instead, just flash freeze and grind with a mortar and pestle to form a homogenous tissue for organic solvent sampling and/or storage at −80° C.

| Orzaez Culturing Media | Orzaez Induction/Infiltration Media |
|---|---|
| For 400 mL: | For 250 mL: |
| 2 g beef Extract | 0.238 g MgCl₂ or 0.508 g MgCl₂•6H₂O |
| 0.4 g yeast extract | 0.488 g MES |
| 2 g peptone | 312.5 uL of 160 mM acetosyringone stock |
| 2 g sucrose | Buffer to pH of 5.6 (I still use NaOH stock) |
| 0.09625 g MgSO₄ or 0.197 g | Sterilized water to 250 mL |
| MgSO₄•7H₂O | |
| 0.7808 g MES | |
| Buffer to pH of 5.6 (I use a NaOH stock to | |
| do so) | |
| 50 uL of 160 mM acetosyringone stock | |
| (For 160 mM stock: 0.0314 g | |
| acetosyringone into 1 mL DMSO. Make | |
| fresh each experiment.) | |
| Sterilized water to 400 mL | |
| Add necessary antibiotics | |

### 4. Fast-TrACC Fast-Treated Agrobacterium Co-Culture

To deliver gene editing reagents that avoid or minimize the use of tissue culture, editing methods focus on the meristem—the stem cell niche located in growing apices that is the progenitor to all above-ground organs, including leaves and flowers. In one approach, differentiated somatic cells are edited and induced to form meristems by delivering developmental regulators, which include transcription factors such as WUSHEL and SHOOT MERISTEMLESS, whose ectopic expression induces meristem formation. In some cases, we also co-deliver the cytokinin biosynthesis gene, isopentenyl transferase (ipt), since hormones are also important in meristem identity and function. The resulting edited shoots ultimately produce flowers and seed that transmit the gene edits to the next generation.

To determine the best combination of developmental regulators and ipt to induce gene-edited shoots, a method called Fast-TrACC (Fast-Treated Agrobacterium Co-Culture) was developed wherein developmental regulators and hormone biosynthesis genes are delivered by Agrobacterium in various combinations to seedlings germinated in liquid medium32 (FIG. 11, Panel A). Typically about 20 days after delivery, growths begin to emerge on the seedlings, some of which develop into shoots. These shoots can either be transgenic, gene edited or both. If the shoots are transferred to solid medium, they develop roots and ultimately flowers and edited seed. An alternative to Fast-TrACC involves adding the developmental regulators to soil-grown plants that have had existing shoots removed by pruning (FIG. 11, Panel B). New transgenic or gene-edited shoots emerge from the treated sites. Using Fast-TrACC, one gene edited shoot can be generated, on average, from every six treated tomato seedlings. From soil-grown plants, typically one gene edited shoot is obtained for every one to two treated plants. Fast-TrACC methods can be used for high throughput mutagenesis because large numbers of gene edited shoots can be easily generated in growth chambers and/or incubators.

### Example 2: Determining Tomato Fruit Tri- and Tetaterpene Associated P450s and UGTs

To identify P450s and UGTs involved in tri- and tetra-terpene biosynthesis in tomato fruit, expression abundances of 42 replicated mRNA-seq datasets from an array of M82 vegetative and reproductive tissues from the NCBI Sequence Read Archive were determined and used to construct gene co-expression clusters using Clust126. Using a combination of (i) knowledge of TPS, P450s, and UGTs as described above, (ii) expression profiles in fruit vs vegetative tissue, (iii) gene coexpression, and (iv) physical clustering in the genome, 3 terpene synthases (TPS), 11 cytochrome P450s, and 36 Uracil dependent glucosyl transferase-4 (UGTs) were identified as putatively associated with production of tri- and tetra-terpenes in tomato fruit.

### A. Generation of Gene Edited TPS, P450 and UGT Knock-Out Lines.

An efficient method for generating gene edited tomato shoots is to treat M82 seedlings with a mixture of two Agrobacterium strains, one expressing the developmental regulators, nos:ZmWUS2 and 35S:ipt, and the other expressing gene editing reagents. Once shoots emerge, dsRed-negative shoots are identified using a NIGHTSEA dual fluorescent protein flashlight and transferred to root-inducing medium for 1-2 weeks and then to soil. With an approximate success rate of one edited plant per six seedlings using the Fast-TrACC method, 18 tomato seedlings are screened with all TPSs and candidate P450s and UGTs to ensure generation of at least three gene editing events per target.

### B. Screening and Confirmation of Putative Function of Knock-Out Lines.

While the Fast-TrACC method has a high success rate of gene editing^{32,35}, we need to confirm the gene editing event at the molecular level prior to metabolite profiling.

### (i). Confirmation of Gene Editing Event.

Optimally, our sgRNA design will generate a deletion of >100 bp, such that we can readily screen for knock-outs in dsRed-negative plants for mono- and bi-allelic deletion events via PCR and agarose gels. We will use the Thermo Scientific Phire Plant Direct PCR Master Mix, which couples DNA isolation with PCR to rapidly screen for gene editing activity. If no deletions are detected, DNA from the dsRed-negative plant will be cloned and Sanger sequenced to confirm gene editing activity in the form of small deletions/insertions or substitutions.

### (ii). Confirmed Gene Knock-Outs Will be Advanced for Terpenoid Profiling.

Single gene edited lines will be analyzed for terpenoid profiles using targeted gas chromatography (GC)-MS and liquid chromatography (LC-MS) with a focus on removal of known metabolites and reduced biochemical complexity in comparison to wild type M82. Procedures are established as previously described and suitable for high-throughput analysis. As we will analyze a minimum of three independent gene editing events per target, we will have built in replication. In brief, we will harvest 80-100 mg of mature fruit and hexane extraction of the non-polar fraction and 80% methanol for the polar fraction will be subjected to GC- and LC-MS, respectively. Transcripts of all TPS targets have been detected in these tissues and we expect that their inactivation yields a predictable chemotype with the function of the TPSs established. We will follow the same procedure for the candidate P450s and UGTs where we will use a metabolomics procedure developed and targeted for terpenoids.

### Example 3: Terepene/Terpenoid Pathway Overexpression Transgenic Tomato Plants

Transgenic tomato plant inventory which express key isoprenoid pathway target genes using fruit specific promoters and terminators were made by genetically engineering the tomato genome using Agrobacterium-mediated transformation. Tomato fruit specific promoters include a PG promoter (Ruan, X-M, et. al., Horticulture Research (2024); Atkinson, et. al., Plant Molecular Biology (1998), both of which are incorporated herein by reference in their entirety).

TABLE 6
Pathway Overexpression Transgenic Plants
Column 1 & 2

| Transgenic | Heterologous |
|---|---|
| Plant | Genes |
| ID | Present |
| 828-14 | DgTPS1, |
| | CfDXS, |
| | CfGGPPS |
| 829-1 | DgTPS1 |
| 829-7 | DgTPS1 |
| 829-12 | DgTPS1 |
| 829-13 | DgTPS1 |
| 829-16 | DgTPS1 |
| 829-17 | DgTPS1 |
| 831-2 | DgTPS1, |
| | EICYP726A27 |
| 831-4 | DgTPS1, |
| | EICYP726A27 |
| 831-5 | DgTPS1 |
| | EICYP726A27 |
| 831-12 | DgTPS1, |
| | EICYP726A27 |
| 831-13 | DgTPS1, |
| | EICYP726A27 |
| 831-15 | DgTPS1 |
| | EICYP726A27 |
| 831-17 | DgTPS1, |
| | EICYP726A27 |
| 831-20 | DgTPS1, |
| | EICYP726A27 |
| 831-21 | DgTPS1, |
| | EICYP726A27 |
| 831-23 | DgTPS1 |
| | EICYP726A27 |
| 831-24 | DgTPS1, |
| | EICYP726A27 |
| 831-25 | DgTPS1, |
| | EICYP726A27 |
| p833-1-1 | TP-SbTPS10, |
| | TP-AtFPPS |
| p833-1-2 | TP-SbTPS10, |
| | TP-AtFPPS |
| 833-2 | TP-SbTPS10, |
| | TP-AtFPPS |
| 833-5 | TP-SbTPS10, |
| | TP-AtFPPS |
| 833-8 | TP-SbTPS10, |
| | TP-AtFPPS |
| 833-9 | TP-SbTPS10, |
| | TP-AtFPPS |
| 833-12 | TP-SbTPS10, |
| | TP-AtFPPS |
| 833-13 | TP-SbTPS10, |
| | TP-AtFPPS |
| 833-14 | TP-SbTPS10, |
| | TP-AtFPPS |
| 833-15 | TP-SbTPS10, |
| | TP-AtFPPS |
| 833-20 | TP-SbTPS10, |
| | TP-AtFPPS |
| 833-23 | TP-SbTPS10, |
| | TP-AtFPPS |
| 834-1 | PvHVS, |
| | CfDXS, |
| | CfGGPPS |
| 834-2 | PvHVS, |
| | CfDXS, |
| | CfGGPPS |
| 834-3 | PvHVS, |
| | CfDXS, |
| | CfGGPPS |
| 834-4 | PvHVS, |
| | CfDXS, |
| | CfGGPPS |
| 834-5 | PvHVS, |
| | CfDXS, |
| | CfGGPPS |
| 834-6 | PvHVS, |
| | CfDXS, |
| | CfGGPPS |
| 834-7 | PvHVS, |
| | CfDXS, |
| | CfGGPPS |
| 834-8 | PvHVS, |
| | CfDXS, |
| | CfGGPPS |
| 834-10 | PvHVS, |
| | CfDXS, |
| | CfGGPPS |
| 834-11 | PvHVS, |
| | CfDXS, |
| | CfGGPPS |
| 834-12 | PvHVS, |
| | CfDXS, |
| | CfGGPPS |
| 834-13 | PvHVS, |
| | CfDXS, |
| | CfGGPPS |
| 834-14 | PvHVS, |
| | CfDXS, |
| | CfGGPPS |
| 834-16 | PvHVS |
| | CfDXS, |
| | CfGGPPS |
| 834-18 | PvHVS, |
| | CfDXS, |
| | CfGGPPS |
| 835-3 | PvHVS |
| 835-4 | PvHVS |
| 835-5 | PvHVS |
| 835-6 | PvHVS |
| 835-7 | PvHVS |
| 843-1 | TP-SbTPS10, |
| | TP-AtFPPS |
| | CfDXS |
| 843-2 | TP-SbTPS10, |
| | TP-AtFPPS, |
| | CfDXS |
| 843-3 | TP-SbTPS10, |
| | TP-AtFPPS |
| | CfDXS |
| 843-6 | TP-SbTPS10, |
| | TP-AtFPPS |
| | CfDXS |
| 843-7 | TP-SbTPS10, |
| | TP-AtFPPS, |
| | CfDXS |
| 843-8 | TP-SbTPS10, |
| | TP-AtFPPS, |
| | CfDXS |
| 843-9 | TP-SbTPS10, |
| | TP-AtFPPS, |
| | CfDXS |
| 843-10 | TP-SbTPS10, |
| | TP-AtFPPS, |
| | CfDXS |
| 843-14 | TP-SbTPS10, |
| | TP-AtFPPS, |
| | CfDXS |
| 843-16 | TP-SbTPS10, |
| | TP-AtFPPS, |
| | CfDXS |
| 843-17 | TP-SbTPS10, |
| | TP-AtFPPS, |
| | CfDXS |
| 845-1 | CfTPS2, |
| | OmTPS3 |
| 845-4 | CfTPS2, |
| | OmTPS3 |
| 845-5 | CfTPS2, |
| | OmTPS3 |
| 845-6 | CfTPS2, |
| | OmTPS3 |
| 845-7 | CfTPS2, |
| | OmTPS3 |
| 845-8 | CfTPS2, |
| | OmTPS3 |
| 845-9 | CfTPS2, |
| | OmTPS3 |
| 845-10 | CfTPS2, |
| | OmTPS3 |
| 845-11 | CfTPS2, |
| | OmTPS3 |
| 845-12 | CfTPS2, |
| | OmTPS3 |
| 845-13 | CfTPS2, |
| | OmTPS3 |
| 845-14 | CfTPS2, |
| | OmTPS3 |
| 845-17 | CfTPS2, |
| | OmTPS3 |
| 845-18 | CfTPS2, |
| | OmTPS3 |
| 845-19 | CfTPS2, |
| | OmTPS3 |
| 845-23 | CfTPS2, |
| | OmTPS3 |
| 845-24 | CfTPS2, |
| | OmTPS3 |
| 849-9 | CfDXS, |
| | CfGPPS |
| 849-17 | CfDXS, |
| | CfGPPS |
| 849-18 | CfDXS, |
| | CfGPPS |
| 849-21 | CfDXS, |
| | CfGPPS |

### Example 4: Sequencing of Select T1 Generation Tomato Plants with Modifications to Terepene or Terpenoid Biosynthetic Genes

Libraries were made from each of these individuals and sequenced across four flow cells. The resulting sequences were pooled for analysis. Flye assemblies of each library were compared to the genome reference to check for major structural alterations. The modified terepene or terpenoid biosynthetic genes are shown for each subject tomato plant tested with confirmation of the modifications. Summary of the sequencing is as follows:

TABLE 7A
Sequence verification of modified tomato plants

| Subject | 1 |
|---|---|
| Genome | No sign of structural rearrangements |
| Vector | A partial copy of the vector including the gRNA region, SlUbi promoter, and |
| | nptii, but lacking CAS9, the Amcyan CDS and any backbone sequence is present |
| | at Chr7: 55,347,230-55,348,040. Homozygous (15 read coverage). |
| P450_5 | Completely wt with 16 reads covering exon 1 |
| P450_6 | 8/8 reads support a 1 bp insertion |
| P450_7 | 11 bp deletion in all 10 reads |
| UGT_5 | 2 bp deletion in all 9 reads |
| Subject | 2 |
| Genome | Inversions detected on chr 3 (P450_1/P450_2) and chr12 (unique to 721-7-2) |
| Vector | There is a mostly complete LB-RB insertion at Chr6: 43329202-43,330,372 |
| | presumably disrupting function of SollycM82_v1.6G039560 which encodes a |
| | putative “Protease-associated RING/U-Box zinc finger family protein.” This |
| | insertion is missing the end of the LUC CDS (343 bp) and terminator. All |
| | gRNAs are present in the 3 reads covering that region of the plasmid. |
| CrtRB2 | 2/5 reads have a 4 bp deletion. The remaining 3 reads have a 1 bp insertion |
| PSY1 | All reads (5) have a ~100 bp chunk of altered sequence |
| P450_1 | All reads (~6) support an inversion between P450_1 and P450_2 |
| P450_2 | All reads (~6) support an inversion between P450_1 and P450_2 |
| UGT_4 | All reads (10) support a 72 bp deletion, 1 and 2 bp deletions, a 1 bp |
| | insertion, and several SNPs. |
| P450_3 | All reads (10) support a 152 bp deletion |
| P450_4 | All reads (14) have two 1 bp insertions. |

| | |
|---|---|
| Subject | 3 |
| Genome | Inversion detected on chr3 (P450_1/P450_2) |
| Vector | Several reads support the Chr6 insertion identified in 721-7-2. No additional |
| | insertions detected |
| CrtRB2 | 7 of 11 reads have a 4 bp deletion. 3 of the remaining reads have the 1 bp |
| | insertion. The remaining read has messy sequence across this region. |
| PSY1 | All reads (5) have a ~100 bp chunk of altered sequence |
| P450_1 | All reads (~8) support an inversion between P450_1 and P450_2 |
| P450_2 | All reads (~8) support an inversion between P450_1 and P450_2 |
| UGT_4 | All reads (9) support a 72 bp deletion, 1 and 2 bp deletions, a 1 bp |
| | insertion, and several SNPs. |
| P450_3 | All reads (3) support a 152 bp deletion |
| P450_4 | All reads (6) support a 62 bp deletion |

| Subject | 4 |
|---|---|
| Genome | Inversion detected on chr3 (P450_1/P450_2) |
| Vector | No vector positive reads identified |
| CrtRB2 | 2 of 5 reads have a 4 bp deletion while the remaining 3 have a |
| | 7 bp deletion at the same position |
| PSY1 | All reads (7) have a ~100 bp chunk of altered sequence |
| P450_1 | All reads (~3) support an inversion between P450_1 and P450_2 |
| P450_2 | All reads (~3) support an inversion between P450_1 and P450_2 |
| UGT_4 | All reads (4) support a 72 bp deletion, 1 and 2 bp deletions, a 1 bp |
| | insertion, and several SNPs. |
| P450_3 | All reads (7) support a 152 bp deletion |
| P450_4 | All reads (4) support a 62 bp deletion |
| Subject | 5 |
| Genome | No sign of structural rearrangements |
| Vector | No vector positive reads identified |
| UGT_6 | There are two gene models (identical over 20 kb) representing |
| | this gene in this annotation. 13 uniquely mapping reads support |
| | a 4 bp deletion in SollycM82_v1.9G023420. 7 uniquely mapping |
| | reads support a 4 bp deletion in SollycM82_v1.9G023470. |
| UGT_7 | 6/6 reads have a 1 bp insertion |
| P450_8 | 4/4 reads support a 35 bp insertion |
| UGT_8 | 8/8 reads support a 4 bp deletion |
| UGT_9 | 6/6 reads support both a 2 bp and a 5 bp deletion |
| P450_9 | 6/6 reads support a 474 bp deletion |

| | |
|---|---|
| Subject | 6 |
| Genome | No sign of structural rearrangements |
| Vector | No vector positive reads identified |
| UGT_6 | There are two gene models (identical over 20 kb) representing this gene |
| | in this annotation. 4 uniquely mapping reads support a 4 bp deletion in |
| | SollycM82_v1.9G023420.1 while 3 reads are wt. 3 uniquely mapping reads |
| | support a 4 bp deletion in SollycM82_v1.9G023470 while 4 reads are wt. |
| UGT_7 | 5/6 reads have a 1 bp insertion and the other read has multiple SNPs |
| | within a few bp suggesting low sequence quality for that read |
| P450_8 | 10/10 reads support a 35 bp insertion |
| UGT_8 | 11/11 reads support a 4 bp deletion |
| UGT_9 | 10/10 reads support the 2 bp deletion 8/10 support the 5 bp deletion |
| | while the remaining two have messy sequence across that region with |
| | other smaller deletions (likely poor quality reads) |
| P450_9 | 4/4 reads support a 474 bp deletion |

TABLE 8
Library information

| | | | Median | Mean | read_length | | | |
|---|---|---|---|---|---|---|---|---|
| Subject | # reads | # bases | read_length | read_length | stdev | n50 | mean_qual | median_qual |
| 1 | 53,799 | 618,698,847 | 3,687 | 11,500 | 15,787 | 30,371 | 16 | 19 |
| | 40,273 | 440,133,089 | 4,037 | 10,929 | 14,737 | 26,901 | 15 | 17 |
| | 106,535 | 924,531,649 | 3,219 | 8,678 | 13,851 | 26,758 | 17 | 19 |
| | 1,054,839 | 7,832,761,440 | 1,204 | 7,426 | 13,961 | 29,755 | 18 | 21 |
| 2 | 58,140 | 558,984,226 | 3,559 | 9,615 | 13,615 | 25,520 | 17 | 19 |
| | 44,598 | 418,249,613 | 3,725 | 9,378 | 12,844 | 22,778 | 15 | 17 |
| | 135,850 | 1,059,160,000 | 3,247 | 7,797 | 12,244 | 22,265 | 16 | 19 |
| | 773,885 | 4,981,314,098 | 1,292 | 6,437 | 12,295 | 25,079 | 18 | 21 |
| 3 | 59,425 | 682,137,864 | 4,650 | 11,479 | 14,165 | 27,319 | 17 | 19 |
| | 45,484 | 496,779,988 | 4,897 | 10,922 | 13,423 | 24,421 | 16 | 17 |
| | 121,542 | 1,119,998,990 | 3,552 | 9,215 | 12,932 | 24,470 | 16 | 19 |
| | 413,861 | 4,461,197,927 | 3,552 | 10,780 | 14,733 | 28,889 | 16 | 19 |
| 4 | 53,691 | 547,550,399 | 3,547 | 10,198 | 15,312 | 30,109 | 16 | 18 |
| | 40,390 | 390,678,757 | 3,554 | 9,673 | 14,379 | 26,574 | 15 | 17 |
| | 109,024 | 829,445,413 | 2,802 | 7,608 | 13,021 | 25,058 | 16 | 19 |
| | 163,855 | 1,182,636,105 | 1,282 | 7,218 | 13,968 | 31,168 | 16 | 18 |
| 5 | 54,752 | 626,550,333 | 5,260 | 11,443 | 14,476 | 26,228 | 16 | 18 |
| | 42,389 | 458,718,243 | 5,416 | 10,822 | 13,555 | 23,358 | 15 | 17 |
| | 90,008 | 815,808,075 | 3,609 | 9,064 | 12,951 | 22,422 | 16 | 19 |
| | 383,779 | 4,045,760,000 | 3,632 | 10,542 | 15,202 | 28,551 | 15 | 17 |
| 6 | 46,347 | 558,395,427 | 4,356 | 12,048 | 15,390 | 29,563 | 16 | 19 |
| | 34,736 | 396,404,300 | 4,415 | 11,412 | 14,517 | 27,027 | 15 | 17 |
| | 83,246 | 788,524,033 | 3,549 | 9,472 | 13,916 | 26,272 | 16 | 19 |
| | 347,922 | 4,117,178,431 | 3,554 | 11,834 | 16,362 | 32,063 | 15 | 17 |

TABLE 9
Flye assembly statistics

| Subject | Assembly length | N50 | # Contigs | Mean coverage |
|---|---|---|---|---|
| 1 | 791,047,663 | 4,343,563 | 884 | 11 |
| 2 | 781,364,567 | 1,061,489 | 1,946 | 8 |
| 3 | 786,299,345 | 1,172,751 | 1,784 | 7 |
| 4 | 509,086,941 | 163,369 | 5,367 | 4 |
| 5 | 777,302,028 | 693,108 | 2,610 | 7 |
| 6 | 780,244,369 | 827,282 | 1,974 | 6 |

### Example 5: Tomato Fruit Expression of Heterologous Terpene or Terpenoid Biosynthetic Gene

The following constructs were made for expression in tomato fruit.

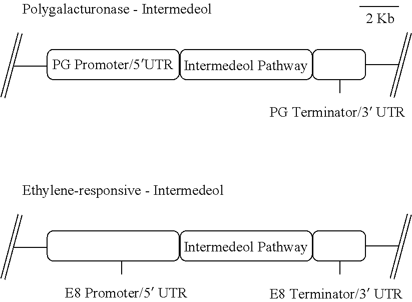

TABLE 10
Screening results for transgenic tomato expressing heterologous
terpene or terpenoid biosynthetic genes.

| Metabolic | Number of | Number of | | Range of | | |
|---|---|---|---|---|---|---|
| Engineering | Phenotyped | Phenotyped | Ratio of | Metabolic | | |
| Design (MED) | Transgenic | Transgenic | Screened | Yield of | | Number |
| of Stable | Events | Events | Events | Positive | | of Fruit |
| Transgenic | Positive for | Negative for | Positive for | Heterologous | | Sampled |
| Plant | Heterologous | Heterologous | Heterologous | Metabolism | | for the |
| Inventory | Metabolism | Metabolism | Metabolism | Events | | MED |
| casbene | 3 | 0 | 100% | 3.9-79 | ug/gFW | 3 |
| casbene/5- | 4 (3 + 1 of | 5 | 44.4% | 0.10 ug/gFW of | | 16 |
| hydroxy- | only | | | intermediate | | |
| casbene | intermediate | | | product - 1.1 | | |
| | product | | | ug/gFW intended | | |
| | detection) | | | product | | |
| intermedeol | 2 | 5 | 28.6% | 65 ng/gFW- | | 8 |
| | | | | 0.61 ug/gFW | | |
| 11-hydroxy- | 4 | 6 | 40% | 79 ng/gFW- | | 14 |
| vulgarisane + | | | | 328 ug/gFW | | |
| precursors | | | | | | |
| intermedeol + | 1 | 1 | 50% | 2.6 | ug/gFW | 2 |

| | | | | | |
|---|---|---|---|---|---|
| precursors | | | | | |
| 845 (trans- | 0 | 2 | 0% | NA | 2 |
| abienol) | | | | | |
| All MEDs | 14 | 19 | 42.4% | 0.10 ug/gFW of | 45 |
| | | | | intermediate | |
| | | | | product - 328 | |
| | | | | ug/gFW | |

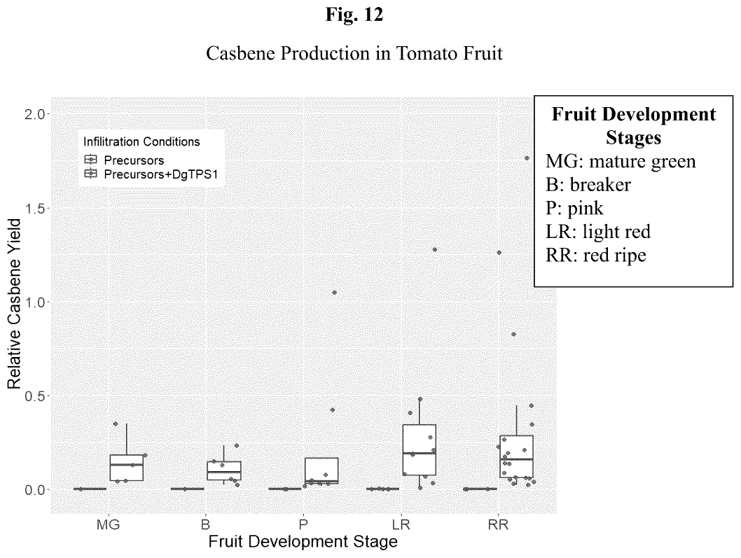

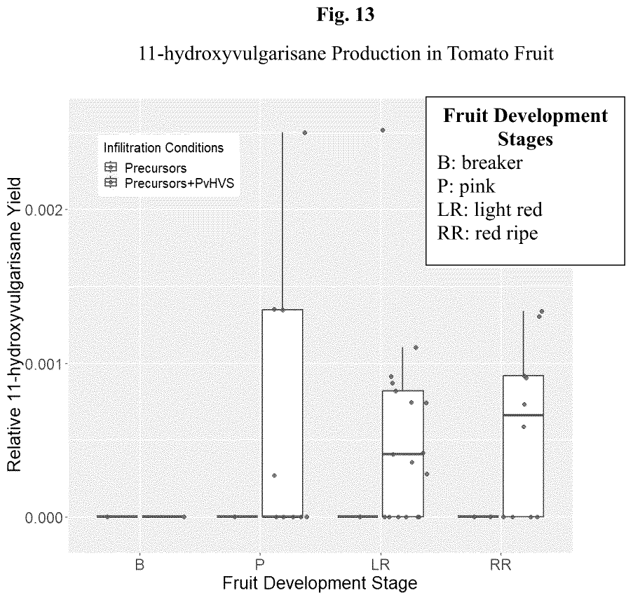

FIG. 12 is a graph showing production of casbene in tomato fruit over different stages of fruit development wherein DbTPS1 (Daphne genkwa terpene synthase 1; SEQ ID NO: 74) is expressed in the tomato fruit tissue. FIG. 13 is a graph showing production of 11-hydroxyvulgarisane in tomato fruit over different stages of fruit development wherein PvHVS (Prunella vulgaris hydroxyvulgarisane synthase; SEQ ID NO: 73) is expressed in the tomato fruit tissue.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

### EMBODIMENTS

Various embodiments of the plants, genomes, methods, biological samples, and other compositions described herein are set forth in the following list of numbered embodiments.
1. A modified tomato plant comprising at least one targeted modification in at least one endogenous terpene or terpenoid biosynthetic gene resulting in decreased expression of the endogenous terpene or terpenoid biosynthetic gene relative to a reference plant lacking the modification,
   wherein the targeted modification in the endogenous terpene or terpenoid biosynthetic gene comprises an insertion, replacement, and/or deletion of one or more nucleotides in the endogenous terpene or terpenoid biosynthetic gene, and
   wherein the decrease of expression in the endogenous terpene or terpenoid biosynthetic gene results in a depletion of carotene compounds in fruit of the tomato relative to the reference tomato plant lacking the modification.
2. The modified tomato plant of embodiment 1, wherein the at least one endogenous terpene or terpenoid biosynthetic gene comprises a beta carotene hydroxylase, a phytoene synthase, a cytochrome P450, a uracil dependent glucosyl transferase, or a combination thereof.
3. The modified tomato plant of embodiments 1 or 2, wherein the endogenous terpene or terpenoid biosynthetic gene comprises a DNA molecule having at least 90%, at least 95%, at least 96%, at least 97%, at least 98%, at least 99%, or 100% sequence identity to the nucleic acid sequence set forth in any one of SEQ ID NOs: 1-44 or an allelic variant thereof.
4. The tomato plant of any one of embodiments 1 to 3, wherein the endogenous terpene or terpenoid biosynthetic gene encodes a polypeptide having at least 90%, at least 95%, at least 96%, at least 97%, at least 98%, at least 99%, or 100% sequence identity to one or more of SEQ ID NOs: 45-66.
5. The tomato plant of any one of embodiments 1 to 4, wherein the tomato plant comprises a loss-of-function allele of the endogenous terpene or terpenoid biosynthetic gene.
6. The tomato plant of any one of embodiments 1 to 5, wherein the one or more targeted modifications are present within the coding region, non-coding region, regulatory sequence, or untranslated region of the endogenous terpene or terpenoid biosynthetic gene.
7. The tomato plant of any one of embodiments 1 to 3, wherein the tomato plant comprises a silencing element that targets the endogenous terpene or terpenoid biosynthetic gene.
8 The tomato plant of any one of embodiments 1 to 7, wherein the tomato plant comprises reduced expression or activity of at least 2, 3, 4, 5, 6, 7, 8, 9, 10, 11, 12, 13, 14, 15, 16, 17, 18, 19, 20, 21, 22, 23, 24, or 25 endogenous terpene or terpenoid biosynthetic genes.
9. The modified tomato plant of any one of embodiments 1 to 8, wherein the endogenous terpene or terpenoid biosynthetic genes comprise:
   (a) beta carotene hydroxylase (CrtR-B2; SEQ ID NO: 2 or 24), phytoene synthase (PSY1; SEQ ID NO: 1 or 23), cytochrome P450_1 (P450_1; SEQ ID NO: 5 or 27), cytochrome P450_2 (P450_2; SEQ ID NO: 6 or 28), cytochrome P450_3 (P450_3; SEQ ID NO: 7 or 29), cytochrome P450_4 (P450_4; SEQ ID NO: 8 or 30), and uracil dependent glucosyl transferase-4 (UGT_4; SEQ ID NO: 17 or 39); or
   (b) uracil dependent glucosyl transferase-6 (UGT_6; SEQ ID NO: 19 or 41), uracil dependent glucosyl transferase-7 (UGT_7; SEQ ID NO: 20 or 42), uracil dependent glucosyl transferase-8 (UGT_8; SEQ ID NO: 21 or 43), uracil dependent glucosyl transferase-9 (UGT_9; SEQ ID NO: 22 or 44), cytochrome P450_8 (P450_8; SEQ ID NO: 12 or 34), and cytochrome P450_9 (P450_9; SEQ ID NO: 13 or 35).
10. The modified tomato plant of any one of embodiments 1 to 9, wherein the depleted carotene comprises triterpenes and tetraterpenes.
11. The modified tomato plant of any one of embodiments 1 to 10, wherein the at least one targeted modification in the at least one endogenous terpene or terpenoid biosynthetic gene is non-naturally occurring.
12. The modified tomato plant of any one of embodiments 1 to 11, wherein the plant is not exclusively obtained by means of an essentially biological process.
13. The modified tomato plant of any one of embodiments 1 to 12, wherein the modified tomato plant further comprises a heterologous terpene or terpenoid biosynthetic gene.
14. The modified tomato plant of embodiment 13, wherein the heterologous terpene or terpenoid biosynthetic gene is expressed in the tomato fruit.
15. The modified tomato plant of embodiments 13 or 14, wherein the heterologous terpene or terpenoid biosynthetic gene comprises a heterologous terpene synthase, cytochrome P450, uracil dependent glucosyl transferase, or a combination thereof.
16. The modified tomato plant of any one of embodiments 13 to 15, wherein the heterologous terpene or terpenoid biosynthetic gene comprises a nucleotide sequence having at least 90%, at least 95%, at least 96%, at least 97%, at least 98%, at least 99%, or 100% sequence identity to one or more of SEQ ID NOs: 67-75.
17. The tomato plant of any one of embodiments 13 to 16, wherein the heterologous terpene or terpenoid biosynthetic gene encodes a polypeptide having at least 90%, at least 95%, at least 96%, at least 97%, at least 98%, at least 99%, or 100% sequence identity to one or more of SEQ ID NOs: 76-84.
18. The modified tomato plant of embodiment 17, wherein the heterologous terpene or terpenoid biosynthetic gene comprises CfTPS2 (SEQ ID NO: 67), OmTPS3 (SEQ ID NO: 68), CfDXS (SEQ ID NO: 69), CfGPPS (SEQ ID NO: 70), TP-AIFPPS (SEQ ID NO: 71), TP-SbTPS10 (SEQ ID NO: 72), PvHVS (SEQ ID NO: 73), DgTPS1 (SEQ ID NO: 74), ElCYP726A27 (SEQ ID NO: 75), and combinations thereof.
19. The tomato plant of any one of embodiments 13 to 18, wherein the heterologous terpene or terpenoid biosynthetic gene is operably linked to a promoter functional in a tomato plant cell.
20. The tomato plant of embodiment 19, wherein the promoter is a tomato fruit-specific promoter.
21. A plant part, a plant cell, a seed, an asexual propagate, or a progeny of the tomato plant of any one of embodiments 1-20.
22. The plant part of embodiment 21, wherein the plant part is a tomato fruit.
23. A modified tomato plant cell containing a chromosome comprising the targeted modification(s) in the at least one terpenoid biosynthetic gene of any one of embodiments 1 to 12.
24. A tissue culture of regenerable cells comprising the modified tomato plant cell of embodiment 23.
25. A method of producing a modified tomato plant material, comprising:
(a) introducing a targeted modification into at least one endogenous terpene or terpenoid biosynthetic gene into a tomato plant to produce a modified tomato plant with decreased expression of the endogenous terpenoid biosynthetic gene relative to a reference tomato plant lacking the modification,
   wherein the targeted modification in the endogenous terpenoid biosynthetic gene comprises an insertion, replacement, and/or deletion of one or more nucleotides in the endogenous terpenoid biosynthetic gene, and
   wherein the decrease of expression in the endogenous terpenoid biosynthetic gene results in a depletion of carotene in fruit of the tomato plant relative to the reference tomato plant lacking the modification; and
(b) growing the modified tomato plant under conditions that allow for decreased or absent expression of the at least one target endogenous terpene or terpenoid biosynthetic gene relative to the reference tomato plant which lacks the modifications.
26 The method of embodiment 25, wherein the method comprises introducing the targeted modification at a genomic locus comprising the at least one endogenous terpene or terpenoid biosynthetic gene.
27. The method of embodiments 24 or 25, wherein the targeted modifications are present within the coding region, non-coding region, regulatory sequence, or untranslated region of the endogenous terpene or terpenoid biosynthetic gene.
28. The method of any one of embodiments 25 to 27, wherein the targeted modifications are introduced through targeted DNA modification through use of an RNA-guided endonuclease and a guide RNA.
29. The method of any one of embodiments 25 to 28, wherein the at least one endogenous terpene or terpenoid biosynthetic gene comprises a beta carotene hydroxylase, a phytoene synthase, a cytochrome P450, a uracil dependent glucosyl transferase, or a combination thereof.
30. The method of any one of embodiments 25 to 29, wherein the endogenous terpene or terpenoid biosynthetic gene comprises a DNA molecule having at least 90%, at least 95%, at least 96%, at least 97%, at least 98%, at least 99%, or 100% sequence identity to the nucleic acid sequence set forth in any one of SEQ ID NOs: 1-44 or an allelic variant thereof.
31. The method of any one of embodiments 25 to 30, wherein the endogenous terpene or terpenoid biosynthetic genes comprise:
   (a) beta carotene hydroxylase (CrtR-B2; SEQ ID NO: 2 or 24), phytoene synthase (PSY1; SEQ ID NO: 1 or 23), cytochrome P450_1 (P450_1; SEQ ID NO: 5 or 27), cytochrome P450_2 (P450_2; SEQ ID NO: 6 or 28), cytochrome P450_3 (P450_3; SEQ ID NO: 7 or 29), cytochrome P450_4 (P450_4; SEQ ID NO: 8 or 30), and uracil dependent glucosyl transferase-4 (UGT_4; SEQ ID NO: 17 or 39); or
   (b) uracil dependent glucosyl transferase-6 (UGT_6; SEQ ID NO: 19 or 41), uracil dependent glucosyl transferase-7 (UGT_7; SEQ ID NO: 20 or 42), uracil dependent glucosyl transferase-8 (UGT_8; SEQ ID NO: 21 or 43), uracil dependent glucosyl transferase-9 (UGT_9; SEQ ID NO: 22 or 44), cytochrome P450_8 (P450_8; SEQ ID NO: 12 or 34), and cytochrome P450_9 (P450_9; SEQ ID NO: 13 or 35).
32. The method of any one of embodiments 25 to 31, wherein the modified tomato plant further comprises a heterologous terpene or terpenoid biosynthetic gene.
33. The method of embodiment 32, wherein the heterologous terpene or terpenoid biosynthetic gene is operably linked to a tissue specific promoter functional in a tomato plant fruit cell.
34. The method of embodiments 32 or 33, wherein the heterologous terpene or terpenoid biosynthetic gene comprises a heterologous terpene synthase, cytochrome P450, uracil dependent glucosyl transferase, or a combination thereof.
35. The method of any one of embodiments 32 to 34, wherein the heterologous terpene or terpenoid biosynthetic gene comprises a nucleotide sequence having at least 90%, at least 95%, at least 96%, at least 97%, at least 98%, at least 99%, or 100% sequence identity to one or more of SEQ ID NOs: 67-75.
36. The method of any one of embodiments 32 to 35, wherein the heterologous terpene or terpenoid biosynthetic gene comprises CfTPS2 (SEQ ID NO: 67), OmTPS3 (SEQ ID NO: 68), CfDXS (SEQ ID NO: 69), CfGPPS (SEQ ID NO: 70), TP-AIFPPS (SEQ ID NO: 71), TP-SbTPS10 (SEQ ID NO: 72), PvHVS (SEQ ID NO: 73), DgTPS1 (SEQ ID NO: 74), ElCYP726A27 (SEQ ID NO: 75), and combinations thereof.
37. The method of any one of embodiments 32 to 36, further comprising isolating a terpene or terpenoid from the fruit of the modified tomato.

### REFERENCES

References in the present disclosure are as follows:
1. Lee, D. W., Lee, S., Lee, G. J., Lee, K. H., Kim, S., Cheong, G. W., & Hwang, I. (2006). Functional characterization of sequence motifs in the transit peptide of Arabidopsis small subunit of rubisco. Plant physiology, 140 (2), 466-483. doi.org/10.1104/pp.105.074575
2. Johnson, S. R., Bhat, W. W., Bibik, J., Turmo, A., Hamberger, B., Evolutionary Mint Genomics Consortium, & Hamberger, B. (2019). A database-driven approach identifies additional diterpene synthase activities in the mint family (Lamiaceae). The Journal of biological chemistry, 294 (4), 1349-1362. doi.org/10.1074/jbc.RA118.006025
3. Sun, H., Lang, Z., Zhu, L., & Huang, D. (2012). Acquiring transgenic tobacco plants with insect resistance and glyphosate tolerance by fusion gene transformation. Plant cell reports, 31 (10), 1877-1887. doi.org/10.1007/s00299-012-1301-5
4. Bibik, J. D., Weraduwage, S. M., Banerjee, A., Robertson, K., Espinoza-Corral, R., Sharkey, T. D., Lundquist, P. K., & Hamberger, B. R. (2022). Pathway Engineering, Re-targeting, and Synthetic Scaffolding Improve the Production of Squalene in Plants. ACS synthetic biology, 11(6), 2121-2133. doi.org/10.1021/acssynbio.2c00051
5. Luo, D., Callari, R., Hamberger, B., Wubshet, S. G., Nielsen, M. T., Andersen-Ranberg, J., Hallström, B. M., Cozzi, F., Heider, H., Lindberg Møller, B., Staerk, D., & Hamberger, B. (2016). Oxidation and cyclization of casbene in the biosynthesis of Euphorbia factors from mature seeds of Euphorbia lathyris L. Proceedings of the National Academy of Sciences of the United States of America, 113 (34), E5082-E5089. doi.org/10.1073/pnas. 1607504113
6. Johnson, S. R., Bhat, W. W., Sadre, R., Miller, G. P., Garcia, A. S., & Hamberger, B. (2019). Promiscuous terpene synthases from Prunella vulgaris highlight the importance of substrate and compartment switching in terpene synthase evolution. The New phytologist, 223 (1), 323-335. doi.org/10.1111/nph. 15778
7. Andersen-Ranberg, J., Kongstad, K. T., Nielsen, M. T., Jensen, N. B., Pateraki, I., Bach, S. S., Hamberger, B., Zerbe, P., Staerk, D., Bohlmann, J., Møller, B. L., & Hamberger, B. (2016). Expanding the Landscape of Diterpene Structural Diversity through Stereochemically Controlled Combinatorial Biosynthesis. Angewandte Chemie (International ed. in English), 55 (6), 2142-2146. doi.org/10.1002/anie.201510650
8. Keim, V., Manzano, D., Fernández, F. J., Closa, M., Andrade, P., Caudepón, D., Bortolotti, C., Vega, M. C., Arró, M., & Ferrer, A. (2012). Characterization of Arabidopsis FPS isozymes and FPS gene expression analysis provide insight into the biosynthesis of isoprenoid precursors in seeds. PloS one, 7 (11), e49109. doi.org/10.1371/journal.pone.0049109
9. Bekele, D., Tesfaye, K., and Fikre, A. (2019). Applications of Virus Induced Gene Silencing (VIGS) in Plant Functional Genomics Studies. J Plant Biochem Physiol, 7 (1), doi: 10.4172/2329-9029.1000229
10. Chatzivasileiou, A. O., V. Ward, S. M. Edgar and G. Stephanopoulos (2019). “Two-step pathway for isoprenoid synthesis.” Proc Natl Acad Sci USA 116 (2): 506-511.
11. Orzaez, D., Mirabel, S., Wieland, W. H., Granell, A. (2006). Agroinjection of Tomato Fruits. A Tool for Rapid Functional Analysis of Transgenes Directly in Fruit. Plant Physiology, 140 (1): 3-11.
12. Atkinson, R. G., Bolitho, K. M., Wright, M. A. et al. Apple ACC-oxidase and polygalacturonase: ripening-specific gene expression and promoter analysis in transgenic tomato. Plant Mol Biol 38, 449-460 (1998). doi.org/10.1023/A: 100606592639713.
13. Xue-Ming Ruan, Xiangyu Xiong, Jian-Feng Li, Identification and application of an exocarp-preferential promoter for genetic engineering of tomato fruit, Horticulture Research, Volume 11, Issue 3, March 2024, uhae035, doi.org/10.1093/hr/uhae035.

All cited patents and patent publications referred to in this application are incorporated herein by reference in their entirety. All of the materials and methods disclosed and claimed herein can be made and used without undue experimentation as instructed by the above disclosure and illustrated by the examples. Although the materials and methods of this disclosure have been described in terms of embodiments and illustrative examples, it will be apparent to those of skill in the art that substitutions and variations can be applied to the materials and methods described herein without departing from the concept, spirit, and scope of the disclosure. All such similar substitutes and modifications apparent to those skilled in the art are deemed to be within the spirit, scope, and concept of the disclosure as encompassed by the embodiments of the disclosures recited herein and the specification and appended claims.

What is claimed is:

1. A modified tomato plant comprising at least one targeted modification in at least one endogenous terpene or terpenoid biosynthetic gene resulting in decreased expression of the endogenous terpene or terpenoid biosynthetic gene relative to a reference plant lacking the modification,
wherein the targeted modification in the endogenous terpene or terpenoid biosynthetic gene comprises an insertion, replacement, and/or deletion of one or more nucleotides in the endogenous terpene or terpenoid biosynthetic gene, and
wherein the decrease of expression in the endogenous terpene or terpenoid biosynthetic gene results in a depletion of carotene compounds in fruit of the tomato relative to the reference tomato plant lacking the modification.

2. The modified tomato plant of claim 1, wherein the at least one endogenous terpene or terpenoid biosynthetic gene comprises a beta carotene hydroxylase, a phytoene synthase, a cytochrome P450, a uracil dependent glucosyl transferase, or a combination thereof.

3. The modified tomato plant of claim 1, wherein the endogenous terpene or terpenoid biosynthetic gene comprises a DNA molecule having at least 90%, at least 95%, at least 96%, at least 97%, at least 98%, at least 99%, or 100% sequence identity to the nucleic acid sequence set forth in any one of SEQ ID NOs: 1-44 or an allelic variant thereof.

4. The modified tomato plant of claim 1, wherein the endogenous terpene or terpenoid biosynthetic genes comprise:
(a) beta carotene hydroxylase (CrtR-B2; SEQ ID NO: 2 or 24), phytoene synthase (PSY1; SEQ ID NO: 1 or 23), cytochrome P450_1 (P450_1; SEQ ID NO: 5 or 27), cytochrome P450_2 (P450_2; SEQ ID NO: 6 or 28), cytochrome P450_3 (P450_3; SEQ ID NO: 7 or 29), cytochrome P450_4 (P450_4; SEQ ID NO: 8 or 30), and uracil dependent glucosyl transferase-4 (UGT_4; SEQ ID NO: 17 or 39); or
(b) uracil dependent glucosyl transferase-6 (UGT_6; SEQ ID NO: 19 or 41), uracil dependent glucosyl transferase-7 (UGT_7; SEQ ID NO: 20 or 42), uracil dependent glucosyl transferase-8 (UGT_8; SEQ ID NO: 21 or 43), uracil dependent glucosyl transferase-9 (UGT_9; SEQ ID NO: 22 or 44), cytochrome P450_8 (P450_8; SEQ ID NO: 12 or 34), and cytochrome P450_9 (P450_9; SEQ ID NO: 13 or 35).

5. The modified tomato plant of claim 1, wherein the at least one targeted modification in the at least one endogenous terpene or terpenoid biosynthetic gene is non-naturally occurring.

6. The modified tomato plant of claim 1, wherein the plant is not exclusively obtained by means of an essentially biological process.

7. The modified tomato plant of claim 1, wherein the modified tomato plant further comprises a heterologous terpene or terpenoid biosynthetic gene.

8. The modified tomato plant of claim 7, wherein the heterologous terpene or terpenoid biosynthetic gene is expressed in the tomato fruit.

9. The modified tomato plant of claim 7, wherein the heterologous terpene or terpenoid biosynthetic gene comprises a heterologous terpene synthase, cytochrome P450, uracil dependent glucosyl transferase, or a combination thereof.

10. The modified tomato plant of claim 7, wherein the heterologous terpene or terpenoid biosynthetic gene comprises a nucleotide sequence having at least 90%, at least 95%, at least 96%, at least 97%, at least 98%, at least 99%, or 100% sequence identity to one or more of SEQ ID NOs: 67-75.

11. A modified tomato plant cell containing a chromosome comprising the targeted modification(s) in the at least one terpene or terpenoid biosynthetic gene of claim 3.

12. A tissue culture of regenerable cells comprising the modified tomato plant cell of claim 11.

11. A method of producing a modified tomato plant material, comprising:
(a) introducing a targeted modification into at least one endogenous terpene or terpenoid biosynthetic gene into a tomato plant to produce a modified tomato plant with decreased expression of the endogenous terpenoid biosynthetic gene relative to a reference tomato plant lacking the modification,
wherein the targeted modification in the endogenous terpenoid biosynthetic gene comprises an insertion, replacement, and/or deletion of one or more nucleotides in the endogenous terpenoid biosynthetic gene, and
wherein the decrease of expression in the endogenous terpenoid biosynthetic gene results in a depletion of carotene in fruit of the tomato plant relative to the reference tomato plant lacking the modification; and
(b) growing the modified tomato plant under conditions that allow for decreased or absent expression of the at least one target endogenous terpene or terpenoid biosynthetic gene relative to the reference tomato plant which lacks the modifications.

12. The method of claim 11, wherein the method comprises introducing the targeted modification at a genomic locus comprising the at least one endogenous terpene or terpenoid biosynthetic gene.

13. The method of claim 12, wherein the targeted modifications are present within the coding region, non-coding region, regulatory sequence, or untranslated region of the endogenous terpene or terpenoid biosynthetic gene.

14. The method of claim 12, wherein the targeted modifications are introduced through targeted DNA modification through use of an RNA-guided endonuclease and a guide RNA.

15. The method of claim 11, wherein the at least one endogenous terpene or terpenoid biosynthetic gene comprises a beta carotene hydroxylase, a phytoene synthase, a cytochrome P450, a uracil dependent glucosyl transferase, or a combination thereof.

16. The method of claim 11, wherein the endogenous terpene or terpenoid biosynthetic gene comprises a DNA molecule having at least 90%, at least 95%, at least 96%, at least 97%, at least 98%, at least 99%, or 100% sequence identity to the nucleic acid sequence set forth in any one of SEQ ID NOs: 1-44 or an allelic variant thereof.

17. The method of claim 11, wherein the endogenous terpene or terpenoid biosynthetic genes comprise:
(a) beta carotene hydroxylase (CrtR-B2; SEQ ID NO: 2 or 24), phytoene synthase (PSY1; SEQ ID NO: 1 or 23), cytochrome P450_1 (P450_1; SEQ ID NO: 5 or 27), cytochrome P450_2 (P450_2; SEQ ID NO: 6 or 28), cytochrome P450_3 (P450_3; SEQ ID NO: 7 or 29), cytochrome P450_4 (P450_4; SEQ ID NO: 8 or 30), and uracil dependent glucosyl transferase-4 (UGT_4; SEQ ID NO: 17 or 39); or
(b) uracil dependent glucosyl transferase-6 (UGT_6; SEQ ID NO: 19 or 41), uracil dependent glucosyl transferase-7 (UGT_7; SEQ ID NO: 20 or 42), uracil dependent glucosyl transferase-8 (UGT_8; SEQ ID NO: 21 or 43), uracil dependent glucosyl transferase-9 (UGT_9; SEQ ID NO: 22 or 44), cytochrome P450_8 (P450_8; SEQ ID NO: 12 or 34), and cytochrome P450_9 (P450_9; SEQ ID NO: 13 or 35).

18. The method of claim 11, wherein the modified tomato plant further comprises a heterologous terpene or terpenoid biosynthetic gene.

19. The method of claim 18, wherein the heterologous terpene or terpenoid biosynthetic gene is operably linked to a tissue specific promoter functional in a tomato plant fruit cell.

20. The method of claim 18, wherein the heterologous terpene or terpenoid biosynthetic gene comprises a heterologous terpene synthase, cytochrome P450, uracil dependent glucosyl transferase, or a combination thereof.

21. The method of claim 18, wherein the heterologous terpene or terpenoid biosynthetic gene comprises a nucleotide sequence having at least 90%, at least 95%, at least 96%, at least 97%, at least 98%, at least 99%, or 100% sequence identity to one or more of SEQ ID NOs: 67-75.

22. The method of claim 1, further comprising isolating a terpene or terpenoid from the fruit of the modified tomato.
